SEQUENCE LISTING (255 sequences)

SEQ ID NO: 1 - protein, 112 aa, unidentified
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 2 - protein, 98 aa, synthetic construct
APTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVP

SEQ ID NO: 3 - protein, 15 aa, synthetic construct
EPKSCDKTHTCPPCP

SEQ ID NO: 4 - protein, 12 aa, synthetic construct
ERKCCVECPPCP

SEQ ID NO: 5 - protein, 12 aa, synthetic construct
ESKYGPPCPSCP

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 15 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 21 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 23 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 27 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 235 aa, synthetic construct; identical to SEQ ID NO: 15

SEQ ID NO: 29 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 32 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 33 - protein, 329 aa, synthetic construct
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 328 aa, synthetic construct
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 332 aa, synthetic construct
PLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 331 aa, synthetic construct
PLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 330 aa, synthetic construct
PLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 329 aa, synthetic construct
PLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 15 aa, synthetic construct
GGGGSGGGGSGGGGS

SEQ ID NO: 40 - protein, 119 aa, synthetic construct
QVQLVESGGGVVQPGKSLRLSCVASGFTFSSDAMYWVRQAPGKGLEWVAVIYYDGNYQYYEDSVKGRFTISRDNSQNTLDLQMNSLRVDDTAVYFCARLNWDYWYLDLWGRGTLVTVSS

SEQ ID NO: 41 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 42 - protein, 119 aa, synthetic construct
QVQLVESGGGVVQPGRSLRLSCTASGFTFSSYAMYWVRQAPGKGLEWVSVIYYDGSYKYYADSVKGRFTISRDNSKNTLYLQMDSLRAEDTAVYYCASYNWNYWYFDFWGRGTLVTVSS

SEQ ID NO: 43 - protein, 119 aa, synthetic construct
QVQLVESGGSVVQPGRSLRLSCAASGFTFSTYAMYWVRQTPGKGLEWVAVLYSDGSNKYYIDSVKGRFTISRDTSTNTLYLQMSSLRADDSALYYCARLNWDYWYFDLWGRGTLVTVSS

SEQ ID NO: 44 - protein, 119 aa, synthetic construct
QVQLVESGGGVVQPGRSLRLSCEASGFSSSDNAMYWVRQAPGKGLEWVSVIYHDGSYKYYEDSVKGRFTIARDNSKNTLYLQMNSLRAEDTAVYYCARYNWNHWYFDVWGRGTLVTVSS

SEQ ID NO: 45 - protein, 122 aa, synthetic construct
QVQLVESGGGVVQPGRSLRLSCAASGFTFSTYGMHWVRQAPGKGLEWVSVISYDESNKYYADSVKGRFTISRDNSKNALYLQMNSLRNEDTAVYYCARDRPFGLVTGWFDPWGQGTLVTVSS

SEQ ID NO: 46 - protein, 120 aa, synthetic construct
QVQLVESGGGVVQPGRSLRLSCAASGFSFNTYGMHWVRQAPGKGLEWVTIIWYDGSIKYYADSVKGRFTISRDNSKNTLYLQMNSLRAEDTAVYYCARGGYSGYLYFDYWGQGTLVTVSS

SEQ ID NO: 47 - protein, 124 aa, synthetic construct
QVQLQESGPGLVKPSQTLSLTCTVSGGSISSGGDYWSWIRQLPGKGLEWIGYIYYSGSAYYNPSLKSRGTISIDTSKNQFSLKLTSVTAADTAVYFCVKLRFLEWFLGGWFGPWGQGTLVTVSS

SEQ ID NO: 48 - protein, 120 aa, synthetic construct
EVQLVESGGGLVQPGGSLRLSCVASGFTFNKYDMHWVRQTTGKGLEWVSGIDTDGDTYYPGSVKGRFTISRENAENSLYLQMNGLRVGDTAVYYCARWPWSGFYGAFDIWGQGTMVTVSS

SEQ ID NO: 49 - protein, 122 aa, synthetic construct
QVQLQESGPGLVKPSQTLSLTCTVSGGSISSGNYYWNWIRQQPGEGLEWIAYIYHNGVTNFNPSLKSRLTISVDTSKTQFSLKLRSVTAADTAVYYCARSGSWFENWYFDLWGRGTLVTVSS

SEQ ID NO: 50 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 51 - protein, 120 aa, synthetic construct
QVQLQESGPGLVKPSETLSLTCTVSGGSISNSYWSWIRQPPGKGLEWIGYVYSRGNTKYNPSLTSRVTMSFDTSKNQFSLKLRSVTAADTAVYYCARSSSWYEDWYFDLWGRGTLVTVSS

SEQ ID NO: 52 - protein, 118 aa, synthetic construct
QVQLQQWGAGLFKPSETLSLTCDVYGGSFRGYYWSWIRQPPGKGLEWIGEISYSGFTNYNPSLKSRVIISIDTSKNQFSLKMSSVTAADTAVYYCARVTYGYGTFDYWGQGTLVTVSS

SEQ ID NO: 53 - protein, 116 aa, synthetic construct
EVQLVQSGAEVKKPGSSVKVSCKASGYTFTDYYINWVRQAPGQGLEWIGWIFPGSGSVYCNEQFKGRATLTVDRSTSTAYMELSSLRSEDTAVYFCASSLGKFAYWGQGTLVTVSS

SEQ ID NO: 54 - protein, 107 aa, synthetic construct
DIQMTQSPSSLSASVGDRVTITCKASQSVVTAVAWYQQKPGKSPKLLIYSASNRYTGVPSRFSGSGSGTDFTLTISSLQPEDFATYFCQQYSSYPYTFGGGTKVEIK

SEQ ID NO: 55 - protein, 116 aa, synthetic construct
EVQLVQSGAEVKKPGSSVKVSCKASGYTFTDYYINWVRQAPGQGLEWIGWIFPGSGSVYSNEQFKGRATLTVDRSTSTAYMELSSLRSEDTAVYFCASSLGKFAYWGQGTLVTVSS

SEQ ID NO: 56 - protein, 116 aa, synthetic construct
EVQLVQSGAEVKKPGSSVKVSCKASGYTFTDYYINWVRQAPGQGLEWIGWIFPGSGSVYCNEQFKGRVTITVDKSTSTAYMELSSLRSEDTAVYYCASSLGKFAYWGQGTLVTVSS

SEQ ID NO: 57 - protein, 112 aa, synthetic construct
EVQLQESGPGLVKPSETLSLTCTVSGYSITSNYYWNWIRQPPGKGLEWMGYIRYDGSNNYNPSLKNRITISRDTSKNQFSLKLSSVTAADTAVYYCARLDYWGQGTTVTVSS

SEQ ID NO: 58 - protein, 112 aa, synthetic construct
DAVMTQTPLSLSVTPGQPASISCRSSQSLENTNGNTFLNWFLQKPGQSPQLLIYRVSNRFSGVPDRFSGSGSGTDFTLKISRVEAEDVGVYFCLQVTHVPFTFGQGTKLEIK

SEQ ID NO: 59 - protein, 112 aa, synthetic construct
EVQLQESGPGLVKPSETLSLTCTVSGYSITSNYYWNWIRQPPGKGLEWMGYIRYDGSNNYNPSLKNRVTISRDTSKNQFSLKLSSVTAADTAVYYCARLDYWGQGTTVTVSS

SEQ ID NO: 60 - protein, 112 aa, synthetic construct
EVQLQESGPGLVKPSETLSLTCTVSGYSISSNYYWNWIRQPPGKGLEWMGYIRYDGSNNYNPSLKSRVTISRDTSKNQFSLKLSSVTAADTAVYYCARLDYWGQGTTVTVSS

SEQ ID NO: 61 - protein, 112 aa, synthetic construct
DAVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 62 - protein, 112 aa, synthetic construct
EVQLVESGGGLVKPGETLSLTCTVSGYSITSNYYWNWIRQPPGKGLEWMGYIRYDGSNNYNPSLKGRVTISRDTSKNQFYLKLSSVTAADTAVYYCARLDYWGQGTTVTVSS

SEQ ID NO: 63 - protein, 112 aa, synthetic construct
DAVMTQTPLSLSVTEGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 64 - protein, 112 aa, synthetic construct
EVQLVESGGGLVQPGGSLRLSCAASGYSITSNYYWNWIRQPPGKGLEWMGYIRYDGSNNYNPSLKGRVTISRDTSKNQLYLKLSSVTAADTAVYYCARLDYWGQGTLVTVSS

SEQ ID NO: 65 - protein, 112 aa, synthetic construct
EVQLVESGGGLVKPGETLILTCTVSGYDITSNYYWNWIRQPPGKGLEWMGYIRYDGSNNYNPSLKGRVTISRDTSKNQFYLKLSSVTAADTAVYYCARLDYWGQGTTVTVSS

SEQ ID NO: 66 - protein, 112 aa, synthetic construct
DAVMTQTPLSLAVLPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 67 - protein, 112 aa, synthetic construct
QVQLQESGPGLVKPSETLSLTCTVSGYSITSNYYWNWIRQPPGKGLEWMGYIRYDGSNNYNPSLKNRITISRDTSKNQFSLKLSSVTAADTAVYYCARLDYWGQGTLVTVSS

SEQ ID NO: 68 - protein, 112 aa, synthetic construct
DIQMTQSPSSLSASVGDRVTITCRSSQSLENTNGNTFLNWYQQKPGKAPKLLIYRVSNRFSGVPSRFSGSGSGTDFTLTISSLQPEDFATYYCLQVTHVPFTFGQGTKVEIK

SEQ ID NO: 69 - protein, 112 aa, synthetic construct
DAVMTQSPLSLPVTLGEPASISCRSSQSLENTNGNTFLNWFQQKPGQSPRLLIYRVSNRFSGVPDRFSGSGSGTDFTLKISRVEAEDVGVYYCLQVTHVPFTFGQGTKLEIK

SEQ ID NO: 70 - protein, 112 aa, synthetic construct
EVQLVESGGGLVQPGGSLRLSCAASGYSITSNYYWNWVRQAPGKGLEWMGYIRYDGSNNYNPSLKNRITISRDTSKNTFYLQMNSLRAEDTAVYYCARLDYWGQGTLVTVSS

SEQ ID NO: 71 - protein, 112 aa, synthetic construct
DAQMTQSPSSLSASVGDRVTITCRSSQSLENTNGNTFLNWFQQKPGKAPKLLIYRVSNRFSGVPSRFSGSGSGTDFTLTISSLQPEDFATYYCLQVTHVPFTFGQGTKLEIK

SEQ ID NO: 72 - protein, 112 aa, synthetic construct
DIVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 73 - protein, 112 aa, synthetic construct
DVVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 74 - protein, 117 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 75 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 76 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQAHTLPPTFGQGTKVEIK

SEQ ID NO: 77 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLAPTFGQGTKVEIK

SEQ ID NO: 78 - protein, 117 aa, synthetic construct
EVQLQQSGPELVKPGASVKISCKASGYTFTDSYMSWVKQSHGKTLEWIGDMYPDNGDSSYNQKFREKVTLTVDKSSTTAYMEFRSLTSEDSAVYYCVLAPRWYFSVWGTGTTVTVSS

SEQ ID NO: 79 - protein, 107 aa, synthetic construct
DIQMTQTTSSLSASLGDRVTISCRASQDISNYLNWYQQKPDGTVKLLIYYTSRLRSGVPSRFSGSGSGKDYFLTISNLEQEDVAAYFCQQGHTLPPTFGGGTKLEIK

SEQ ID NO: 80 - protein, 114 aa, synthetic construct
QVQLQQSGAELVRPGTSVKVSCKASGYAFTNYLIEWVKQRPGQGLEWIGVINPGSGDTYYSEKFKGKVTLTADKSSSTAYMQLSSLTSEDSAVYFCARDRLDYWGQGTTLTVSS

SEQ ID NO: 81 - protein, 107 aa, synthetic construct
DILMTQSPSSMSVSLGDTVSITCHASQDISSYIVWLQQKPGKSFRGLIYHGTNLEDGIPSRFSGSGSGADYSLTISSLESEDFADYYCVHYAQFPYTFGGGTKLEIK

SEQ ID NO: 82 - protein, 118 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 83 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 84 - protein, 124 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSS

SEQ ID NO: 85 - protein, 106 aa, synthetic construct
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 86 - protein, 122 aa, synthetic construct
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 87 - protein, 107 aa, synthetic construct
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 88 - protein, 120 aa, synthetic construct
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 89 - protein, 111 aa, synthetic construct
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 90 - protein, 138 aa, synthetic construct
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 91 - protein, 126 aa, synthetic construct
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 92 - protein, 119 aa, synthetic construct
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCANYYGSSLSMDYWGQGTSVTVSS

SEQ ID NO: 93 - protein, 108 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 94 - protein, 121 aa, synthetic construct
EVQLQQSGPELVKPGASVKISCKTSGYTFKDYTMHWVKQSHGKSLEWIGGIYPNNGGSTYNQNFKDKATLTVDKSSSTAYMEFRSLTSEDSAVYYCARMGYHGPHLDFDVWGAGTTVTVSP

SEQ ID NO: 95 - protein, 108 aa, synthetic construct
DIVMTQSHKFMSTSLGDRVSITCKASQDVGAAVAWYQQKPGQSPKLLIYWASTRHTGVPDRFTGGGSGTDFTLTISNVQSEDLTDYFCQQYINYPLTFGGGTKLEIKR

SEQ ID NO: 96 - protein, 119 aa, synthetic construct
QVQLVQSGAEVKKPGASVKVSCKASGYTFTSYVMHWVRQAPGQRLEWMGYINPYNDGTKYNEKFKGRVTITSDTSASTAYMELSSLRSEDTAVYYCANYYGSSLSMDYWGQGTLVTVSS

SEQ ID NO: 97 - protein, 108 aa, synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPWTFGQGTKVEIKR

SEQ ID NO: 98 - protein, 119 aa, synthetic construct
QVQLVQSGAEVKKPGASVKVSCKASGYTFTSYVMHWVRQAPGQRLEWIGYINPYNDGTKYNEKFKGRATITSDTSASTAYMELSSLRSEDTAVYYCANYYGSSLSMDYWGQGTLVTVSS

SEQ ID NO: 99 - protein, 108 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPWTFGQGTKVEIKR

SEQ ID NO: 100 - protein, 119 aa, synthetic construct
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSS

SEQ ID NO: 101 - protein, 121 aa, synthetic construct
QVQLVQSGAEVKKPGSSVKVSCKASGYTFKDYTMHWVRQAPGQGLEWMGGIYPNNGGSTYNQNFKDRVTITADKSTSTAYMELSSLRSEDTAVYYCARMGYHGPHLDFDVWGQGTTVTVSS

SEQ ID NO: 102 - protein, 108 aa, synthetic construct
DIQMTQSPSSLSASVGDRVTITCKASQDVGAAVAWYQQKPGKAPKLLIYWASTRHTGVPSRFSGSGSGTDFTLTISSLQPEDFATYYCQQYINYPLTFGGGTKVEIKR

SEQ ID NO: 103 - protein, 108 aa, synthetic construct
DIQMTQSPSSLSASVGDRVTITCKASQDVGAAVAWYQQKPGKAPKLLIYWASTRHTGVPDRFSGGGSGTDFTLTISSLQPEDFATYYCQQYINYPLTFGGGTKVEIKR

SEQ ID NO: 104 - protein, 121 aa, synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARMGYHGPHLDFDVWGQGTTVTVSS

SEQ ID NO: 105 - protein, 121 aa, synthetic construct
QVQLVQSGAEVKKPGSSVKVSCKASGYTFKDYTMHWVRQAPGQGLEWIGGIYPNNGGSTYNQNFKDRATLTVDKSTSTAYMELSSLRSEDTAVYYCARMGYHGPHLDFDVWGQGTTVTVSS

SEQ ID NO: 106 - protein, 128 aa, synthetic construct
QVQLVQSGAEVKKPGASVKVSCKASGYTFTNYHLHWVRQAPGQGLEWMGMINPNDGSTTYAQKFQGRVTMTRDTSTSTVYMELSSLRSEDTAVYYCARSTYYYDSSGYYYYYYGMDVWGQGTTVTVSS

SEQ ID NO: 107 - protein, 108 aa, synthetic construct
EIVMTQSPATLSVSPGERATLSCRASQSVYSNYLAWYQQKPGQAPRLLIYGASTRATGIPARFSGSGSGTEFTLTISSLQSEDFAVYYCQQYHSYPLTFGGGTKVEIK

SEQ ID NO: 108 - protein, 115 aa, synthetic construct
QVQLVQSGAEVKKPGSSVKVSCKASGYRFTGYHLHWVRQAPGQGLEWMGGIIPIFGTANYAQKFQGRVTITADKSTSTAYMELSSLRSEDTAVYYCAYGVPPDPWGQGTLVTVSS

SEQ ID NO: 109 - protein, 112 aa, synthetic construct
DIVMTQSPLSLPVTPGEPASISCRSSQSLLHSNGYNYLDWYLQKPGQSPQLLIYLGSYRASGVPDRFSGSGSGTDFTLKISRVEAEDVGVYYCRQALRTPLTFGGGTKVEIK

SEQ ID NO: 110 - protein, 121 aa, synthetic construct
QVQLVQSGAEVKKPGASVKVSCKASGYTFTSYDINWVRQAPGQGLEWMGWISGYNGNTNYAQKFQGRVTMTRDTSTSTVYMELSSLRSEDTAVYYCARSHEYYYYYGMDVWGQGTLVTVSS

SEQ ID NO: 111 - protein, 108 aa, synthetic construct
EIVMTQSPATLSVSPGERATLSCRASQSVSSSYLAWYQQKPGQAPRLLIYGASTRATGIPARFSGSGSGTEFTLTISSLQSEDFAVYYCQQYYTTPFTFGPGTKVDIK

SEQ ID NO: 112 - protein, 129 aa, synthetic construct
EVQLLESGGGLVQPGGSLRLSCAASGFTFSSYDMHWVRQAPGKGLEWVSAISSSGGSIYYADSVKGRFTISRDNSKNTLYLQMNSLRAEDTAVYYCARDSVVVPKGPNRKYYYYGMDVWGQGTKVTVSS

SEQ ID NO: 113 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQATHSPYTFGQGTKVEIK

SEQ ID NO: 114 - protein, 126 aa, synthetic construct
EVQLLESGGGLVQPGGSLRLSCAASGFTFDAYAMHWVRQAPGKGLEWVSAIGTGGDTYYADSVKGRFTISRDNSKNTLYLQMNSLRAEDTAVYYCARDLYGSGSPQYYYYYGMDVWGQGTTVTVSS

SEQ ID NO: 115 - protein, 107 aa, synthetic construct
DIQMTQSPSSLSASVGDRVTITCRASQGIKNDLGWYQQKPGKAPKLLIYAASSLQSGVPSRFSGSGSGTDFTLTISSLQPEDFATYYCQQSYSVPFTFGQGTKVEIK

SEQ ID NO: 116 - protein, 121 aa, synthetic construct
QVQLVQSGAEVKKPGASVKVSCKASGYAFTAYYLHWVRQAPGQGLEWMGWMNPNSGNTGYAQKFQGRVTMTRDTSTSTVYMELSSLRSEDTAVYYCAREGWGYYDGGFDPWGQGTLVTVSS

SEQ ID NO: 117 - protein, 112 aa, synthetic construct
DIVMTQSPLSLPVTPGEPASISCRSSQSLLHSNGYNYLDWYLQKPGQSPQLLIYLGSRRASGVPDRFSGSGSGTDFTLKISRVEAEDVGVYYCMQGSHWPPTFGPGTKVDIK

SEQ ID NO: 118 - protein, 128 aa, synthetic construct
QVQLVQSGAEVKKPGASVKVSCKASGGTSSTYAVTWVRQAPGQGLEWMGVINPNDGSTTYAQNFQGRVTMTRDTSTSTVYMELSSLRSEDTAVYYCARWTPLFGVVIPDYYYYGMDVWGQGTLVTVSS

SEQ ID NO: 119 - protein, 107 aa, synthetic construct
DIQMTQSPSSLSASVGDRVTITCQASQDISNYLNWYQQKPGKAPKLLIYAASSLQSGVPSRFSGSGSGTDFTLTISSLQPEDFATYYCQQSYSTPPTFGQGTRLEIK

SEQ ID NO: 120 - protein, 115 aa, synthetic construct
QVQLVQSGAEVKKPGASVKVSCKASGGTFSSYVISWVRQAPGQGLEWMGWMNPGSGNTGYAQKFQGRVTMTRDTSTSTVYMELSSLRSEDTAVYYCITDSEDDYWGQGTLVTVSS

SEQ ID NO: 121 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYNTPYTFGQGTKVEIK

SEQ ID NO: 122 - protein, 128 aa, synthetic construct
QVQLVQSGAEVKKPGASVKVSCKASGHTFTSQYMHWVRQAPGQGLEWVGVINPNDGSTSYAQKFQGRVTMTRDTSTSTVYMELSSLRSEDTAVYYCARDGAYYYDSSGYYRSSNFDYWGQGTLVTVSS

SEQ ID NO: 123 - protein, 107 aa, synthetic construct
DIQMTQSPSSLSASVGDRVTITCQASQDIGNYLNWYQQKPGKAPKLLIYGASNLETGVPSRFSGSGSGTDFTLTISSLQPEDFATYYCQQGYSFPLTFGQGTKVEIK

SEQ ID NO: 124 - protein, 119 aa, synthetic construct
QVQLVQSGAEVKKPGASVKVSCKASGYTFTSNYMHWVRQAPGQGLEWMGWMNPNSGNTAYAQKFQGRVTMTRDTSTSTVYMELSSLRSEDTAVYYCARGRGGQLLFDYWGQGTLVTVSS

SEQ ID NO: 125 - protein, 106 aa, synthetic construct
DIQMTQSPSSLSASVGDRVTITCRASQGISSWLAWYQQKPGKAPKLLIYAASTLQSGVPSRFSGSGSGTDFTLTISSLQPEDFATYYCQQYNSYSTFGQGTKVEIK

SEQ ID NO: 126 - protein, 121 aa, synthetic construct
QVQLVQSGAEVKKPGASVKVSCKASGGTFSNYGISWVRQAPGQGLEWMGIINPTDGSTTYAQKFQGRVTMTRDTSTSTVYMELSSLRSEDTAVYYCARWWGSGWSWYFDLWGRGTLVTVSS

SEQ ID NO: 127 - protein, 107 aa, synthetic construct
DIQMTQSPSSLSASVGDRVTITCRASQSISSYLNWYQQKPGKAPKLLIYAASSLESGVPSRFSGSGSGTDFTLTISSLQPEDFATYYCQQSYSTPPTFGQSTRLEIK

SEQ ID NO: 128 - protein, 116 aa, synthetic construct
QVQLVQSGAEVKKPGASVKVSCKASGYTFTSYAINWVRQAPGQGLEWMGILSPSGGGTSYAPKFQGRVTMTRDTSTSTVYMELSSLRSEDTAVYYCARGPWYFDLWGRGTLVTVSS

SEQ ID NO: 129 - protein, 107 aa, synthetic construct
DIQMTQSPSSLSASVGDRVTITCRASQGISNSLAWYQQKPGKAPKLLIYAASSLQSGVPSRFSGSGSGTDFTLTISSLQPEDFATYYCQQSYSTPFTFGPGTKVDIK

SEQ ID NO: 130 - protein, 119 aa, synthetic construct
QVQLVQSGAEVKKPGSSVKVSCKASGGTFSSYAISWVRQAPGQGLEWMGGIVPMLGSPHYAQKFQGRVTITADESTSTAYMELSSLRSEDTAVYYCARGSWLVADFQHWGQGTLVTVSS

SEQ ID NO: 131 - protein, 113 aa, synthetic construct
DIVMTQSPDSLAVSLGERATINCKSSQSLLYSSNNRNYLAWYQQKPGQPPKLLIYWASTRESGVPDRFSGSGSGTDFTLTISSLQAEDVAVYYCQQYYSTPITFGQGTRLEIK

SEQ ID NO: 132 - protein, 121 aa, synthetic construct
EVQLLESGGGLVKPGGSLRLSCAASGFRFSVYWMSWVRQAPGKGLEWVSGISGSGGTTYYADSVKGRFTISRDDSKNTLYLQMNSLKTEDTAVYYCARVRRDGYNYNFDYWGQGTLVTVSS

SEQ ID NO: 133 - protein, 107 aa, synthetic construct
DIQMTQSPSSLSASVGDRVTITCQASQDISNYLNWYQQKPGKAPKLLIFDASSLEAGVPSRFSGSGSGTDFTLTISSLQPEDFATYYCQQANSFPPTFGQGTEVEIK

SEQ ID NO: 134 - protein, 124 aa, synthetic construct
QVQLVQSGAEVKKPGSSVKVSCKASGFTFTTSAVQWVRQAPGQGLEWMGGIIPIFGTANYAQKFQGRVTITADESTSTAYMELSSLRSEDTAVYYCAKGSGYEFPGGSEYFQHWGQGTLVTVSS

SEQ ID NO: 135 - protein, 113 aa, synthetic construct
DIVMTQSPDSLAVSLGERATINCKSSQSVLYNSNNLNYLAWYQQKPGQPPKLLIYWASIRESGVPDRFSGSGSGTDFTLTISSLQAEDVAVYYCQQYYSTPLTFGQGTKVEIK

SEQ ID NO: 136 - protein, 129 aa, synthetic construct
QVQLVQSGAEVKKPGASVKVSCKASGGTLSSYAISWVRQAPGQGLEWMGWIDPNSGGTNYAQKFQGRVTMTRDTSTSTVYMELSSLRSEDTAVYYCARQGLLWFGESGSIYYYYGMDVWGQGTTVTVSS

SEQ ID NO: 137 - protein, 107 aa, synthetic construct
DIQMTQSPSSLSASVGDRVTITCRASRSISNYLNWYQQKPGKAPKLLIYGASSLQSGVPSRFSGSGSGTDFTLTISSLQPEDFATYYCQQSYSTLVTFGQGTKVEIK

SEQ ID NO: 138 - protein, 123 aa, synthetic construct
QVQLVQSGAEVKKPGASVKVSCKASGYTFTSYGISWVRQAPGQGLEWMGWMNPNSGNTGYAQKFQGRVTMTRDTSTSTVYMELSSLRSEDTAVYYCARLGLWFGEYQYYFDYWGQGTLVTVSS

SEQ ID NO: 139 - protein, 113 aa, synthetic construct
DIVMTQSPDSLAVSLGERATINCKSSQSVLYSSNNKNYLAWYQQKPGQPPKLLIYWASTREPGVPDRFSGSGSGTDFTLTISSLQAEDVAVYYCQQYYSTPLTFGQGTKVEIK

SEQ ID NO: 140 - protein, 122 aa, synthetic construct
EVQLLESGGGLVKPGGSLRLSCAASGFTFSSYWMSWVRQAPGKGLEWVSGISWNGGTVGYADSVKGRFTISRDDSKNTLYLQMNSLKTEDTAVYYCAKLGIAVKSHWYFDLWGRGTLVTVSS

SEQ ID NO: 141 - protein, 107 aa, synthetic construct
DIQMTQSPSSLSASVGDRVTITCRASESISTWLAWYQQKPGKAPKLLIYAASSLQSGVPSRFSGSGSGTDFTLTISSLQPEDFATYYCQQSYTLPLTFGGGTKLEIK

SEQ ID NO: 142 - protein, 123 aa, synthetic construct
QVQLVQSGAEVKKPGASVKVSCKASGYTFTSYDINWVRQAPGQGLEWVGIINPSGGSTSYAQKFQGRVTMTRDTSTSTVYMELSSLRSEDTAVYYCARASSGGYYYYYGMDVWGQGTTVTVSS

SEQ ID NO: 143 - protein, 107 aa, synthetic construct
DIQMTQSPSSLSASVGDRVTITCRASQDIVNWLAWYQQKPGKAPKLLIYAASSLQSGVPSRFSGSGSGTDFTLTISSLQPEDFATYYCQQYYSYPLTFGQGTRLEIK

SEQ ID NO: 144 - protein, 120 aa, synthetic construct
QVQLVQSGAEVKKPGASVKVSCKASGGTFSNYAISWVRQAPGQGLEWMGVINPRGGSTTYAQKFQGRVTMTRDTSTSTVYMELSSLRSEDTAVYYCARDYSIPYYGMDVWGQGTTVTVSS

SEQ ID NO: 145 - protein, 107 aa, synthetic construct
DIQMTQSPSSLSASVGDRVTITCRASQSISTYLNWYQQKPGKAPKLLIYDASNLETGVPSRFSGSGSGTDFTLTISSLQPEDFATYYCQQSYSTPLTFGGGTKVEIK

SEQ ID NO: 146 - protein, 117 aa, synthetic construct
EVQLLESGGGLVQPGGSLRLSCAASGFTFSNHYMSWVRQAPGKGLEWVAVIALDGSYRYYADSVKGRFTISRDNSKNTLYLQMNSLRAEDTAVYYCARVGPGGMDVRGQGTTVTVSS

SEQ ID NO: 147 - protein, 109 aa, synthetic construct
DIQMTQSPSSLSASVGDRVTITCQASQDISNYLNWYQQKPGKAPKLLIYAASSLQSGVPSRFSGSGSGTDFTLTISSLQPEDFATYYCQQSYNSPRVYTFGQGTKVEIK

SEQ ID NO: 148 - protein, 120 aa, synthetic construct
QVQLVQSGAEVKKPGASVKVSCKASGGRFSTYALSWVRQAPGQGLEWMGIINPTDGSTTYAQKFQGRVTMTRDTSTSTVYMELSSLRSEDTAVYYCARDVYSSSWYSDYWGQGTLVTVSS

SEQ ID NO: 149 - protein, 107 aa, synthetic construct
DIQMTQSPSSLSASVGDRVTITCRASQSISNWLAWYQQKPGKAPKLLIYAASTLQSGVPSRFSGSGSGTDFTLTISSLQPEDFATYYCQQSYSVPFTFGPGTKVDIK

SEQ ID NO: 150 - protein, 116 aa, synthetic construct
EVQLLESGGGLVQPGGSLRLSCAASGFPFSTYAIHWVRQAPGKGLEWVSAISGSGGSTYYADSVKGRFTISRDNSKNTLYLQMNSLRAEDTAVYYCAGPDWYFDLWGRGTLVTVSS

SEQ ID NO: 151 - protein, 107 aa, synthetic construct
DIQMTQSPSSLSASVGDRVTITCRASQAISNSLAWYQQKPGKAPKLLIYAASTLQRGVPSRFSGSGSGTDFTLTISSLQPEDFATYYCQQYYSTPYTFGQGTKLEIK

SEQ ID NO: 152 - protein, 126 aa, synthetic construct
QVQLVQSGAEVKKPGASVKVSCKASGGTFNNYAINWVRQAPGQGLEWMGTINPRDGDTTYAQKFQGRVTMTRDTSTSTVYMELSSLRSEDTAVYYCARVGYYGSGSYYSYYGMDVWGQGTTVTVSS

SEQ ID NO: 153 - protein, 107 aa, synthetic construct
DIQMTQSPSSLSASVGDRVTITCRASQNITNWLAWYQQKPGKAPKLLIYAASSLQSGVPSRFSGSGSGTDFTLTISSLQPEDFATYYCQQAYSFPWTFGQGTKVEIK

SEQ ID NO: 154 - protein, 124 aa, synthetic construct
QVQLVQSGAEVKKPGASVKVSCKASGGTFSSYAISWVRQAPGQGLEWMGWVSGYNGNANYAQKFQGRVTMTRDTSTSTVYMELSSLRSEDTAVYYCARDRVESGYSYHDAFDIWGQGTMVTVSS

SEQ ID NO: 155 - protein, 107 aa, synthetic construct
DIQMTQSPSSLSASVGDRVTITCRTSQSIRRYLNWYQQKPGKAPKLLIYAASSLQSGVPSRFSGSGSGTDFTLTISSLQPEDFATYYCQQSYSIPWTFGPGTKVDIK

SEQ ID NO: 156 - protein, 119 aa, synthetic construct
QVQLVQSGAEVKKPGASVKVSCKASGYTFTSYGISWVRQAPGQGLEWMGWMNPNNGNTVYAQKFQGRVTMTRDTSTSTVYMELSSLRSEDTAVYYCAKDSDWYGAFDIWGQGTMVTVSS

SEQ ID NO: 157 - protein, 107 aa, synthetic construct
DIQMTQSPSSLSASVGDRVTITCQASQSISRWLAWYQQKPGKAPKLLIYSASSLQSGVPSRFSGSGSGTDFTLTISSLQPEDFATYYCQQSYSTPWTFGQGTKLEIK

SEQ ID NO: 158 - protein, 124 aa, synthetic construct
QVQLVQSGAEVKKPGASVKVSCKASGYTFTSYDINWVRQAPGQGLEWIGWMNTNSGDTGYAQKFQGRVTMTRDTSTSTVYMELSSLRSEDTAVYYCARQAYSSSWYWYGWFDPWGQGTLVTVSS

SEQ ID NO: 159 - protein, 107 aa, synthetic construct
DIQMTQSPSSLSASVGDRVTITCQASHDIDNYLNWYQQKPGKAPKLLIYAASNLQSGVPSRFSGSGSGTDFTLTISSLQPEDFATYYCQQANSFPLTFGPGTKVDIK

SEQ ID NO: 160 - protein, 118 aa, synthetic construct
QVQLVQSGAEVKKPGASVKVSCKASGYTFPNYGITWVRQAPGQGLEWMGWMNPNSGYTGYAQNFQGRVTMTRDTSTSTVYMELSSLRSEDTAVYYCARGMPGRGFDPWGQGTLVTVSS

SEQ ID NO: 161 - protein, 108 aa, synthetic construct
EIVMTQSPATLSVSPGERATLSCRASQSVSSNYLAWYQQKPGQAPRLLIYGASTRATGIPARFSGSGSGTEFTLTISSLQSEDFAVYYCQQYHTYPPTFGQGTKLEIK

SEQ ID NO: 162 - protein, 121 aa, synthetic construct
QVQLVQSGAEVKKPGASVKVSCKASGYTFSSDYIHWVRQAPGQGLEWMGRINPSGGSTLYARRFQGRVTMTRDTSTSTVYMELSSLRSEDTAVYYCARERGAADTWYFDLWGRGTLVTVSS

SEQ ID NO: 163 - protein, 107 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYRTPLTFGGGTKVEIK

SEQ ID NO: 164 - protein, 118 aa, synthetic construct
QVQLVQSGAEVKKPGASVKVSCKASGGTFSSYAISWVRQAPGQGLEWMGIIDPTGGATAYAQEFQGRVTMTRDTSTSTVYMELSSLRSEDTAVYYCARVGYYYGMDVWGQGTMVTVSS

SEQ ID NO: 165 - protein, 107 aa, synthetic construct
DIQMTQSPSSLSASVGDRVTITCRASQSVSTYLNWYQQKPGKAPKLLIYAASSLQSGVPSRFSGSGSGTDFTLTISSLQPEDFATYYCQQSYSTPLTFGGGTKVEIK

SEQ ID NO: 166 - protein, 120 aa, synthetic construct
QVQLVQSGAEVKKPGSSVKVSCKASGGTFSSYAISWVRQAPGQGLEWMGGIIPISSATSIPQKFQGRVTITADESTSTAYMELSSLRSEDTAVYYCARSYDSRYYGMDVWGQGTTVTVSS

SEQ ID NO: 167 - protein, 108 aa, synthetic construct
EIVMTQSPATLSVSPGERATLSCRASQTVGSRYLAWYQQKPGQAPRLLIYGASTRATGIPARFSGSGSGTEFTLTISSLQSEDFAVYYCQQYYSTPWTFGQGTRLEIK

SEQ ID NO: 168 - protein, 116 aa, synthetic construct
QVQLVQSGAEVKKPGASVKVSCKASGGTFSSYAISWVRQAPGQGLEWMGWMNPNSGDTGYAQKFQGRVTMTRDTSTSTVYMELSSLRSEDTAVYYCARGPWYFDLWGRGTLVTVSS

SEQ ID NO: 169 - protein, 107 aa, synthetic construct
DIQMTQSPSSLSASVGDRVTITCRVSQGISNSLAWYQQKPGKAPKLLIYAASSLQSGVPSRFSGSGSGTDFTLTISSLQPEDFATYYCQQSYSTVYTFGQGTKLEIK

SEQ ID NO: 170 - protein, 116 aa, synthetic construct
EVQLLESGGGLVKPGGSLRLSCAASGFMFSSYSMNWVRQAPGKGLEWVSYISGNSGYTNYADSVKGRFTISRDDSKNTLYLQMNSLKTEDTAVYYCARRLHGMDVWGQGTTVTVSS

SEQ ID NO: 171 - protein, 107 aa, synthetic construct
DIQMTQSPSSLSASVGDRVTITCRASQSISTWLAWYQQKPGKAPKLLIYDASNLETGVPSRFSGSGSGTDFTLTISSLQPEDFATYYCQQAYRFPVAFGGGTKVEIK

SEQ ID NO: 172 - protein, 118 aa, synthetic construct
EVQLLESGGGLVQPGGSLRLSCAASGFTFSSYWMHWVRQAPGKGLEWVSVISNSGGATYYADSVKGRFTISRDNSKNTLYLQMNSLRAEDTAVYYCAREGWGYGMDVWGQGTTVTVSS

SEQ ID NO: 173 - protein, 107 aa, synthetic construct
DIQMTQSPSSLSASVGDRVTITCRASQDIRNYLAWYQQKPGKAPKLLIYTASTLQSGVPSRFSGSGSGTDFTLTISSLQPEDFATYYCQQTFRTPLTFGGGTKVEIK

SEQ ID NO: 174 - protein, 122 aa, synthetic construct
QVQLVQSGAEVKKPGASVKVSCKASGYTFTNYYMHWVRQAPGQGLEWMGLITPSGGRTKYAQKFQGRVTMTRDTSTSTVYMELSSLRSEDTAVYYCAREMEYSSSWYWFDPWGQGTLVTVSS

SEQ ID NO: 175 - protein, 107 aa, synthetic construct
DIQMTQSPSSLSASVGDRVTITCQASQGISSYLNWYQQKPGKAPKLLIYAASTLQSGVPSRFSGSGSGTDFTLTISSLQPEDFATYYCQQSYSTPLTFGGGTKVEIK

SEQ ID NO: 176 - protein, 123 aa, synthetic construct
QVQLVQSGAEVKKPGASVKVSCKASGYTFTRNYIHWVRQAPGQGLEWMGWINPKSGGTKYAQKFQGRVTMTRDTSTSTVYMELSSLRSEDTAVYYCARESGVVATEYWYFDLWGRGTLVTVSS

SEQ ID NO: 177 - protein, 107 aa, synthetic construct [sequence omitted]

SEQ ID NO: 178 - protein, 127 aa, synthetic construct
QVQLVQSGAEVKKPGASVKVSCKASGYTFTSYYMHWVRQAPGQGLEWMGWINPNSGGTNYAQKFQGRVTMTRDTSTSTVYMELSSLRSEDTAVYYCASPGDYCSGGSCYSDDAFDIWGQGTMVTVSS

SEQ ID NO: 179 - protein, 113 aa, synthetic construct
DIVMTQSPDSLAVSLGERATINCKSSQSIFYSSNSKNYLAWYQQKPGQPPKLLIYWASTRESGVPDRFSGSGSGTDFTLTISSLQAEDVAVYYCQQYYSTPLTFGPGTKVDIK

SEQ ID NO: 180 - protein, 122 aa, synthetic construct
QVQLVQSGAEVKKPGASVKVSCKASGGTFRNYAINWVRQAPGQGLEWMGWMNPNSGNTGYAQKFQGRVTMTRDTSTSTVYMELSSLRSEDTAVYYCAREDVDTASQAYFDYWGQGTLVTVSS

SEQ ID NO: 181 - protein, 107 aa, synthetic construct
DIQMTQSPSSLSASVGDRVTITCRASQGISNYLAWYQQKPGKAPKLLIYSASNLQSGVPSRFSGSGSGTDFTLTISSLQPEDFATYYCQQGYSSRYTFGQGTKLEIK

SEQ ID NO: 182 - protein, 120 aa, synthetic construct
EVQLLESGGGLVKPGGSLRLSCAASGFTFSSYWMSWVRQAPGKGLEWVSSISWSSTYIYYADSVKGRFTISRDDSKNTLYLQMNSLKTEDTAVYYCARDGQLGHWHFDLWGRGTLVTVSS

SEQ ID NO: 183 - protein, 107 aa, synthetic construct
DIQMTQSPSSLSASVGDRVTITCRASQSISDWLAWYQQKPGKAPKLLIYEASKLATGVPSRFSGSGSGTDFTLTISSLQPEDFATYYCQQSYTTPLTFGGGTKVEIK

SEQ ID NO: 184 - protein, 117 aa, synthetic construct
EVQLLESGGGLVQPGGSLRLSCAASGFTFRNHWMSWVRQAPGKGLEWVSGISWNSGSIDYADSVKGRFTISRDNSKNTLYLQMNSLRAEDTAVYYCAREEYATFDYWGQGTLVTVSS

SEQ ID NO: 185 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPTFGPGTKVDIK

SEQ ID NO: 186 - protein, 122 aa, synthetic construct
QVQLVQSGAEVKKPGASVKVSCKASGGSFSDYAVSWVRQAPGQGLEWMGWMNPNSGNTGYAQKFQGRVTMTRDTSTSTVYMELSSLRSEDTAVYYCARELVRDGYNFALDYWGQGTLVTVSS

SEQ ID NO: 187 - protein, 107 aa, synthetic construct
DIQMTQSPSSLSASVGDRVTITCRASQDIRNDLAWYQQKPGKAPKLLIYAASSLQSGVPSRFSGSGSGTDFTLTISSLQPEDFATYYCQQSFSTFYTFGQGTKLEIK

SEQ ID NO: 188 - protein, 117 aa, synthetic construct
QVQLVQSGAEVKKPGASVKVSCKASGYIFTNYWIQWVRQAPGQGLEWMGWINPHSGATKYAERFQGRVTMTRDTSTSTVYMELSSLRSEDTAVYYCARGLGSAFDIWGQGTMVTVSS

SEQ ID NO: 189 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 190 - protein, 126 aa, synthetic construct
QVQLVQSGAEVKKPGASVKVSCKASGYTFTSHDIDWVRQAPGQGLEWMGWMNPNNGNTVYAQRFQGRVTMTRDTSTSTVYMELSSLRSEDTAVYYCARWKVYSGSYYGGAGYFDLWGRGTLVTVSS

SEQ ID NO: 191 - protein, 107 aa, synthetic construct
DIQMTQSPSSLSASVGDRVTITCRASQSISSYLNWYQQKPGKAPKLLIYRASHLEGGVPSRFSGSGSGTDFTLTISSLQPEDFATYYCQQADSLPLTFGQGTKVEIK

SEQ ID NO: 192 - protein, 120 aa, synthetic construct
QVQLVQSGAEVKKPGASVKVSCKASGYTFTSHDIDWVRQAPGQGLEWMGWINPSGDSTSYAQKFQGRVTMTRDTSTSTVYMELSSLRSEDTAVYYCARYYGGNSYAFDIWGQGTMVTVSS

SEQ ID NO: 193 - protein, 107 aa, synthetic construct
DIQMTQSPSSLSASVGDRVTITCRASESISPWVAWYQQKPGKAPKLLIYAASTLQSGVPSRFSGSGSGTDFTLTISSLQPEDFATYYCQQGTSTPYTFGQGTKLEIK

SEQ ID NO: 194 - protein, 117 aa, synthetic construct
QVQLVQSGAEVKKPGASVKVSCKASGYTFTSYGISWVRQAPGQGLEWMGWMNPNSGNTGYAQKFQGRVTMTRDTSTSTVYMELSSLRSEDTAVYYCAREVYGDLDYWGQGTLVTVSS

SEQ ID NO: 195 - protein, 107 aa, synthetic construct
DIQMTQSPSSLSASVGDRVTITCRASQGIDNWLAWYQQKPGKAPKLLIYGASTLQSGVPSRFSGSGSGTDFTLTISSLQPEDFATYYCQQSYTARFTFGPGTKVDIK

SEQ ID NO: 196 - protein, 121 aa, synthetic construct
QVQLVQSGAEVKKPGASVKVSCKASGGSFSNYAINWVRQAPGQGLEWMGWMNPYSGNTGYAQKFQGRVTMTRDTSTSTVYMELSSLRSEDTAVYYCAREITANYYYGMDVWGQGTKVTVSS

SEQ ID NO: 197 - protein, 107 aa, synthetic construct
DIQMTQSPSSLSASVGDRVTITCQASQDISNYLNWYQQKPGKAPKLLIYEASVLESGVPSRFSGSGSGTDFTLTISSLQPEDFATYYCQQSYTTQYTFGQGTKVEIK

SEQ ID NO: 198 - protein, 119 aa, synthetic construct
QVQLVQSGAEVKKPGSSVKVSCKASGGTFSTYAINWVRQAPGQGLEWMGIINPTDGDTSYAQKFQGRVTITADESTSTAYMELSSLRSEDTAVYYCARAAYYYYGMDVWGQGTTVTVSS

SEQ ID NO: 199 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALHIPYTFGQGTKLEIK

SEQ ID NO: 200 - protein, 125 aa, synthetic construct
QVQLVQSGAEVKKPGASVKVSCKASGYSFTSHDIDWVRQAPGQGLEWMGWINPNSGGTNYAQKFQGRVTMTRDTSTSTVYMELSSLRSEDTAVYYCARIRGYYGSGSYHDAFDIWGQGTTVTVSS

SEQ ID NO: 201 - protein, 107 aa, synthetic construct
DIQMTQSPSSLSASVGDRVTITCRASQTISTYLNWYQQKPGKAPKLLIYAASSLQSGVPSRFSGSGSGTDFTLTISSLQPEDFATYYCQQSYSTPWTFGQGTKVEIK

SEQ ID NO: 202 - protein, 116 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 203 - protein, 108 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 204 - protein, 119 aa, synthetic construct
QVQLVQSGAEVKKPGSSVKVSCKASGGTFNSYAISWVRQAPGQGLEWMGGIIPGFGTANYAQKFQGRVTITADESTSTAYMELSSLRSEDTAVYYCARKNEEDGGFDHWGQGTLVTVSS

SEQ ID NO: 205 - protein, 107 aa, synthetic construct
DIELTQPPSVSVAPGQTARISCSGDNLGDYYASWYQQKPGQAPVLVIYDDSNRPSGIPERFSGSNSGNTATLTISGTQAEDEADYYCQTWDGTLHFVFGGGTKLTVL

SEQ ID NO: 206 - protein, 118 aa, synthetic construct
QVQLVESGGGLVQPGGSLRLSCAASGFTFSDYYMHWVRQAPGKGLEWVSVISGSGSNTYYADSVKGRFTISRDNSKNTLYLQMNSLRAEDTAVYYCARLYAQFEGDFWGQGTLVTVSS

SEQ ID NO: 207 - protein, 107 aa, synthetic construct
DIELTQPPSVSVAPGQTARISCSGDNIGSKYVSWYQQKPGQAPVLVIYSDSERPSGIPERFSGSNSGNTATLTISGTQAEDEADYYCQSWDGSISRVFGGGTKLTVL

SEQ ID NO: 208 - protein, 116 aa, synthetic construct
QVQLVQSGAEVKKPGESLKISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 209 - protein, 108 aa, synthetic construct
DIELTQPPSVSVAPGQTARISCSGDNIGDQYAHWYQQKPGQAPVVVIYQDKNRPSGIPERFSGSNSGNTATLTISGTQAEDEADYYCATYTGFGSLAVFGGGTKLTVL

SEQ ID NO: 210 - protein, 108 aa, synthetic construct
DIELTQPPSVSVAPGQTARISCSGDNIGDQYAHWYQQKPGQAPVVVIYQDKNRPSGIPERFSGSNSGNTATLTISGTQAEDEADYYCSTYTFVGFTTVFGGGTKLTVL

SEQ ID NO: 211 - protein, 119 aa, synthetic construct
QVQLQQSGAEVKKPGASVKLSCKASGYTFSSYWMHWVRQAPGQGLEWIGEINPGNGHTNYNEKFKSRATMTRDTSTSTAYMELSSLRSEDSAVYYCARSFTTARAFAYWGQGTLVTVSS

SEQ ID NO: 212 - protein, 107 aa, synthetic construct
DIVMTQSPAFLSVTPGEKVTITCRASQTISDYLHWYQQKPDQAPKLLIKYASQSISGIPSRFSGSGSGTDFTFTISSLEAEDAATYYCQDGHSWPPTFGQGTKLEIK

SEQ ID NO: 213 - protein, 119 aa, synthetic construct
QVQLQQSGAEVKKPGASVKLSCKASGYTFSSYWMHWVRQAPGQGLEWIGEINPGNGHTNYNEKFKSRATMTRDTSTSTAYMELSSLRSEDSAVYYCARSFKTARAFAYWGQGTLVTVSS

SEQ ID NO: 214 - protein, 119 aa, synthetic construct
QVQLVQSGAEVKKPGASVKLSCKASGYTFSSYWMHWVRQAPGQGLEWIGEINPGNGHTNYNEKFKSRVTMTRDTSTSTAYMELSSLRSEDSAVYYCARSFKTARAFAYWGQGTLVTVSS

SEQ ID NO: 215 - protein, 116 aa, synthetic construct
EVRLQQSGADLVKPGASVKLSCTASGFIIKATYMHWVRQRPEQGLEWIGRIDPANGETKYDPKFQVKATITADTSSSTAYLQLNSLTSDDTAVYYCARYAWYFDVWGAGTTVTVSS

SEQ ID NO: 216 - protein, 107 aa, synthetic construct
DIQMTQSPASLSASVGDTVTITCRASENIYSFLAWYHQKQGRSPQLLVYHAKTLAEGVPSRFSGSGSGTQFSLKINSLQAEDFGSYYCQHYYGSPLTFGAGTKLEVK

SEQ ID NO: 217 - protein, 119 aa, synthetic construct
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 218 - protein, 107 aa, synthetic construct
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 219 - protein, 116 aa, synthetic construct
QIQLVQSGPELKKPGETVKISCKASGYTFTNYGMNWVKQAPGKGLKWMGWINTNTGEPTYAEEFKGRFAFSLETSATTAYLQINNLKNEDTATYFCAREGDAMDYWGQGTSVTVSS

SEQ ID NO: 220 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWNSYPFTFGSGTKLEIK

SEQ ID NO: 221 - protein, 116 aa, synthetic construct
  VARIANT 50: note = Any amino acid
  VARIANT 52: note = Any amino acid
  VARIANT 53: note = Any amino acid
  VARIANT 54: note = Any amino acid
  VARIANT 55: note = Any amino acid
  VARIANT 101: note = Any amino acid
  VARIANT 102: note = Any amino acid
  VARIANT 103: note = Any amino acid
EIQLVQSGAEVKKPGASVKVSCKASGYTFTNYGMNWVKQAPGQGLKWMGXIXXXXGEPTYAEEFKGRFTFTLDTSISTAYMELSSLRSEDTAVYYCAREGXXXDYWGQGTTVTVSS

SEQ ID NO: 222 - protein, 106 aa, synthetic construct
  VARIANT 46: note = Any amino acid
  VARIANT 49: note = Any amino acid
  VARIANT 50: note = Any amino acid
  VARIANT 90: note = Any amino acid
  VARIANT 91: note = Any amino acid
  VARIANT 92: note = Any amino acid
EIVLTQSPATLSLSPGERATLSCSASSSVSYMHWYQQKPGQAPKRXIYXXSKLASGVPARFSGSGSGTDYSLTISSLEPEDFAVYYCQQXXXYPFTFGQGTKLEIK

SEQ ID NO: 223 - protein, 116 aa, synthetic construct
  VARIANT 50: note = Any amino acid
  VARIANT 52: note = Any amino acid
  VARIANT 53: note = Any amino acid
  VARIANT 54: note = Any amino acid
  VARIANT 55: note = Any amino acid
  VARIANT 101: note = Any amino acid
  VARIANT 102: note = Any amino acid
  VARIANT 103: note = Any amino acid
EIQLVQSGAEVKKPGASVKVSCKASGYTFTNYGMNWVKQAPGQGLKWMGXIXXXXGEPTYAEEFKGRFAFTLDTSISTAYMELSSLRSEDTAVYYCAREGXXXDYWGQGTTVTVSS

SEQ ID NO: 224 - protein, 106 aa, synthetic construct
  VARIANT 46: note = Any amino acid
  VARIANT 49: note = Any amino acid
  VARIANT 50: note = Any amino acid
  VARIANT 90: note = Any amino acid
  VARIANT 91: note = Any amino acid
  VARIANT 92: note = Any amino acid
EIVLTQSPATLSLSPGEKATLSCSASSSVSYMHWYQQKPGQAPKRXIYXXSKLASGVPARFSGSGSGTDYSLTISSLEAEDFAVYYCQQXXXYPFTFGQGTKLEIK

SEQ ID NO: 225 - protein, 116 aa, synthetic construct
EIQLVQSGAEVKKPGASVKVSCKASGYTFTNYGMNWVKQAPGQGLKWMGWINTNTGEPTYAEEFKGRFAFTLDTSISTAYMELSSLRSEDTAVYYCAREGDAMDYWGQGTTVTVSS

SEQ ID NO: 226 - protein, 106 aa, synthetic construct
EIVLTQSPATLSLSPGEKATLSCSASSSVSYMHWYQQKPGQAPKRWIYDTSKLASGVPARFSGSGSGTDYSLTISSLEAEDFAVYYCQQWNSYPFTFGQGTKLEIK

SEQ ID NO: 227 - protein, 116 aa, synthetic construct [sequence omitted]

SEQ ID NO: 228 - protein, 106 aa, synthetic construct
EIVLTQSPATLSLSPGERATLSCSASSSVSYMHWYQQKPGQAPKRWIYDTSKLASGVPARFSGSGSGTDYSLTISSLEPEDFAVYYCQQWNSYPFTFGQGTKLEIK

SEQ ID NO: 229 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 230 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 231 - protein, 117 aa, synthetic construct
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSS

SEQ ID NO: 232 - protein, 111 aa, synthetic construct
EIVLTQSPDSLAVSLGERATINCKASQSVDFDGDSYLNWYQQKPGQPPKVLIYAASTLQSGVPSRFSGSGSGTDFTLTINSLEAEDAATYYCQQSNEDPWTFGGGTKVEIK

SEQ ID NO: 233 - protein, 117 aa, synthetic construct
QIQLVQSGPEVKKPGASVKVSCKASGYTFTDYYITWVRQAPGQGLEWMGWIYPGSGNTKYNEKFQGRFVFSVDTSASTAYLQISSLKAEDTAVYFCANYGNYWFAYWGQGTLVTVSS

SEQ ID NO: 234 - protein, 111 aa, synthetic construct
DIVLTQSPDSLAVSLGERATINCKASQSVDFDGDSYMNWYQQKPGQPPKVLIYAASNLESGIPARFSGSGSGTDFTLTINSLEAEDAATYYCQQSNEDPWTFGGGTKVEIK

SEQ ID NO: 235 - protein, 117 aa, synthetic construct
QLQLVQSGAEVKKPGASVKVSCKVSGYTFTSYYISWVRQAPGQALEWMGWIYAGSGNTKYSQKFQGRFVFSVDTSASTAYLQISSLKAEDTAVYYCANYGNYWFAYWGQGTLVTVSS

SEQ ID NO: 236 - protein, 111 aa, synthetic construct
AIVLTQSPDSLAVSLGERATINCKASQSVDFDGDSYLNWYQQKPGQPPKVLIYAASTLETGVPSRFSGSGSGTDFTLTINSLEAEDAATYYCQQSNEDPWTFGGGTKVEIK

SEQ ID NO: 237 - protein, 117 aa, synthetic construct
QLQLVQSGPEVKKPGASVKVSCKVSGYTFTDYYITWVRQAPGQALEWMGWIYPGSGNTKYNEKFQGRFVFSVDTSASTAYLQISSLKAEDTAVYFCANYGNYWFAYWGQGTLVTVSS

SEQ ID NO: 238 - protein, 117 aa, synthetic construct
QLQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSS

SEQ ID NO: 239 - protein, 120 aa, synthetic construct
MAQVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARLSSGWTFDYWGQGTLVTVSS

SEQ ID NO: 240 - protein, 113 aa, synthetic construct
EIVLTQSPDSLAVSLGERATINCKSSQSVLYSPNKNYLAWYQQKPGQPPKLLIYWASTRESGVPERFSGSGSGTDFTLTISSLHAEDVALYYCQQSYSLPFTFGPGTKVEIKR

SEQ ID NO: 241 - protein, 124 aa, synthetic construct
MAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGYYYGSGGLDYWGQGTLVTVSS

SEQ ID NO: 242 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCLQDYNYPLTFGGGTKVEIK

SEQ ID NO: 243 - protein, 111 aa, synthetic construct
QSALTQPASVSGSPGQSITISCTGTSSDVGAYNYVSWYQQHPGKAPKLMIYEVTKRPSGVPDRFSGSKSGNTASLTISGLQAEDEADYYCISFTASSTWAFGGGTKLTVLG

SEQ ID NO: 244 - protein, 116 aa, synthetic construct
  VARIANT 52: note = Any amino acid
  VARIANT 53: note = Any amino acid
  VARIANT 54: note = Any amino acid
QVQLQESGPGLVKPSETLSLTCTVSGASISSYYWSWIRQPPGKGLEWIGYIXXXGSTNYNPSLKSRVTISVDTSKNQFSLKLRSVTAADTAVYYCARERLGIGDYWGQGTLVTVSS

SEQ ID NO: 245 - protein, 107 aa, synthetic construct
DIQLTQSPSSLSASVGDRVTITCRASQGIRNDLGWYQQKPGKAPKRLIYAASSLQSGVPSRFSGSGSGTEFTLTISSLQPEDFATYYCLQHNTYPPTFGQGTKVEIK

SEQ ID NO: 246 - protein, 116 aa, synthetic construct
QVQLQESGPGLVKPSETLSLTCTVSGASISSYYWSWIRQPPGKGLEWIGYIGGEGSTNYNPSLKSRVTISVDTSKNQFSLKLRSVTAADTAVYYCARERLGIGDYWGQGTLVTVSS

SEQ ID NO: 247 - protein, 116 aa, synthetic construct
QVQLQESGPGLVKPSETLSLTCTVSGASISSYYWSWIRQPPGKGLEWIGYIAGTGSTNYNPSLKSRVTISVDTSKNQFSLKLRSVTAADTAVYYCARERLGIGDYWGQGTLVTVSS

SEQ ID NO: 248 - protein, 116 aa, synthetic construct
QVQLQESGPGLVKPSETLSLTCTVSGASISSYYWSWIRQPPGKGLEWIGYIGYSGSTNYNPSLKSRVTISVDTSKNQFSLKLRSVTAADTAVYYCARERLGIGDYWGQGTLVTVSS

SEQ ID NO: 249 - protein, 116 aa, synthetic construct
QVQLQESGPGLVKPSETLSLTCTVSGASISSYYWSWIRQPPGKGLEWIGYIYGSGSTNYNPSLKSRVTISVDTSKNQFSLKLRSVTAADTAVYYCARERLGIGDYWGQGTLVTVSS

SEQ ID NO: 250 - protein, 116 aa, synthetic construct
QVQLQESGPGLVKPSETLSLTCTVSGASISSYYWSWIRQPPGKGLEWIGYIYYEGSTNYNPSLKSRVTISVDTSKNQFSLKLRSVTAADTAVYYCARERLGIGDYWGQGTLVTVSS

SEQ ID NO: 251 - protein, 116 aa, synthetic construct
QVQLQESGPGLVKPSETLSLTCTVSGASISSYYWSWIRQPPGKGLEWIGYIGGSGSTNYNPSLKSRVTISVDTSKNQFSLKLRSVTAADTAVYYCARERLGIGDYWGQGTLVTVSS

SEQ ID NO: 252 - protein, 116 aa, synthetic construct
QVQLQESGPGLVKPSETLSLTCTVSGASISSYYWSWIRQPPGKGLEWIGYIYGEGSTNYNPSLKSRVTISVDTSKNQFSLKLRSVTAADTAVYYCARERLGIGDYWGQGTLVTVSS

SEQ ID NO: 253 - protein, 116 aa, synthetic construct
QVQLQESGPGLVKPSETLSLTCTVSGASISSYYWSWIRQPPGKGLEWIGYIGYEGSTNYNPSLKSRVTISVDTSKNQFSLKLRSVTAADTAVYYCARERLGIGDYWGQGTLVTVSS

SEQ ID NO: 254 - protein, 116 aa, synthetic construct
DVQLQESGPGLVKPSQSLSLTCTVTGYSITSDYAWNWIRQFPGNKLEWMGYIGYSGRTSFNPSLKSRISITRDTSKNQFFLQLNSVTTEDTATYYCARGGFAMDYWGQGTSVTVSS

SEQ ID NO: 255 - protein, 107 aa, synthetic construct
QIVLTQSPGIMSASPGEKVTMTCSASSSVTYMYWYQQKPRSSPKPWIYLTSNLASGVPARFSGSGSGTSYSLTISSMEAEDGATYYCQQWSSTPPLTFGGGTKLELN

(19) United States
(12) Patent Application Publication
(10) Pub. No.: US 2026/0092123 A1
(43) Pub. Date: Apr. 2, 2026

(54) ENGINEERED IgG MOLECULES AND METHODS OF USE THEREOF

(71) Applicant: Regeneron Pharmaceuticals, Inc., Tarrytown, NY (US)

(72) Inventors: Vivian YEONG, Hillsdale, NJ (US); Yang SHEN, Scarsdale, NY (US); Chia-Yang LIN, Scarsdale, NY (US); Samuel DAVIS, New York, NY (US)

(73) Assignee: Regeneron Pharmaceuticals, Inc., Tarrytown, NY (US)

(21) Appl. No.: 19/345,829

(22) Filed: Sep. 30, 2025

Related U.S. Application Data
(60) Provisional application No. 63/701,847, filed on Oct. 1, 2024.

(51) Int. Cl. C07K 16/28

(52) U.S. Cl. CPC C07K 16/2869; C07K 2317/52; C07K 2317/524; C07K 2317/53; C07K 2317/55; C07K 2317/76; C07K 2317/92

(57) ABSTRACT

The present disclosure relates to engineered IgG molecules having an IgM Cμ2 domain substituted for all or a portion of an IgG hinge region. Engineered IgG molecules may include one or more targeting moieties that are capable of agonizing a cell surface receptor and may be used, for example, in methods of agonizing certain receptors.

### 1. CROSS-REFERENCE TO RELATED APPLICATIONS

This application claims the priority benefit of U.S. provisional application No. 63/701,847, filed on Oct. 1, 2024, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Sep. 24, 2025, is named RGN-048US_SL.xml and is 265,058 bytes in size.

### 3. BACKGROUND

Functional antibodies are an important therapeutic option for treatment of a wide variety of diseases. The binding of some antibodies to a receptor may directly inhibit or activate the receptor, thus regulating cell signaling.

Antibody agonists of multimeric cell membrane receptors exert their agonistic effect in some instances by bringing subunits of the receptor in close proximity to one another, for example when each Fab of an IgG antibody binds to a receptor subunit. Native IgG hinge domains that connect Fab domains to Fc domains allow for a great deal of flexibility. This flexibility can allow receptor subunits bound to the two Fab domains to remain farther apart than would be optimal to achieve the antibody's agonistic effect.

Thus, there exists a need in the art for novel approaches to improve the agonistic activity of antibodies that target cell surface receptors.

### 4. SUMMARY

The present disclosure provides engineered IgG molecules comprising an IgM Cμ2 domain, for example an IgM Cμ2 domain that partially or completely replaces the native IgG hinge region.

Such engineered IgM molecules can have increased rigidity and a more acute angle between IgG Fab domains. Without being bound by theory, the inventors believe that IgG antibodies in which the hinge is partially or completely replaced with an IgM Cμ2 domain have improved agonistic activity when compared to a parental antibody with an IgG hinge due to the capability of Fab domains of the engineered IgG antibodies to bring bound receptor subunits closer to each other than receptor subunits bound by parental antibodies. Exemplary engineered IgG antibodies are disclosed in Section 6.2.4 and numbered embodiments 1 to 170. Exemplary targeting moieties that can be included in engineered IgG molecules of the disclosure are disclosed in Section 6.2.3.

The disclosure further provides nucleic acids encoding the engineered IgG molecules of the disclosure and their components. The nucleic acids can be in the form of a single nucleic acid (e.g., a vector encoding all components of the engineered IgG molecules) or a plurality of nucleic acids (e.g., two or more vectors encoding individual polypeptide chains).

The disclosure further provides host cells and cell lines engineered to express the nucleic acids and the engineered IgG molecules of the disclosure. The disclosure further provides methods of producing an engineered IgG molecule of the disclosure. Exemplary nucleic acids, host cells, cell lines, and methods of producing engineered IgG molecules are described in Section 6.3 and numbered embodiments 171 to 174.

The disclosure further provides pharmaceutical compositions comprising the engineered IgG molecules of the disclosure. Exemplary pharmaceutical compositions are described in Section 6.4 and numbered embodiment 175.

Further provided herein are methods of using the engineered IgG molecules, e.g., for agonizing a target molecule, activating an immune response, or treating a disease or condition associated with or caused by leptin deficiency or leptin resistance. Exemplary methods are described in Section 6.5 and numbered embodiments 176 to 231.

### 5. BRIEF DESCRIPTION OF THE FIGURES

FIGS. 1A-1E illustrate exemplary molecules of the disclosure. FIG. 1A depicts a molecule comprising a first polypeptide chain and a second polypeptide chain, each comprising from N-terminus to C-terminus: an IgM Cμ2 domain, an IgG CH2 domain and an IgG CH3 domain. As disclosed herein, additional components (e.g., an IgG constant domain) may be included N-terminal to the IgM Cμ2 domain on one or both of the polypeptides. FIG. 1B depicts a molecule comprising a first polypeptide chain and a second polypeptide chain, each comprising from N-terminus to C-terminus: an IgG CH1 domain, an IgM Cμ2 domain, an IgG CH2 domain and an IgG CH3 domain. FIG. 1C depicts a molecule comprising a first polypeptide chain and a second polypeptide chain, each comprising from N-terminus to C-terminus: an IgG CL domain, an IgM Cμ2 domain, an IgG CH2 domain and an IgG CH3 domain. FIG. 1D depicts a molecule comprising a first polypeptide chain and a second polypeptide chain, each comprising from N-terminus to C-terminus: a VH domain, an IgG CH1 domain, an IgM Cμ2 domain, an IgG CH2 domain and an IgG CH3 domain. The molecule further comprises a third and a fourth polypeptide chain, each comprising from N-terminus to C-terminus: a VL domain and an IgG CL domain. FIG. 1E depicts a molecule comprising a first polypeptide chain and a second polypeptide chain, each comprising from N-terminus to C-terminus: a VL domain, an IgG CL domain, an IgM Cμ2 domain, an IgG CH2 domain and an IgG CH3 domain. The molecule further comprises a third and a fourth polypeptide molecule, each comprising from N-terminus to C-terminus: a VH domain and an IgG CH1 domain. A molecule as depicted in FIGS. 1A to 1E may have an IgG hinge of reduced length relative to wildtype IgG or may lack an IgG hinge region altogether.

FIGS. 2A-2C are cartoon illustrations of molecules of the disclosure depicting potential modifications to one or more domains. FIG. 2A shows a molecule comprising IgM Cμ2, IgG CH2 and IgG CH3 domains. This molecule includes the amino acids AP at the N-terminus of the IgG CH2 domain generally present in the wildtype sequence. FIG. 2B shows a molecule comprising a modified IgM Cμ2, which has the amino acids PLP added to the N-terminus of the IgM Cμ2 domain (IgM (+PLP) Cμ2). FIG. 2C shows a molecule which has an IgM (+PLP) Cμ2 domain and a modified IgG CH2 domain having the amino acids AP at the N-terminus deleted.

FIG. 3 shows representative negative staining electron microscopy (EM) images of anti-LEPR antibodies either with wild type IgG2 or IgG4 backbones or with IgM Cμ2-IgG2 Fc or IgM Cμ2-IgG4 Fc backbones.

FIGS. 4A-4B show example anti-LEPR antibodies and leptin receptor signaling reporter activation by example anti-LEPR antibodies in the absence of leptin. FIG. 4A is a table that provides descriptions of certain of the anti-LEPR antibodies that were generated.

FIG. 4B is a graph that shows IMR32/STAT3-Luc activity by anti-LEPR(Ab1)-Cμ2-IgG2 antibodies in the absence of leptin in IMR32/STAT3-Luc/hLEPR clone B10 cells.

FIGS. 5A-5B show leptin receptor signaling reporter activation by exemplary anti-LEPR antibodies in the absence or presence of leptin. FIG. 5A is a graph that shows IMR32/STAT3-Luc activity by an anti-LEPR(Ab3)-Cμ2-IgG4 antibody or controls in the absence of leptin in IMR32/STAT3-Luc/hLEPR clone B10 cells. FIG. 5B is a graph that shows IMR32/STAT3-Luc activity by an anti-LEPR(Ab3)-Cμ2-IgG4 antibody or controls in the presence of 1 nM leptin in IMR32/STAT3-Luc/hLEPR clone B10 cells.

FIG. 6 is a graph that shows IMR32/STAT3-Luc activity by an anti-LEPR-Cμ2-IgG4 antibody or controls in the presence of 1 nM leptin in IMR32/STAT3-Luc/hLEPR clone B10 cells.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Aqonistic: The term “agonistic” as used herein with reference to a polypeptide, antibody, or binding molecule refers to the ability to increase signaling, activation, or activity of a protein to which the polypeptide, antibody, or binding molecule is bound.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding Domain: The term “antigen-binding domain” or “ABD” as used herein refers to the portion of a targeting moiety that is capable of specific, non-covalent, and reversible binding to a target molecule.

Associated: The term “associated” in the context of a binding molecule refers to a functional relationship between two or more polypeptide chains or portions of a polypeptide chain. In particular, the term “associated” means that two or more polypeptides are associated with one another, e.g., non-covalently through molecular interactions or covalently through one or more disulfide bridges or chemical cross-linkages, so as to produce a functional binding molecule. Examples of associations that might be present in a binding molecule of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Bivalent: The term “bivalent” as used herein in the context of a binding molecule refers to a binding molecule that has two antigen-binding domains. The domains can be the same or different. Accordingly, a bivalent antigen-binding molecule can be monospecific or bispecific.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABD definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABD numbering scheme); and Lefranc et al., 2003, Dev. Comp. Immunol. 27:55-77 (IMGT numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align. Public databases are available for identifying CDR sequences within an antibody.

EC50: The term “EC50” refers to the half maximal effective concentration of a molecule, such as an antigen-binding molecule, which induces a response halfway between the baseline and maximum after a specified exposure time. The EC50 essentially represents the concentration of a molecule where 50% of its maximal effect is observed. Thus, reduced or weaker binding is observed with an increased EC50, or half maximal effective concentration value.

Epitope: An epitope (or “antigenic determinant”) is a portion of an antigen (e.g., polypeptide antigen) recognized by an antibody or other antigen-binding moiety as described herein. An epitope can be linear or conformational.

Fab: The term “Fab” in the context of an engineered IgG of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab (a type of “domain exchanged” arrangement). Alternatively, or in addition to, the use of substituted or swapped constant domains, correct chain pairing can be achieved by the use of universal light chains that can pair with both variable regions of a heterodimeric binding molecule of the disclosure. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction and/or for purification, e.g., via star mutations.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Host Cell or Recombinant Host Cell: The terms “host cell” and “recombinant host cell” as used herein refer to a cell that has been genetically engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a multispecific binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Immune Response: The term “immune response” refers to an integrated bodily response to an antigen and preferably refers to a cellular immune response or a cellular as well as a humoral immune response. The immune response may be protective (also “preventive” or “prophylactic”) and/or therapeutic. The terms “inducing an immune response,” “eliciting an immune response,” and the like, as used herein, can indicate that there was no immune response against a particular antigen before administration of a particular composition (e.g., an MBM of the disclosure), but it may also indicate that there was a certain level of immune response against a particular antigen before such administration, and that after induction the immune response is enhanced. Thus, “inducing an immune response” also includes “enhancing an immune response”. Preferably, after inducing an immune response in a subject, said subject is protected from developing a disease (e.g., cancer) or the disease condition is ameliorated (e.g., tumor reduction, reduction in cancer cell number, etc.) by inducing an immune response. For example, an immune response against a tumor antigen may be induced in a patient having cancer or in a subject at risk of developing a cancer. Inducing an immune response in this case may mean that the disease condition of the subject is ameliorated, that the subject does not develop metastases, or that the subject at risk of developing cancer does not develop cancer.

Multivalent: The term “multivalent” as used herein refers to a binding molecule comprising two or more ABDs, on one, two or more polypeptide chains. A multivalent molecule may be monospecific, bispecific, or be specific for more than two epitopes, whether on the same antigen or different antigens.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence. In the context of a fusion protein or other polypeptide, the term “operably linked” means that two or more amino acid segments are linked so as to produce a functional polypeptide. In the context of a nucleic acid encoding a fusion protein, such as a multispecific binding molecule of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single chain Fv” or “scFv” as used herein refers to a polypeptide chain comprising the VH and VL domains of antibody, where these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315.

Single Domain Antibody or sdAb: The term “single domain antibody” or “sdAb” as used herein refers to an antibody or antigen binding fragment thereof comprising a single binding domain (e.g., heavy chain variable region) capable of binding a target molecule without pairing with a corresponding CDR-containing polypeptide (e.g., a light chain). An sdAb or sdAb fragment can be derived from a VHH or from a non-antibody scaffold protein, for example a designed ankyrin repeat protein (darpin), an avimer, an anticalin/lipocalin, a centyrin or a fynomer. A sdAb typically lacks a CH1 domain and thus cannot associate with a light chain.

Specifically (or Selectively) Binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In certain embodiments, the subject is human. Except when noted, the terms “patient” or “subject” are used herein interchangeably.

T Cell Antigen: The term “T cell antigen” (also “TCA”) as used herein refers to any biological molecule (e.g., protein, carbohydrate, lipid or combination thereof) that is present on and/or expressed by a T cell. In some embodiments, at least a portion of a T cell antigen is extracellular (e.g., is a cell surface protein or transmembrane protein having at least one extracellular domain). Particular T cell antigens contemplated herein include, but are not limited to, CD3 and CD28. In some embodiments, the T cell antigen is CD3.

Targeting Moiety: The term “targeting moiety” as used herein refers to any molecule or binding portion thereof that can specifically bind to an antigen. Exemplary targeting moieties include, but are not limited to, antibodies and antigen binding portions thereof (e.g., Fab, scFv, sdAb, etc.). A targeting moiety may be described with reference to the antigen to which it specifically binds. Thus, for example, a “T cell antigen targeting moiety” (or “TCA targeting moiety”) refers to a molecule or binding portion thereof that can specifically bind to a T cell antigen. The TCA targeting moiety can also have a functional activity in addition to binding a T cell antigen. For example, a TCA targeting moiety that is a CD3 targeting moiety (e.g., an anti-CD3 antibody or an antigen binding portion thereof) may facilitate clustering and activation of CD3 on a surface of a T cell, while a TCA targeting moiety that is a CD28 targeting moiety (e.g., an anti-CD28 antibody or an antigen binding portion thereof) may activate CD28 signaling in a T cell. Similarly a “TCR targeting moiety” refers to a molecule or binding portion thereof that can specifically bind to a T cell receptor.

Tetravalent: The term “tetravalent” as used herein refers to a binding molecule that has four antigen-binding domains. In certain embodiments, all four of the antigen-binding domains bind to the same epitope. In some embodiments, three of the antigen-binding domains bind to the same epitope and the other antigen-binding domain binds to a different epitope, whether of the same target molecule or different target molecules. In other embodiments, two of the antigen-binding domains bind to the same epitope and the other two antigen-binding domains bind to a different epitope, whether of the same target molecule or different target molecules. In further embodiments, all four of the antigen-binding domains bind to different epitopes, whether on the same target molecule or on any combination of two or more different target molecules. Accordingly, a tetravalent binding molecule may be monospecific, bispecific, trispecific, or tetraspecific.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” (also “tumor associated antigen”) or “TAA” refers to a molecule (typically a protein, carbohydrate, lipid or some combination thereof) that is expressed on the surface of a cancer cell, either entirely or as a fragment (e.g., as a peptide presented by an MHC molecule), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell.

In some embodiments, a TAA will be expressed exclusively on the cell surface of a cancer cell, entirely or as a fragment (e.g., as a peptide presented by an MHC molecule), and not synthesized or expressed on the surface of a normal cell. Accordingly, the term “TAA” encompasses antigens that are specific to cancer cells, sometimes known in the art as tumor-specific antigens (“TSAs”).

Universal Light Chain, ULC: The term “universal light chain” or “ULC” as used herein in the context of an antigen-biding domain refers to a light chain polypeptide capable of pairing with the heavy chain region of the antigen-biding domain and also capable of pairing with other heavy chain regions. Universal light chains are also known as “common light chains.”

VHH: The term “VHH” refers to a variable region of an antibody consisting of only a heavy chain, e.g., an antibody of camelid or cartilaginous fish origin. A VHH variable region can bind to a target molecule in the absence of a light chain. A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. Engineered IgG Molecules

The present disclosure provides engineered IgG molecules that include one or more IgM Cμ2 domains. The one or more IgM Cμ2 domains may fully or partially replace one or more IgG heavy chain hinge regions. Engineered IgG molecules described herein may be used in conjunction with various binding moieties including, for example, Fab moieties.

Incorporating IgM Cμ2 domains into an engineered IgG molecule may reduce the angle between Fab arms of an engineered IgG molecule as compared to a corresponding IgG molecule having native heavy chain constant regions. Incorporating IgM Cμ2 domains as described herein may also improve the activity of an engineered IgG molecule as compared to a corresponding IgG molecule having native heavy chain constant regions. Antibodies capable of stimulating signaling through a cell membrane receptor may become capable of stimulating signaling to a greater degree by replacing all or part of an IgG hinge region with an IgM Cμ2 domain.

### 6.2.1. IgM Cμ2 Domains

Embodiments of engineered IgG molecules of the present disclosure comprise Fc domains that include Cμ2 domains derived from IgM. IgM occurs naturally in humans as covalent multimers of heavy chain (H) light chain (L) assemblies forming a common H2L2 antibody unit. The IgM heavy chain constant region includes four constant domains, Cμ1, Cμ2, Cμ3, and Cμ4. In addition to the heavy and light chains, IgMs also possess a third chain, known as the joining (J)-chain (Keyt et al., 2020, Antibodies. 9(4):53). IgM occurs as a pentamer when it has incorporated a J chain, or as a hexamer when it lacks a J chain. IgM assembly typically starts with the association of a heavy (H) and a light (L) chain into a H-L arrangement, which then dimerizes to form H2L2 subunits. This intra-subunit assembly involves Cys337 of each of two Cμ2 domains, which forms a disulfide bond between the Cμ2 domains and stabilizes the H2L2. Next, these subunits are brought together by disulfide bridges to form multimers. A residue involved in this multimerization is Cys575 on tail domains of Cμ4, which forms disulfide bonds and enables noncovalent Cμ4 interactions. Another important residue is Cys414 on Cμ3, which further connects two Cμ3 domains of neighboring H2L2 subunits, in series to the disulfide bond between Cys337 residues of Cμ2.

Cμ2 domains for use in producing engineered IgG molecules of the present disclosure may include variants of the naturally occurring Cμ2 domains described above. In one example, the Fc region of the present disclosure comprises at least one constant domain that varies in sequence from the wildtype constant domain. It will be appreciated that the variant Cμ2 domains may be longer or shorter than their counterpart wild type Cμ2 domains. The following is the amino acid sequence of an exemplary wild type Cμ2 domain:

(SEQ ID NO: 1)
VIAELPPKVSVFVPPRDGFFGNPRKSKLICQATGFSPRQI
QVSWLREGKQVGSGVTTDQVQAEAKESGPTTYKVTSTLTI
KESDWLGQSMFTCRVDHRGLTFQQNASSMCVP

In some embodiments, engineered IgG molecules of the present disclosure comprise a sequence having at least 80%, 85%, 90%, 95%, or 99% sequence identity to the amino acid sequence of SEQ ID NO: 1. In some embodiments, engineered IgG molecules of the present disclosure comprise the amino acid sequence of SEQ ID NO: 1.

In some embodiments, engineered IgG molecules do not include a Cμ1 domain, or a sequence derived from a Cμ1 domain. An exemplary amino acid sequence of a Cμ1 domain is as follows:

(SEQ ID NO: 2)
APTLFPLVSCENSPSDTSSVAVGCLAQDFLPDSITLSWKYK
NNSDISSTRGFPSVLRGGKYAATSQVLLPSKDVMQGTDEHV
VCKVQHPNGNKEKNVP

In some embodiments, the engineered IgG molecule lacks an amino acid sequence having at least 90% sequence identity to the amino acid sequence of SEQ ID NO: 2. In some embodiments, the engineered IgG molecule lacks an amino acid sequence having at least 90% sequence identity to 10, 15, 20, or 30 or more continuous amino acids of SEQ ID NO: 2.

### 6.2.2. IgG Constant Domains

Embodiments of engineered IgG molecules disclosed herein may include IgG constant domains, such as, for example, IgG Fc domains. In native antibodies, the heavy chain Fc domain of IgG is composed of two heavy chain constant domains (CH2 and CH3).

These dimerize to create an Fc region. The Fc domains that can be incorporated into engineered IgG molecules of the present disclosure can be derived from any IgG subclass (i.e., IgG1, IgG2, IgG3 and IgG4). In some embodiments, one or both pairs of Fc domains are derived from IgG1. In some embodiments, one or both pairs of Fc domains are derived from IgG2, IgG3, or IgG4. In some embodiments, the Fc region comprises CH2 and CH3 domains derived from IgG1. In some embodiments, the Fc region comprises CH2 and CH3 domains derived from IgG2. In some embodiments, the Fc region comprises CH2 and CH3 domains derived from IgG3. In some embodiments, the Fc region comprises CH2 and CH3 domains derived from IgG4.

The two Fc domains within the Fc region of an engineered IgG molecule can be the same or different from one another. In a native antibody the Fc domains are typically identical, but for the purpose of producing molecules with different binding domains or other asymmetries, the Fc domains might advantageously be different to allow for heterodimerization, as described in Section 6.2.2.2 below.

Heavy chain constant domains used in engineered IgG molecules of the disclosure may have IgG hinge regions that are partially or completely removed. Native IgG molecules have hinge domains between CH1 and CH2 domains. Exemplary hinge domain sequences are set forth in Table A below.

TABLE A
IgG Hinge Domain Sequences

| | | SEQ ID |
|---|---|---|
| Hinge Domain | Sequence | NO |
| hIgG1 Hinge | EPKSCDKTHTCPPCP | 3 |
| hIgG2 Hinge | ERKCCVECPPCP | 4 |
| hIgG4 Hinge | ESKYGPPCPSCP | 5 |

In some embodiments, engineered IgG molecules of the disclosure lack an intact IgG hinge domain. In some embodiments, the engineered IgG molecules lack an intact IgG hinge domain between CH1 and CH2 domains or between CH1 and IgM Cμ2 domains. In some embodiments, the engineered IgG molecules have an IgG hinge domain of reduced length relative to a wild type IgG hinge domain. In some embodiments, the engineered IgG molecules do not have more than 1, 2, 3, 4, or 5 amino acids derived from an IgG hinge domain between CH1 and CH2 domains or between CH1 and IgM Cμ2 domains. In some embodiments, the engineered IgG molecules do not include a sequence that has greater than 80%, 85%, or 90% sequence identity to the amino acid sequence of SEQ ID NO:3. In some embodiments, the engineered IgG molecules do not include a sequence that has greater than 80%, 85%, or 90% sequence identity to the amino acid sequence of SEQ ID NO:3 between CH1 and CH2 domains or between CH1 and IgM Cμ2 domains. In some embodiments, the engineered IgG molecules do not include a sequence that has greater than 80%, 85%, or 90% sequence identity to the amino acid sequence of SEQ ID NO:4. In some embodiments, the engineered IgG molecules do not include a sequence that has greater than 80%, 85%, or 90% sequence identity to the amino acid sequence of SEQ ID NO:4 between CH1 and CH2 domains or between CH1 and IgM Cμ2 domains. In some embodiments, the engineered IgG molecules do not include a sequence that has greater than 80%, 85%, or 90% sequence identity to the amino acid sequence of SEQ ID NO:5. In some embodiments, the engineered IgG molecules do not include a sequence that has greater than 80%, 85%, or 90% sequence identity to the amino acid sequence of SEQ ID NO:5 between CH1 and CH2 domains or between CH1 and IgM Cμ2 domains.

Engineered IgG molecules of the present disclosure may include one or more IgG CH1 domains. The CH1 domain may be an IgG1 CH1 domain, an IgG2 CH2 domain, an IgG3 CH1 domain, or an IgG4 CH1 domain. Such CH1 domains may be present on an engineered IgG heavy chain polypeptide or on an engineered IgG light chain polypeptide.

Engineered IgG molecules of the present disclosure may include one or more CL domains. The CL domains may be, for example, a K light chain constant domain or a λ light chain constant domain. Such CL domains may be present on an engineered IgG heavy chain polypeptide or an engineered IgG light chain polypeptide.

Fc domains, such as IgG CH2 or CH3 domains, may have additional modifications such as, for example, deletion of the amino acids AP at the N-terminus of the IgG CH2 domain generally present in the wildtype sequence. In some embodiments, the amino acids PLP are added to the N-terminus of the IgM Cμ2 domain. In some embodiments, these modifications are combined, such that the IgM Cμ2 domain has the amino acids PLP added to its N-terminus and the IgG CH2 domain has the amino acids AP at the N-terminus deleted.

It will be appreciated that the heavy chain constant domains for use in producing an Fc region for the engineered IgG molecules of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

The Fc domains that are incorporated into the engineered IgG molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.2.2.1.

The Fc domains can also be altered to include modifications that improve manufacturability of engineered IgG molecules, for example by allowing heterodimerization, which is the preferential pairing of non-identical Fc domains over identical Fc domains.

Heterodimerization permits the production of multivalent engineered IgG molecules in which different polypeptide components are connected to one another by an Fc region containing Fc domains that differ in sequence. Examples of heterodimerization strategies are exemplified in Section 6.2.2.1.

It will be appreciated that any of the modifications mentioned above can be combined in any suitable manner to achieve the desired functional properties and/or combined with other modifications to alter the properties of the engineered IgG molecules.

Example Fc domain sequences are provided in Table F-1, below. In some embodiments, an Fc domain comprises an amino acid sequence having at least 90%, at least 95%, at least 98%, at least 99%, or 100% sequence identity with an amino acid sequence set forth in Table F-1. In some embodiments, the exemplary Fc domain sequences provided in Table F-1 below are modified by having their hinge sequences partially or completely replaced by an IgM Cμ2 domain sequence, as described herein.

TABLE F-1
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVTCVVVDVS | 6 |
| (amino acids 99- | HEDPEVKFNWYVDGVEVHNAKTKPREEQYNSTYRVVSVLTVLHQDWLNGKEY | |
| 330 of UniprotKB | KCKVSNKALPAPIEKTISKAKGQPREPQVYTLPPSRDELTKNQVSLTCLVKG | |
| P01857-1) | FYPSDIAVEWESNGQPENNYKTTPPVLDSDGSFFLYSKLTVDKSRWQQGNVF | |
| | SCSVMHEALHNHYTQKSLSLSPGK | |
| hIgG2 Fc | ERKCCVECPPCPAPPVAGPSVFLFPPKPKDTLMISRTPEVTCVVVDVSHEDP | 7 |
| (amino acids 99- | EVQFNWYVDGVEVHNAKTKPREEQFNSTFRVVSVLTVVHQDWLNGKEYKCKV | |
| 326 of UniprotKB | SNKGLPAPIEKTISKTKGQPREPQVYTLPPSREEMTKNQVSLTCLVKGFYPS | |
| P01859-1) | DISVEWESNGQPENNYKTTPPMLDSDGSFFLYSKLTVDKSRWQQGNVFSCSV | |
| | MHEALHNHYTQKSLSLSPGK | |
| hIgG3 Fc | ELKTPLGDTTHTCPRCPEPKSCDTPPPCPRCPEPKSCDTPPPCPRCPEPKSC | 8 |
| (amino acids 99- | DTPPPCPRCPAPELLGGPSVFLFPPKPKDTLMISRTPEVTCVVVDVSHEDPE | |
| 377 of UniprotKB | VQFKWYVDGVEVHNAKTKPREEQYNSTFRVVSVLTVLHQDWLNGKEYKCKVS | |
| P01860-1) | NKALPAPIEKTISKTKGQPREPQVYTLPPSREEMTKNQVSLTCLVKGFYPSD | |
| | IAVEWESSGQPENNYNTTPPMLDSDGSFFLYSKLTVDKSRWQQGNIFSCSVM | |
| | HEALHNRFTQKSLSLSPGK | |
| hIgG4 Fc | ESKYGPPCPSCPAPEFLGGPSVFLFPPKPKDTLMISRTPEVTCVVVDVSQED | 9 |
| (amino acids 99- | PEVQFNWYVDGVEVHNAKTKPREEQFNSTYRVVSVLTVLHQDWLNGKEYKCK | |
| 327 of UniprotKB | VSNKGLPSSIEKTISKAKGQPREPQVYTLPPSQEEMTKNQVSLTCLVKGFYP | |
| P01861-1) | SDIAVEWESNGQPENNYKTTPPVLDSDGSFFLYSRLTVDKSRWQEGNVFSCS | |
| | VMHEALHNHYTQKSLSLSLGK | |
| hIgG4s Fc | ESKYGPPCPPCPAPPVAGPSVFLFPPKPKDTLMISRTPEVTCVVVDVSQEDP | 10 |
| | EVQFNWYVDGVEVHNAKTKPREEQFNSTYRVVSVLTVLHQDWLNGKEYKCKV | |
| | SNKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCSV | |
| | MHEALHNHYTQKSLSLSLGK | |
| hIgG1 PVA Fc | EPKSCDKTHTCPPCPAPPVAGPSVFLFPPKPKDTLMISRTPEVTCVVVDVSH | 11 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 PVA/P329A | EPKSCDKTHTCPPCPAPPVAGPSVFLFPPKPKDTLMISRTPEVTCVVVDVSH | 12 |
| Fc | EDPEVKFNWYVDGVEVHNAKTKPREEQYNSTYRVVSVLTVLHQDWLNGKEYK | |
| | CKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 LALAPG Fc | EPKSCDKTHTCPPCPAPEAAGGPSVFLFPPKPKDTLMISRTPEVTCVVVDVS | 13 |
| | HEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG1 PVA star | EPKSCDKTHTCPPCPAPPVAGPSVFLFPPKPKDTLMISRTPEVTCVVVDVSH | 14 |
| | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| hIgG1s | DKKVEPKSCDKTHTCPPCPAPPVAGPSVFLFPPKPKDTLMISRTPEVTCVVV | 15 |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| hIgG1 N180G, | EPKSCDKTHTCPPCPAPELLGGPSVFLFPPKPKDTLMISRTPEVTCVVVDVS | 16 |
| also referred | HEDPEVKFNWYVDGVEVHNAKTKPREEQYGSTYRVVSVLTVLHQDWLNGKEY | |
| to as | KCKVSNKALPAPIEKTISKAKGQPREPQVYTLPPSRDELTKNQVSLTCLVKG | |
| N297G | FYPSDIAVEWESNGQPENNYKTTPPVLDSDGSFFLYSKLTVDKSRWQQGNVF | |
| | SCSVMHEALHNHYTQKSLSLSPGK | |
| hIgG2 variant | ERKCCVECPPCPAPPVAGPSVFLFPPKPKDTLMISRTPEVTCVVVDVSHEDP | 17 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DIAVEWESNGQPENNYKTTPPMLDSDGSFFLYSKLTVDKSRWQQGNVFSCSV | |
| | MHEALHNHYTQKSLSLSPGK | |
| hIgG4 S108P | ESKYGPPCPPCPAPEFLGGPSVFLFPPKPKDTLMISRTPEVTCVVVDVSQED | 18 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLGK | |
| IgG1PVA_hinge- | EPKSCDKTHTCPPCPAPPVAGPSVFLFPPKPKDTLMISRTPEVTCVVVDVSH | 19 |
| Fc knob | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 20 |
| Fc knob_star | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLWCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 21 |
| Fc knob_Cys | EDPEVKFNWYVDGVEVHNAKTKPREEQYNSTYRVVSVLTVLHQDWLNGKEYK | |
| | CKVSNKALPAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 22 |
| Fc knob_ | EDPEVKFNWYVDGVEVHNAKTKPREEQYNSTYRVVSVLTVLHQDWLNGKEYK | |
| Cys_star | CKVSNKALPAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 23 |
| Fc Hole | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 24 |
| Fc Hole_star | EDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNRFTQKSLSLSPGK | |
| IgG1PVA_hinge- | EPKSCDKTHTCPPCPAPPVAGPSVFLFPPKPKDTLMISRTPEVTCVVVDVSH | 25 |
| Fc Hole_Cys | EDPEVKFNWYVDGVEVHNAKTKPREEQYNSTYRVVSVLTVLHQDWLNGKEYK | |
| | CKVSNKALPAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNHYTQKSLSLSPGK | |
| IgG1PVA_hinge- | EPKSCDKTHTCPPCPAPPVAGPSVFLFPPKPKDTLMISRTPEVTCVVVDVSH | 26 |
| Fc Hole_Cys_ | EDPEVKFNWYVDGVEVHNAKTKPREEQYNSTYRVVSVLTVLHQDWLNGKEYK | |
| star | CKVSNKALPAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQGNVFS | |
| | CSVMHEALHNRFTQKSLSLSPGK | |

### 6.2.2.1. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain or the Fc region (e.g., one or both Fc domains of an engineered IgG molecule that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In some embodiments, the Fc domain has one or more modifications that increase binding to FcγRIIB. In some embodiments, one or both Fc regions have S239D and 1332E amino acid substitutions (Kabat EU index numbering). In some embodiments, one or both Fc regions have S239D, A330L, and 1332E amino acid substitutions (Kabat EU index numbering). In some embodiments, one or both Fc regions have G236A amino acid substitutions (Kabat EU index numbering). In some embodiments, one or both Fc regions have G236A, S239D, and 1332E amino acid substitutions (Kabat EU index numbering). In some embodiments, one or both Fc regions have G236A, S239D, A330L, and 1332E amino acid substitutions (Kabat EU index numbering). In some embodiments, one or both Fc regions have F243L, R292P, Y300L, V3051, and P396L amino acid substitutions (Kabat EU index numbering). In some embodiments, one or both Fc regions have S239D and 1332E amino acid substitutions (Kabat EU index numbering).

In another embodiment, the Fc domain is an IgG4 Fc domain with reduced binding to Fc receptors. Exemplary IgG4 Fc domains with reduced binding to Fc receptors may comprise an amino acid sequence selected from Table C below. In some embodiments, the exemplary Fc domain sequences provided in Table C are modified by having their hinge sequences partially or completely replaced by an IgM Cμ2 domain sequence, as described herein. In some embodiments, the Fc domain includes only the bolded portion of the sequences shown below:

TABLE C

| | | SEQ |
|---|---|---|
| Fc Domain | Sequence | ID NO |
| SEQ ID NO: 1 of | DKRVESKYGP PCPPCPAPPV AGPSVFLFPP KPKDTLMISR | 27 |
| WO2014/121087 | TPEVTCVVVD VSQEDPEVQF NWYVDGVEVH NAKTKPREEQ | |
| | FNSTYRVVSV LTVLHQDWLN GKEYKCKVSN KGLPSSIEKT | |
| | ISKAKGQPRE PQVYTLPPSQ EEMTKNQVSL TCLVKGFYPS | |
| | DIAVEWESNG QPENNYKTTP PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL FPPKPKDTLM | 28 |
| WO2014/121087 | ISRTPEVTCV VVDVSQEDPE VQFNWYVDGV EVHNAKTKPR | |
| | EEQFNSTYRV VSVLTVLHQD WLNGKEYKCK VSNKGLPSSI | |
| | EKTISKAKGQ PREPQVYTLP PSRDELTKNQ VSLTCLVKGF | |
| | YPSDIAVEWE SNGQPENNYK TTPPVLDSDG SFFLYSKLTV | |
| | DKSRWQQGNV FSCSVMHEAL HNHYTQKSLS LSPGK | |
| SEQ ID NO: 30 | ASTKGPSVFP LAPSSKSTSG GTAALGCLVK DYFPEPVTVS | 29 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPGK | |
| SEQ ID NO: 31 | ASTKGPSVFP LAPCSRSTSE STAALGCLVK DYFPEPVTVS | 30 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP REEQFNSTYR VVSVLTVLHQ DWLNGKEYKC | |
| | KVSNKGLPSS IEKTISKAKG QPREPQVYTL PPSQEEMTKN | |
| | QVSLTCLVKG FYPSDIAVEW ESNGQPENNY KTTPPVLDSD | |
| | GSFFLYSRLT VDKSRWQEGN VFSCSVMHEA LHNHYTQKSL | |
| | SLSLGK | |
| SEQ ID NO: 37 | ASTKGPSVFP LAPSSKSTSG GTAALGCLVK DYFPEPVTVS | 31 |
| of | WNSGALTSGV HTFPAVLQSS GLYSLSSVVT VPSSSLGTQT | |
| WO2014/121087 | YICNVNHKPS NTKVDKKVEP KSCDKTHTCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNRFTQ | |
| | KSLSLSPGK | |
| SEQ ID NO: 38 | ASTKGPSVFP LAPCSRSTSE STAALGCLVK DYFPEPVTVS | 32 |
| of | WNSGALTSGV HTFPAVLQSS GLYSLSSVVT VPSSSLGTKT | |
| WO2014/121087 | YTCNVDHKPS NTKVDKRVES KYGPPCPPCP APPVAGPSVF | |
| | LFPPKPKDTL MISRTPEVTC VVVDVSQEDP EVQFNWYVDG | |
| | VEVHNAKTKP IEKTISKAKG QPREPQVYTL PPSQEEMTKN | |
| | KVSNKGLPSS FYPSDIAVEW ESNGQPENNY KTTPPVLDSD | |
| | QVSLTCLVKG VDKSRWQEGN VFSCSVMHEA LHNRFTQKSL | |
| | GSFFLYSRLT REEQFNSTYR VVSVLTVLHQ DWLNGKEYKC | |
| | SLSLGK | |

In a particular embodiment, the IgG4 with reduced effector function comprises the bolded portion of the amino acid sequence of SEQ ID NO:30 (SEQ ID NO:31 of WO2014/121087), sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:29 (SEQ ID NO:30 of WO2014/121087) (or the bolded portion thereof) and an Fc domain comprising the amino acid sequence of SEQ ID NO:31 (SEQ ID NO:37 of WO2014/121087) (or the bolded portion thereof) or an Fc region comprising an Fc domain comprising the amino acid sequence of SEQ ID NO:30 (SEQ ID NO:31 of WO2014/121087) (or the bolded portion thereof) and an Fc domain comprising the amino acid sequence of SEQ ID NO:32 (SEQ ID NO:38 of WO2014/121087) (or the bolded portion thereof).

### 6.2.2.2. Fc Heterodimerization Variants

Certain engineered IgG molecules entail dimerization between two Fc domains that, unlike a native immunoglobulin, are operably linked to non-identical N-terminal regions, e.g., one Fc domain connected to a first targeting moiety and the other Fc domain connected to a second, different, targeting moiety. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the engineered IgG molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides engineered molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any IgG subclass, as described above.

Heterodimerization of the two different heavy chains at CH3 domains give rise to the desired engineered IgG molecule, while homodimerization of identical heavy chains will reduce yield of the desired engineered IgG molecule. Thus, in some embodiments, the polypeptides that associate to form an engineered IgG molecule of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, engineered IgG molecules may comprise a first CH3 domain and a second Ig CH3 domain, wherein the first and second Ig CH3 domains differ from one another by at least one amino acid, and wherein at least one amino acid difference reduces binding of the engineered IgG to Protein A as compared to a corresponding engineered IgG lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.2.3. Targeting Moieties

Engineered IgG molecules described herein may include one or more binding moieties. The one or more binding moieties may include, for example, one or more Fab domains. The one or more Fab domains may be, for example, in a native arrangement (i.e., with a CH1 domain linked to a VH domain and a CL domain linked to a VL domain) or in a Crossmab arrangement (i.e., with a CH1 domain linked to a VL domain and a CL domain linked to a VH domain). It will be recognized that other types of targeting moieties may also be included in the engineered IgG molecules of the present disclosure.

Targeting moieties included in the engineered IgG molecules of the present disclosure may specifically bind to one or more target molecules. Target molecules may include, for example, cell surface receptors that can be agonized by an antibody having targeting moieties that bind to the target molecule. For example, the one or more target molecules may be CD40, OX40, GITR, 4-1BB, CD27, HVEM, CD30, leptin receptor (LEPR), insulin-like growth factor 1 receptor (IGF1R), erythropoietin (EPO) receptor, G-CSF receptor, leptin receptor (LEPR), or prolactin receptor (PRLR).

In some embodiments, one or more targeting moieties of an engineered IgG molecule of the present disclosure selectively binds a member of the TNF receptor superfamily, also referred to herein as TNF family receptors. In some embodiments, the TNF family receptor is CD40, OX40, glucocorticoid-induced TNFR-related protein (GITR), 4-1BB, CD27, herpesvirus entry mediator (HVEM), or CD30. Activation of a TNF family receptor may be useful in methods of activating immune cells and treating diseases such as cancer.

In some embodiments, one or more targeting moieties of an engineered IgG molecule of the present disclosure selectively binds a homodimeric class I cytokine receptor.

In some embodiments, the homodimeric class I cytokine receptor is EPO receptor, G-CSF receptor, leptin receptor (LEPR), or prolactin receptor PRLR. Embodiments of engineered IgG molecules that bind to an EPO receptor target molecule may include targeting moieties that bind one or more epitopes of the EpoR protein. Embodiments of engineered IgG molecules that bind to a G-CSF receptor target molecule (a homodimer receptor) may include targeting moieties that bind one or more epitopes of the G-CSFR protein.

Embodiments of engineered IgG molecules that bind to a leptin receptor target molecule (a homodimer receptor) may include targeting moieties that bind one or more epitopes of the LEPR protein. Embodiments of engineered IgG molecules that bind to a prolactin receptor target molecule (a homodimer receptor) may include targeting moieties that bind one or more epitopes of the PRLR protein.

Exemplary targeting moieties that may be used in embodiments of engineered IgG molecules include the targeting moieties from the antibodies set forth in Table B below.

TABLE B
Exemplary Targeting Moieties

| Target | Reference | Sequence |
|---|---|---|
| LEPR | VH: SEQ ID NO: 2 of | VH: |
| | U.S. Publication No. | QVQLVESGGGVVQPGKSLRLSCVASGFTFSSDAMYWVRQAPGKGL |
| | US 2019/0309079 | EWVAVIYYDGNYQYYEDSVKGRFTISRDNSQNTLDLQMNSLRVDD |
| | A1; | TAVYFCARLNWDYWYLDLWGRGTLVTVSS (SEQ ID NO: 40) |
| | VL: SEQ ID NO: 10 | VL: |
| | of U.S. Publication | DIQMTQSPSSLSASVGDRVTITCRASQSISSYLNWYQQKPGKAPK |
| | No. US | LLIYAASSLQSGVPSRFSGSGSGTDFTLTISSLQPEDFATYYCQQ |
| | 2019/0309079 A1 | SYSTPPITFGQGTRLEIK (SEQ ID NO: 41) |
| LEPR | VH: SEQ ID NO: 18 | VH: |
| | of U.S. Publication | QVQLVESGGGVVQPGRSLRLSCTASGFTFSSYAMYWVRQAPGKGL |
| | No. US | EWVSVIYYDGSYKYYADSVKGRFTISRDNSKNTLYLQMDSLRAED |
| | 2019/0309079 A1; | TAVYYCASYNWNYWYFDFWGRGTLVTVSS (SEQ ID NO: 42) |
| | VL: SEQ ID NO: 10 | VL: |
| | of U.S. Publication | DIQMTQSPSSLSASVGDRVTITCRASQSISSYLNWYQQKPGKAPK |
| | No. US | LLIYAASSLQSGVPSRFSGSGSGTDFTLTISSLQPEDFATYYCQQ |
| | 2019/0309079 A1 | SYSTPPITFGQGTRLEIK (SEQ ID NO: 41) |
| LEPR | VH: SEQ ID NO: 26 | VH: |
| | of U.S. Publication | QVQLVESGGSVVQPGRSLRLSCAASGFTFSTYAMYWVRQTPGKGL |
| | No. US | EWVAVLYSDGSNKYYIDSVKGRFTISRDTSTNTLYLQMSSLRADD |
| | 2019/0309079 A1; | SALYYCARLNWDYWYFDLWGRGTLVTVSS (SEQ ID NO: 43) |
| | VL: SEQ ID NO: 10 | VL: |
| | of U.S. Publication | DIQMTQSPSSLSASVGDRVTITCRASQSISSYLNWYQQKPGKAPK |
| | No. US | LLIYAASSLQSGVPSRFSGSGSGTDFTLTISSLQPEDFATYYCQQ |
| | 2019/0309079 A1 | SYSTPPITFGQGTRLEIK (SEQ ID NO: 41) |
| LEPR | VH: SEQ ID NO: 34 | VH: |
| | of U.S. Publication | QVQLVESGGGVVQPGRSLRLSCEASGFSSSDNAMYWVRQAPGKGL |
| | No. US | EWVSVIYHDGSYKYYEDSVKGRFTIARDNSKNTLYLQMNSLRAED |
| | 2019/0309079 A1; | TAVYYCARYNWNHWYFDVWGRGTLVTVSS (SEQ ID NO: 44) |
| | | VL: |
| | VL: SEQ ID NO: 10 | DIQMTQSPSSLSASVGDRVTITCRASQSISSYLNWYQQKPGKAPK |
| | of U.S. Publication | LLIYAASSLQSGVPSRFSGSGSGTDFTLTISSLQPEDFATYYCQQ |
| | No. US | SYSTPPITFGQGTRLEIK (SEQ ID NO: 41) |
| | 2019/0309079 A1 | |
| LEPR | VH: SEQ ID NO: 42 | VH: |
| | of U.S. Publication | QVQLVESGGGVVQPGRSLRLSCAASGFTFSTYGMHWVRQAPGKGL |
| | No. US | EWVSVISYDESNKYYADSVKGRFTISRDNSKNALYLQMNSLRNED |
| | 2019/0309079 A1; | TAVYYCARDRPFGLVTGWFDPWGQGTLVTVSS (SEQ ID NO: |
| | VL: SEQ ID NO: 10 | 45) |
| | of U.S. Publication | VL: |
| | No. US | DIQMTQSPSSLSASVGDRVTITCRASQSISSYLNWYQQKPGKAPK |
| | 2019/0309079 A1 | LLIYAASSLQSGVPSRFSGSGSGTDFTLTISSLQPEDFATYYCQQ |
| | | SYSTPPITFGQGTRLEIK (SEQ ID NO: 41) |
| LEPR | VH: SEQ ID NO: 50 | VH: |
| | of U.S. Publication | QVQLVESGGGVVQPGRSLRLSCAASGFSFNTYGMHWVRQAPGKGL |
| | No. US | EWVTIIWYDGSIKYYADSVKGRFTISRDNSKNTLYLQMNSLRAED |
| | 2019/0309079 A1; | TAVYYCARGGYSGYLYFDYWGQGTLVTVSS (SEQ ID NO: |
| | VL: SEQ ID NO: 10 | 46) |
| | of U.S. Publication | VL: |
| | No. US | DIQMTQSPSSLSASVGDRVTITCRASQSISSYLNWYQQKPGKAPK |
| | 2019/0309079 A1 | LLIYAASSLQSGVPSRFSGSGSGTDFTLTISSLQPEDFATYYCQQ |
| | | SYSTPPITFGQGTRLEIK (SEQ ID NO: 41) |
| LEPR | VH: SEQ ID NO: 58 | VH: |
| | of U.S. Publication | QVQLQESGPGLVKPSQTLSLTCTVSGGSISSGGDYWSWIRQLPGK |
| | No. US | GLEWIGYIYYSGSAYYNPSLKSRGTISIDTSKNQFSLKLTSVTAA |
| | 2019/0309079 A1; | DTAVYFCVKLRFLEWFLGGWFGPWGQGTLVTVSS (SEQ ID |
| | VL: SEQ ID NO: 10 | NO: 47) |
| | of U.S. Publication | VL: |
| | No. US | DIQMTQSPSSLSASVGDRVTITCRASQSISSYLNWYQQKPGKAPK |
| | 2019/0309079 A1 | LLIYAASSLQSGVPSRFSGSGSGTDFTLTISSLQPEDFATYYCQQ |
| | | SYSTPPITFGQGTRLEIK (SEQ ID NO: 41) |
| LEPR | VH: SEQ ID NO: 66 | VH: |
| | of U.S. Publication | QVQLQESGPGLVKPSQTLSLTCTVSGGSISSGGDYWSWIRQLPGK |
| | No. US | GLEWIGYIYYSGSAYYNPSLKSRGTISIDTSKNQFSLKLTSVTAA |
| | 2019/0309079 A1; | DTAVYFCVKLRFLEWFLGGWFGPWGQGTLVTVSS (SEQ ID |
| | VL: SEQ ID NO: 10 | NO: 47) |
| | of U.S. Publication | VL: |
| | No. US | DIQMTQSPSSLSASVGDRVTITCRASQSISSYLNWYQQKPGKAPK |
| | 2019/0309079 A1 | LLIYAASSLQSGVPSRFSGSGSGTDFTLTISSLQPEDFATYYCQQ |
| | | SYSTPPITFGQGTRLEIK (SEQ ID NO: 41) |
| LEPR | VH: SEQ ID NO: 74 | VH: |
| | of U.S. Publication | EVQLVESGGGLVQPGGSLRLSCVASGFTFNKYDMHWVRQTTGKGL |
| | No. US | EWVSGIDTDGDTYYPGSVKGRFTISRENAENSLYLQMNGLRVGDT |
| | 2019/0309079 A1; | AVYYCARWPWSGFYGAFDIWGQGTMVTVSS (SEQ ID NO: |
| | VL: SEQ ID NO: 10 | 48) |
| | of U.S. Publication | VL: |
| | No. US | DIQMTQSPSSLSASVGDRVTITCRASQSISSYLNWYQQKPGKAPK |
| | 2019/0309079 A1 | LLIYAASSLQSGVPSRFSGSGSGTDFTLTISSLQPEDFATYYCQQ |
| | | SYSTPPITFGQGTRLEIK (SEQ ID NO: 41) |
| LEPR | VH: SEQ ID NO: 82 | VH: |
| | of U.S. Publication | QVQLQESGPGLVKPSQTLSLTCTVSGGSISSGNYYWNWIRQQPGE |
| | No. US | GLEWIAYIYHNGVTNFNPSLKSRLTISVDTSKTQFSLKLRSVTAA |
| | 2019/0309079 A1; | DTAVYYCARSGSWFENWYFDLWGRGTLVTVSS (SEQ ID NO: |
| | VL: SEQ ID NO: 90 | 49) |
| | of U.S. Publication | VL: |
| | No. US | EIVLTQSPGTLSLSPGERATLSCRASQSVSSSYLAWYQQKPGQAP |
| | 2019/0309079 A1 | RLLIYGASSRATGIPDRFSGSGSGTDFTLTISRLEPEDFAVYYCQ |
| | | QYGSSPWTFGQGTKVEIK (SEQ ID NO: 50) |
| LEPR | VH: SEQ ID NO: 98 | VH: |
| | of U.S. Publication | QVQLQESGPGLVKPSETLSLTCTVSGGSISNSYWSWIRQPPGKGL |
| | No. US | EWIGYVYSRGNTKYNPSLTSRVTMSFDTSKNQFSLKLRSVTAADT |
| | 2019/0309079 A1; | AVYYCARSSSWYEDWYFDLWGRGTLVTVSS (SEQ ID NO: |
| | VL: SEQ ID NO: 90 | 51) |
| | of U.S. Publication | VL: |
| | No. US | EIVLTQSPGTLSLSPGERATLSCRASQSVSSSYLAWYQQKPGQAP |
| | 2019/0309079 A1 | RLLIYGASSRATGIPDRFSGSGSGTDFTLTISRLEPEDFAVYYCQ |
| | | QYGSSPWTFGQGTKVEIK (SEQ ID NO: 50) |
| LEPR | VH: SEQ ID NO: 106 | VH: |
| | of U.S. Publication | QVQLQQWGAGLFKPSETLSLTCDVYGGSFRGYYWSWIRQPPGKGL |
| | No. US | EWIGEISYSGFTNYNPSLKSRVIISIDTSKNQFSLKMSSVTAADT |
| | 2019/0309079 A1; | AVYYCARVTYGYGTFDYWGQGTLVTVSS (SEQ ID NO: 52) |
| | VL: SEQ ID NO: 90 | VL: |
| | of U.S. Publication | EIVLTQSPGTLSLSPGERATLSCRASQSVSSSYLAWYQQKPGQAP |
| | No. US | RLLIYGASSRATGIPDRFSGSGSGTDFTLTISRLEPEDFAVYYCQ |
| | 2019/0309079 A1 | QYGSSPWTFGQGTKVEIK (SEQ ID NO: 50) |
| CD40 | huAb8-1 VH: SEQ | VH: |
| | ID NO: 113 of PCT | EVQLVQSGAEVKKPGSSVKVSCKASGYTFTDYYINWVRQAPGQGL |
| | Application NO: WO | EWIGWIFPGSGSVYCNEQFKGRATLTVDRSTSTAYMELSSLRSED |
| | 2017/205742 A1; | TAVYFCASSLGKFAYWGQGTLVTVSS (SEQ ID NO: 53) |
| | VL: SEQ ID NO: 162 | VL: |
| | of PCT Application | DIQMTQSPSSLSASVGDRVTITCKASQSVVTAVAWYQQKPGKSPK |
| | NO: WO | LLIYSASNRYTGVPSRFSGSGSGTDFTLTISSLQPEDFATYFCQQ |
| | 2017/205742 A1 | YSSYPYTFGGGTKVEIK (SEQ ID NO: 54) |
| CD40 | huAb8-2 VH: SEQ | VH: |
| | ID NO: 114 of PCT | EVQLVQSGAEVKKPGSSVKVSCKASGYTFTDYYINWVRQAPGQGL |
| | Application NO: WO | EWIGWIFPGSGSVYSNEQFKGRATLTVDRSTSTAYMELSSLRSED |
| | 2017/205742 A1; | TAVYFCASSLGKFAYWGQGTLVTVSS (SEQ ID NO: 55) |
| | VL: SEQ ID NO: 162 | VL: |
| | of PCT Application | DIQMTQSPSSLSASVGDRVTITCKASQSVVTAVAWYQQKPGKSPK |
| | NO: WO | LLIYSASNRYTGVPSRFSGSGSGTDFTLTISSLQPEDFATYFCQQ |
| | 2017/205742 A1 | YSSYPYTFGGGTKVEIK (SEQ ID NO: 54) |
| CD40 | huAb8-3 VH: SEQ | VH: |
| | ID NO: 115 of PCT | EVQLVQSGAEVKKPGSSVKVSCKASGYTFTDYYINWVRQAPGQGL |
| | Application NO: WO | EWIGWIFPGSGSVYCNEQFKGRVTITVDKSTSTAYMELSSLRSED |
| | 2017/205742 A1; | TAVYYCASSLGKFAYWGQGTLVTVSS (SEQ ID NO: 56) |
| | VL: SEQ ID NO: 162 | VL: |
| | of PCT Application | DIQMTQSPSSLSASVGDRVTITCKASQSVVTAVAWYQQKPGKSPK |
| | NO: WO | LLIYSASNRYTGVPSRFSGSGSGTDFTLTISSLQPEDFATYFCQQ |
| | 2017/205742 A1 | YSSYPYTFGGGTKVEIK (SEQ ID NO: 54) |
| CD40 | huAb9-1 VH: SEQ | VH: |
| | ID NO: 116 of PCT | EVQLQESGPGLVKPSETLSLTCTVSGYSITSNYYWNWIRQPPGKG |
| | Application NO: WO | LEWMGYIRYDGSNNYNPSLKNRITISRDTSKNQFSLKLSSVTAAD |
| | 2017/205742 A1; | TAVYYCARLDYWGQGTTVTVSS (SEQ ID NO: 57) |
| | VL: SEQ ID NO: 163 | VL |
| | of PCT Application | DAVMTQTPLSLSVTPGQPASISCRSSQSLENTNGNTFLNWFLQKP |
| | NO: WO | GQSPQLLIYRVSNRFSGVPDRFSGSGSGTDFTLKISRVEAEDVGV |
| | 2017/205742 A1 | YFCLQVTHVPFTFGQGTKLEIK (SEQ ID NO: 58) |
| CD40 | huAb9-2 VH: SEQ | VH: |
| | ID NO: 117 of PCT | EVQLQESGPGLVKPSETLSLTCTVSGYSITSNYYWNWIRQPPGKG |
| | Application NO: WO | LEWMGYIRYDGSNNYNPSLKNRVTISRDTSKNQFSLKLSSVTAAD |
| | 2017/205742 A1; | TAVYYCARLDYWGQGTTVTVSS (SEQ ID NO: 59) |
| | VL: SEQ ID NO: 163 | VL: |
| | of PCT Application | DAVMTQTPLSLSVTPGQPASISCRSSQSLENTNGNTFLNWFLQKP |
| | NO: WO | GQSPQLLIYRVSNRFSGVPDRFSGSGSGTDFTLKISRVEAEDVGV |
| | 2017/205742 A1 | YFCLQVTHVPFTFGQGTKLEIK (SEQ ID NO: 58) |
| CD40 | huAb9-3 VH: SEQ | VH: |
| | ID NO: 118 of PCT | EVQLQESGPGLVKPSETLSLTCTVSGYSISSNYYWNWIRQPPGKG |
| | Application NO: WO | LEWMGYIRYDGSNNYNPSLKSRVTISRDTSKNQFSLKLSSVTAAD |
| | 2017/205742 A1; | TAVYYCARLDYWGQGTTVTVSS (SEQ ID NO: 60) |
| | VL: SEQ ID NO: 163 | VL: |
| | of PCT Application | DAVMTQTPLSLSVTPGQPASISCRSSQSLENTNGNTFLNWFLQKP |
| | NO: WO | GQSPQLLIYRVSNRFSGVPDRFSGSGSGTDFTLKISRVEAEDVGV |
| | 2017/205742 A1 | YFCLQVTHVPFTFGQGTKLEIK (SEQ ID NO: 58) |
| CD40 | huAb9-4 VH: SEQ | VH: |
| | ID NO: 116 of PCT | EVQLQESGPGLVKPSETLSLTCTVSGYSITSNYYWNWIRQPPGKG |
| | Application NO: WO | LEWMGYIRYDGSNNYNPSLKNRITISRDTSKNQFSLKLSSVTAAD |
| | 2017/205742 A1; | TAVYYCARLDYWGQGTTVTVSS (SEQ ID NO: 57) |
| | VL: SEQ ID NO: 164 | VL: |
| | of PCT Application | DAVMTQTPLSLSVTPGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 61) |
| CD40 | huAb9-5 VH: SEQ | VH: |
| | ID NO: 117 of PCT | EVQLQESGPGLVKPSETLSLTCTVSGYSITSNYYWNWIRQPPGKG |
| | Application NO: WO | LEWMGYIRYDGSNNYNPSLKNRVTISRDTSKNQFSLKLSSVTAAD |
| | 2017/205742 A1; | TAVYYCARLDYWGQGTTVTVSS (SEQ ID NO: 59) |
| | VL: SEQ ID NO: 164 | VL: |
| | of PCT Application | DAVMTQTPLSLSVTPGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 61) |
| CD40 | huAb9-6 VH: SEQ | VH: |
| | ID NO: 118 of PCT | EVQLQESGPGLVKPSETLSLTCTVSGYSISSNYYWNWIRQPPGKG |
| | Application NO: WO | LEWMGYIRYDGSNNYNPSLKSRVTISRDTSKNQFSLKLSSVTAAD |
| | 2017/205742 A1; | TAVYYCARLDYWGQGTTVTVSS (SEQ ID NO: 60) |
| | | VL: |
| | VL: SEQ ID NO: 164 | DAVMTQTPLSLSVTPGQPASISCRSSQSLENTNGNTFLNWYLQKP |
| | of PCT Application | GQSPQLLIYRVSNRFSGVPDRFSGSGSGTDFTLKISRVEAEDVGV |
| | NO: WO | YYCLQVTHVPFTFGQGTKLEIK (SEQ ID NO: 61) |
| | 2017/205742 A1 | |
| CD40 | huAb9-7 VH: SEQ | VH: |
| | ID NO: 119 of PCT | EVQLVESGGGLVKPGETLSLTCTVSGYSITSNYYWNWIRQPPGKG |
| | Application NO: WO | LEWMGYIRYDGSNNYNPSLKGRVTISRDTSKNQFYLKLSSVTAAD |
| | 2017/205742 A1; | TAVYYCARLDYWGQGTTVTVSS (SEQ ID NO: 62) |
| | VL: SEQ ID NO: 165 | VL: |
| | of PCT Application | DAVMTQTPLSLSVTEGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 63) |
| CD40 | huAb9-8 VH: SEQ | VH: |
| | ID NO: 120 of PCT | EVQLVESGGGLVQPGGSLRLSCAASGYSITSNYYWNWIRQPPGKG |
| | Application NO: WO | LEWMGYIRYDGSNNYNPSLKGRVTISRDTSKNQLYLKLSSVTAAD |
| | 2017/205742 A1; | TAVYYCARLDYWGQGTLVTVSS (SEQ ID NO: 64) |
| | VL: SEQ ID NO: 165 | VL: |
| | of PCT Application | DAVMTQTPLSLSVTEGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 63) |
| CD40 | huAb9-9 VH: SEQ | VH: |
| | ID NO: 121 of PCT | EVQLVESGGGLVKPGETLILTCTVSGYDITSNYYWNWIRQPPGKG |
| | Application NO: WO | LEWMGYIRYDGSNNYNPSLKGRVTISRDTSKNQFYLKLSSVTAAD |
| | 2017/205742 A1; | TAVYYCARLDYWGQGTTVTVSS (SEQ ID NO: 65) |
| | VL: SEQ ID NO: 166 | VL: |
| | of PCT Application | DAVMTQTPLSLAVLPGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 66) |
| CD40 | huAb9 rehu#1 VH: | VH: |
| | SEQ ID NO: 122 of | QVQLQESGPGLVKPSETLSLTCTVSGYSITSNYYWNWIRQPPGKG |
| | PCT Application NO: | LEWMGYIRYDGSNNYNPSLKNRITISRDTSKNQFSLKLSSVTAAD |
| | WO 2017/205742 | TAVYYCARLDYWGQGTLVTVSS (SEQ ID NO: 67) |
| | A1; VL: SEQ ID | VL: |
| | NO: 167 of PCT | DIQMTQSPSSLSASVGDRVTITCRSSQSLENTNGNTFLNWYQQKP |
| | Application NO: WO | GKAPKLLIYRVSNRFSGVPSRFSGSGSGTDFTLTISSLQPEDFAT |
| | 2017/205742 A1 | YYCLQVTHVPFTFGQGTKVEIK (SEQ ID NO: 68) |
| CD40 | huAb9 rehu#2 VH: | VH: |
| | SEQ ID NO: 122 of | QVQLQESGPGLVKPSETLSLTCTVSGYSITSNYYWNWIRQPPGKG |
| | PCT Application NO: | LEWMGYIRYDGSNNYNPSLKNRITISRDTSKNQFSLKLSSVTAAD |
| | WO 2017/205742 | TAVYYCARLDYWGQGTLVTVSS (SEQ ID NO: 67) |
| | A1; VL: SEQ ID | VL: |
| | NO: 168 of PCT | DAVMTQSPLSLPVTLGEPASISCRSSQSLENTNGNTFLNWFQQKP |
| | Application NO: WO | GQSPRLLIYRVSNRFSGVPDRFSGSGSGTDFTLKISRVEAEDVGV |
| | 2017/205742 A1 | YYCLQVTHVPFTFGQGTKLEIK (SEQ ID NO: 69) |
| CD40 | huAb9 rehu#3 VH: | VH: |
| | SEQ ID NO: 123 of | EVQLVESGGGLVQPGGSLRLSCAASGYSITSNYYWNWVRQAPGKG |
| | PCT Application NO: | LEWMGYIRYDGSNNYNPSLKNRITISRDTSKNTFYLQMNSLRAED |
| | WO 2017/205742 | TAVYYCARLDYWGQGTLVTVSS (SEQ ID NO: 70) |
| | A1; VL: SEQ ID | VL: |
| | NO: 169 of PCT | DAQMTQSPSSLSASVGDRVTITCRSSQSLENTNGNTFLNWFQQKP |
| | Application NO: WO | GKAPKLLIYRVSNRFSGVPSRFSGSGSGTDFTLTISSLQPEDFAT |
| | 2017/205742 A1 | YYCLQVTHVPFTFGQGTKLEIK (SEQ ID NO: 71) |
| CD40 | huAb9 A21 VH: SEQ | VH: |
| | ID NO: 117 of PCT | EVQLQESGPGLVKPSETLSLTCTVSGYSITSNYYWNWIRQPPGKG |
| | Application NO: WO | LEWMGYIRYDGSNNYNPSLKNRVTISRDTSKNQFSLKLSSVTAAD |
| | 2017/205742 A1; | TAVYYCARLDYWGQGTTVTVSS (SEQ ID NO: 59) |
| | VL: SEQ ID NO: 170 | VL: |
| | of PCT Application | DIVMTQTPLSLSVTPGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 72) |
| CD40 | huAb9 A21 VH: SEQ | VH: |
| | ID NO: 117 of PCT | EVQLQESGPGLVKPSETLSLTCTVSGYSITSNYYWNWIRQPPGKG |
| | Application NO: WO | LEWMGYIRYDGSNNYNPSLKNRVTISRDTSKNQFSLKLSSVTAAD |
| | 2017/205742 A1; | TAVYYCARLDYWGQGTTVTVSS (SEQ ID NO: 59) |
| | VL: SEQ ID NO: 171 | VL: |
| | of PCT Application | DVVMTQTPLSLSVTPGQPASISCRSSQSLENTNGNTFLNWYLQKP |
| | NO: WO | GQSPQLLIYRVSNRFSGVPDRFSGSGSGTDFTLKISRVEAEDVGV |
| | 2017/205742 A1 | YYCLQVTHVPFTFGQGTKLEIK (SEQ ID NO: 73) |
| OX40 | VH: SEQ ID NO: 56 | VH: |
| | of PCT Application | EVQLVQSGAEVKKPGASVKVSCKASGYTFTDSYMSWVRQAPGQGL |
| | NO: WO | EWIGDMYPDNGDSSYNQKFRERVTITRDTSTSTAYLELSSLRSED |
| | 2015/153513 A1; | TAVYYCVLAPRWYFSVWGQGTLVTVSS (SEQ ID NO: 74) |
| | VL: SEQ ID NO: 57 | VL: |
| | of PCT Application | DIQMTQSPSSLSASVGDRVTITCRASQDISNYLNWYQQKPGKAPK |
| | NO: WO | LLIYYTSRLRSGVPSRFSGSGSGTDFTLTISSLQPEDFATYYCQQ |
| | 2015/153513 A1 | GHTLPPTFGQGTKVEIK (SEQ ID NO: 75) |
| OX40 | VH: SEQ ID NO: 94 | VH: |
| | of PCT Application | EVQLVQSGAEVKKPGASVKVSCKASGYTFTDSYMSWVRQAPGQGL |
| | NO: WO | EWIGDMYPDNGDSSYNQKFRERVTITRDTSTSTAYLELSSLRSED |
| | 2015/153513 A1; | TAVYYCVLAPRWYFSVWGQGTLVTVSS (SEQ ID NO: 74) |
| | VL: SEQ ID NO: 95 | VL: |
| | of PCT Application | DIQMTQSPSSLSASVGDRVTITCRASQDISNYLNWYQQKPGKAPK |
| | NO: WO | LLIYYTSRLRSGVPSRFSGSGSGTDFTLTISSLQPEDFATYYCQQ |
| | 2015/153513 A1 | AHTLPPTFGQGTKVEIK (SEQ ID NO: 76) |
| OX40 | VH: SEQ ID NO: 96 | VH: |
| | of PCT Application | EVQLVQSGAEVKKPGASVKVSCKASGYTFTDSYMSWVRQAPGQGL |
| | NO: WO | EWIGDMYPDNGDSSYNQKFRERVTITRDTSTSTAYLELSSLRSED |
| | 2015/153513 A1; | TAVYYCVLAPRWYFSVWGQGTLVTVSS (SEQ ID NO: 74) |
| | VL: SEQ ID NO: 97 | VL: |
| | of PCT Application | DIQMTQSPSSLSASVGDRVTITCRASQDISNYLNWYQQKPGKAPK |
| | NO: WO | LLIYYTSRLRSGVPSRFSGSGSGTDFTLTISSLQPEDFATYYCQQ |
| | 2015/153513 A1 | GHTLAPTFGQGTKVEIK (SEQ ID NO: 77) |
| OX40 | VH: SEQ ID NO: 180 | VH: |
| | of PCT Application | EVQLQQSGPELVKPGASVKISCKASGYTFTDSYMSWVKQSHGKTL |
| | NO: WO | EWIGDMYPDNGDSSYNQKFREKVTLTVDKSSTTAYMEFRSLTSED |
| | 2015/153513 A1; | SAVYYCVLAPRWYFSVWGTGTTVTVSS (SEQ ID NO: 78) |
| | VL: SEQ ID NO: 179 | VL: |
| | of PCT Application | DIQMTQTTSSLSASLGDRVTISCRASQDISNYLNWYQQKPDGTVK |
| | NO: WO | LLIYYTSRLRSGVPSRFSGSGSGKDYFLTISNLEQEDVAAYFCQQ |
| | 2015/153513 A1 | GHTLPPTFGGGTKLEIK (SEQ ID NO: 79) |
| OX40 | VH: SEQ ID NO: 182 | VH: |
| | of PCT Application | QVQLQQSGAELVRPGTSVKVSCKASGYAFTNYLIEWVKQRPGQGL |
| | NO: WO | EWIGVINPGSGDTYYSEKFKGKVTLTADKSSSTAYMQLSSLTSED |
| | 2015/153513 A1; | SAVYFCARDRLDYWGQGTTLTVSS (SEQ ID NO: 80) |
| | VL: SEQ ID NO: 181 | VL: |
| | of PCT Application | DILMTQSPSSMSVSLGDTVSITCHASQDISSYIVWLQQKPGKSFR |
| | NO: WO | GLIYHGTNLEDGIPSRFSGSGSGADYSLTISSLESEDFADYYCVH |
| | 2015/153513 A1 | YAQFPYTFGGGTKLEIK (SEQ ID NO: 81) |
| OX40 | VH: SEQ ID NO: 36 | VH: |
| | of PCT Application | EVQLVESGGGLVQPGGSLRLSCAASGFTFSSYSMNWVRQAPGKGL |
| | NO: WO | EWVSYISSSSSTIDYADSVKGRFTISRDNAKNSLYLQMNSLRDED |
| | 2015/095423 A2; | TAVYYCARESGWYLFDYWGQGTLVTVSS (SEQ ID NO: 82) |
| | VL: SEQ ID NO: 37 | VL: |
| | of PCT Application | DIQMTQSPSSLSASVGDRVTITCRASQGISSWLAWYQQKPEKAPK |
| | NO: WO | SLIYAASSLQSGVPSRFSGSGSGTDFTLTISSLQPEDFATYYCQQ |
| | 2015/095423A2 | YNSYPPTFGGGTKVEIK (SEQ ID NO: 83) |
| OX40 | VH: SEQ ID NO: 38 | VH: |
| | of PCT Application | EVQLVESGGGLVQPGRSLRLSCAASGFTFDDYAMHWVRQAPGKGL |
| | NO: WO | EWVSGISWNSGSIGYADSVKGRFTISRDNAKNSLYLQMNSLRAED |
| | 2015/095423 A2; | TALYYCAKDQSTADYYFYYGMDVWGQGTTVTVSS (SEQ ID |
| | VL: SEQ ID NO: 39 | NO: 84) |
| | of PCT Application | VL: |
| | NO: WO | EIVVTQSPATLSLSPGERATLSCRASQSVSSYLAWYQQKPGQAPR |
| | 2015/095423A2 | LLIYDASNRATGIPARFSGSGSGTDFTLTISSLEPEDFAVYYCQQ |
| | | RSNWPTFGQGTKVEIK (SEQ ID NO: 85) |
| OX40 | VH: SEQ ID NO: 40 | VH: |
| | of PCT Application | QVQLVQSGSELKKPGASVKVSCKASGYTFTDYSMHWVRQAPGQGL |
| | NO: WO | KWMGWINTETGEPTYADDFKGRFVFSLDTSVSTAYLQISSLKAED |
| | 2015/095423 A2; | TAVYYCANPYYDYVSYYAMDYWGQGTTVTVSS (SEQ ID NO: |
| | VL: SEQ ID NO: 41 | 86) |
| | of PCT Application | VL: |
| | NO: WO | DIQMTQSPSSLSASVGDRVTITCKASQDVSTAVAWYQQKPGKAPK |
| | 2015/095423A2 | LLIYSASYLYTGVPSRFSGSGSGTDFTFTISSLQPEDIATYYCQQ |
| | | HYSTPRTFGQGTKLEIK (SEQ ID NO: 87) |
| OX40 | VH: SEQ ID NO: 42 | VH: |
| | of PCT Application | EVQLVESGGGLVQPGGSLRLSCAASEYEFPSHDMSWVRQAPGKGL |
| | NO: WO | ELVAAINSDGGSTYYPDTMERRFTISRDNAKNSLYLQMNSLRAED |
| | 2015/095423 A2; | TAVYYCARHYDDYYAWFAYWGQGTMVTVSS (SEQ ID NO: |
| | VL: SEQ ID NO: 43 | 88) |
| | of PCT Application | VL: |
| | NO: WO | EIVLTQSPATLSLSPGERATLSCRASKSVSTSGYSYMHWYQQKPG |
| | 2015/095423A2 | QAPRLLIYLASNLESGVPARFSGSGSGTDFTLTISSLEPEDFAVY |
| | | YCQHSRELPLTFGGGTKVEIK (SEQ ID NO: 89) |
| OX40 | VH: SEQ ID NO: 61 | VH: |
| | of PCT Application | MYLGLNYVFIVFLLNGVQSEVKLEESGGGLVQPGGSMKLSCAASG |
| | NO: WO | FTFSDAWMDWVRQSPEKGLEWVAEIRSKANNHATYYAESVNGRFT |
| | 2015/095423 A2; | ISRDDSKSSVYLQMNSLRAEDTGIYYCTWGEVFYFDYWGQGTTLT |
| | VL: SEQ ID NO: 62 | VSS (SEQ ID NO: 90) |
| | of PCT Application | VL: |
| | NO: WO | MRPSIQFLGLLLFWLHGAQCDIQMTQSPSSLSASLGGKVTITCKS |
| | 2015/095423A2 | SQDINKYIAWYQHKPGKGPRLLIHYTSTLQPGIPSRFSGSGSGRD |
| | | YSFSISNLEPEDIATYYCLQYDNLLTFGAGTKLELK (SEQ ID |
| | | NO: 91) |
| OX40 | VH: SEQ ID NO: 46 | VH: |
| | of PCT Application | EVQLQQSGPELVKPGASVKMSCKASGYTFTSYVMHWVKQKPGQGL |
| | NO: WO | EWIGYINPYNDGTKYNEKFKGKATLTSDKSSSTAYMELSSLTSED |
| | 2015/095423 A2; | SAVYYCANYYGSSLSMDYWGQGTSVTVSS (SEQ ID NO: 92) |
| | VL: SEQ ID NO: 47 | VL: |
| | of PCT Application | DIQMTQTTSSLSASLGDRVTISCRASQDISNYLNWYQQKPDGTVK |
| | NO: WO | LLIYYTSRLHSGVPSRFSGSGSGTDYSLTISNLEQEDIATYFCQQ |
| | 2015/095423A2 | GNTLPWTFGGGTKLEIKR (SEQ ID NO: 93) |
| OX40 | VH: SEQ ID NO: 48 | VH: |
| | of PCT Application | EVQLQQSGPELVKPGASVKISCKTSGYTFKDYTMHWVKQSHGKSL |
| | NO: WO | EWIGGIYPNNGGSTYNQNFKDKATLTVDKSSSTAYMEFRSLTSED |
| | 2015/095423 A2; | SAVYYCARMGYHGPHLDFDVWGAGTTVTVSP (SEQ ID NO: |
| | VL: SEQ ID NO: 49 | 94) |
| | of PCT Application | VL: |
| | NO: WO | DIVMTQSHKFMSTSLGDRVSITCKASQDVGAAVAWYQQKPGQSPK |
| | 2015/095423A2 | LLIYWASTRHTGVPDRFTGGGSGTDFTLTISNVQSEDLTDYFCQQ |
| | | YINYPLTFGGGTKLEIKR (SEQ ID NO: 95) |
| OX40 | VH: SEQ ID NO: 50 | VH: |
| | of PCT Application | QVQLVQSGAEVKKPGASVKVSCKASGYTFTSYVMHWVRQAPGQRL |
| | NO: WO | EWMGYINPYNDGTKYNEKFKGRVTITSDTSASTAYMELSSLRSED |
| | 2015/095423 A2; | TAVYYCANYYGSSLSMDYWGQGTLVTVSS (SEQ ID NO: 96) |
| | VL: SEQ ID NO: 52 | VL: |
| | of PCT Application | DIQMTQSPSSLSASVGDRVTITCRASQDISNYLNWYQQKPGKAVK |
| | NO: WO | LLIYYTSRLHSGVPSRFSGSGSGTDYTLTISSLQPEDFATYFCQQ |
| | 2015/095423A2 | GNTLPWTFGQGTKVEIKR (SEQ ID NO: 97) |
| OX40 | VH: SEQ ID NO: 53 | VH: |
| | of PCT Application | QVQLVQSGAEVKKPGASVKVSCKASGYTFTSYVMHWVRQAPGQRL |
| | NO: WO | EWIGYINPYNDGTKYNEKFKGRATITSDTSASTAYMELSSLRSED |
| | 2015/095423 A2; | TAVYYCANYYGSSLSMDYWGQGTLVTVSS (SEQ ID NO: 98) |
| | VL: SEQ ID NO: 51 | VL: |
| | of PCT Application | DIQMTQSPSSLSASVGDRVTITCRASQDISNYLNWYQQKPGKAPK |
| | NO: WO | LLIYYTSRLHSGVPSRFSGSGSGTDYTLTISSLQPEDFATYYCQQ |
| | 2015/095423A2 | GNTLPWTFGQGTKVEIKR (SEQ ID NO: 99) |
| OX40 | VH: SEQ ID NO: 53 | VH: |
| | of PCT Application | QVQLVQSGAEVKKPGASVKVSCKASGYTFTSYVMHWVRQAPGQRL |
| | NO: WO | EWIGYINPYNDGTKYNEKFKGRATITSDTSASTAYMELSSLRSED |
| | 2015/095423 A2; | TAVYYCANYYGSSLSMDYWGQGTLVTVSS (SEQ ID NO: 98) |
| | VL: SEQ ID NO: 52 | VL: |
| | of PCT Application | DIQMTQSPSSLSASVGDRVTITCRASQDISNYLNWYQQKPGKAVK |
| | NO: WO | LLIYYTSRLHSGVPSRFSGSGSGTDYTLTISSLQPEDFATYFCQQ |
| | 2015/095423A2 | GNTLPWTFGQGTKVEIKR (SEQ ID NO: 97) |
| OX40 | VH: SEQ ID NO: 54 | VH: |
| | of PCT Application | QVQLVQSGAEVKKPGASVKVSCKASGYTFTSYVMHWVRQAPGQRL |
| | NO: WO | EWIGYINPYNDGTKYNEKFKGRATLTSDKSASTAYMELSSLRSED |
| | 2015/095423 A2; | TAVYYCANYYGSSLSMDYWGQGTLVTVSS (SEQ ID NO: |
| | VL: SEQ ID NO: 51 | 100) |
| | of PCT Application | VL: |
| | NO: WO | DIQMTQSPSSLSASVGDRVTITCRASQDISNYLNWYQQKPGKAPK |
| | 2015/095423A2 | LLIYYTSRLHSGVPSRFSGSGSGTDYTLTISSLQPEDFATYYCQQ |
| | | GNTLPWTFGQGTKVEIKR (SEQ ID NO: 99) |
| OX40 | VH: SEQ ID NO: 54 | VH: |
| | of PCT Application | QVQLVQSGAEVKKPGASVKVSCKASGYTFTSYVMHWVRQAPGQRL |
| | NO: WO | EWIGYINPYNDGTKYNEKFKGRATLTSDKSASTAYMELSSLRSED |
| | 2015/095423 A2; | TAVYYCANYYGSSLSMDYWGQGTLVTVSS (SEQ ID NO: |
| | VL: SEQ ID NO: 52 | 100) |
| | of PCT Application | VL |
| | NO: WO | DIQMTQSPSSLSASVGDRVTITCRASQDISNYLNWYQQKPGKAVK |
| | 2015/095423A2 | LLIYYTSRLHSGVPSRFSGSGSGTDYTLTISSLQPEDFATYFCQQ |
| | | GNTLPWTFGQGTKVEIKR (SEQ ID NO: 97) |
| OX40 | VH: SEQ ID NO: 55 | VH: |
| | of PCT Application | QVQLVQSGAEVKKPGSSVKVSCKASGYTFKDYTMHWVRQAPGQGL |
| | NO: WO | EWMGGIYPNNGGSTYNQNFKDRVTITADKSTSTAYMELSSLRSED |
| | 2015/095423 A2; | TAVYYCARMGYHGPHLDFDVWGQGTTVTVSS (SEQ ID NO: |
| | VL: SEQ ID NO: 56 | 101) |
| | of PCT Application | VL |
| | NO: WO | DIQMTQSPSSLSASVGDRVTITCKASQDVGAAVAWYQQKPGKAPK |
| | 2015/095423A2 | LLIYWASTRHTGVPSRFSGSGSGTDFTLTISSLQPEDFATYYCQQ |
| | | YINYPLTFGGGTKVEIKR (SEQ ID NO: 102) |
| OX40 | VH: SEQ ID NO: 55 | VH: |
| | of PCT Application | QVQLVQSGAEVKKPGSSVKVSCKASGYTFKDYTMHWVRQAPGQGL |
| | NO: WO | EWMGGIYPNNGGSTYNQNFKDRVTITADKSTSTAYMELSSLRSED |
| | 2015/095423 A2; | TAVYYCARMGYHGPHLDFDVWGQGTTVTVSS (SEQ ID NO: |
| | VL: SEQ ID NO: 57 | 101) |
| | of PCT Application | VL: |
| | NO: WO | DIQMTQSPSSLSASVGDRVTITCKASQDVGAAVAWYQQKPGKAPK |
| | 2015/095423A2 | LLIYWASTRHTGVPDRFSGGGSGTDFTLTISSLQPEDFATYYCQQ |
| | | YINYPLTFGGGTKVEIKR (SEQ ID NO: 103) |
| OX40 | VH: SEQ ID NO: 58 | VH: |
| | of PCT Application | QVQLVQSGAEVKKPGSSVKVSCKASGYTFKDYTMHWVRQAPGQGL |
| | NO: WO | EWIGGIYPNNGGSTYNQNFKDRVTLTADKSTSTAYMELSSLRSED |
| | 2015/095423 A2; | TAVYYCARMGYHGPHLDFDVWGQGTTVTVSS (SEQ ID NO: |
| | VL: SEQ ID NO: 56 | 104) |
| | of PCT Application | VL: |
| | NO: WO | DIQMTQSPSSLSASVGDRVTITCKASQDVGAAVAWYQQKPGKAPK |
| | 2015/095423A2 | LLIYWASTRHTGVPSRFSGSGSGTDFTLTISSLQPEDFATYYCQQ |
| | | YINYPLTFGGGTKVEIKR (SEQ ID NO: 102) |
| OX40 | VH: SEQ ID NO: 58 | VH: |
| | of PCT Application | QVQLVQSGAEVKKPGSSVKVSCKASGYTFKDYTMHWVRQAPGQGL |
| | NO: WO | EWIGGIYPNNGGSTYNQNFKDRVTLTADKSTSTAYMELSSLRSED |
| | 2015/095423 A2; | TAVYYCARMGYHGPHLDFDVWGQGTTVTVSS (SEQ ID NO: |
| | VL: SEQ ID NO: 57 | 104) |
| | of PCT Application | VL: |
| | NO: WO | DIQMTQSPSSLSASVGDRVTITCKASQDVGAAVAWYQQKPGKAPK |
| | 2015/095423A2 | LLIYWASTRHTGVPDRFSGGGSGTDFTLTISSLQPEDFATYYCQQ |
| | | YINYPLTFGGGTKVEIKR (SEQ ID NO: 103) |
| OX40 | VH: SEQ ID NO: 59 | VH: |
| | of PCT Application | QVQLVQSGAEVKKPGSSVKVSCKASGYTFKDYTMHWVRQAPGQGL |
| | NO: WO | EWIGGIYPNNGGSTYNQNFKDRATLTVDKSTSTAYMELSSLRSED |
| | 2015/095423 A2; | TAVYYCARMGYHGPHLDFDVWGQGTTVTVSS (SEQ ID NO: |
| | VL: SEQ ID NO: 56 | 105) |
| | of PCT Application | VL: |
| | NO: WO | DIQMTQSPSSLSASVGDRVTITCKASQDVGAAVAWYQQKPGKAPK |
| | 2015/095423A2 | LLIYWASTRHTGVPSRFSGSGSGTDFTLTISSLQPEDFATYYCQQ |
| | | YINYPLTFGGGTKVEIKR (SEQ ID NO: 102) |
| OX40 | VH: SEQ ID NO: 59 | VH: |
| | of PCT Application | QVQLVQSGAEVKKPGSSVKVSCKASGYTFKDYTMHWVRQAPGQGL |
| | NO: WO | EWIGGIYPNNGGSTYNQNFKDRATLTVDKSTSTAYMELSSLRSED |
| | 2015/095423 A2; | TAVYYCARMGYHGPHLDFDVWGQGTTVTVSS (SEQ ID NO: |
| | VL: SEQ ID NO: 57 | 105) |
| | of PCT Application | VL: |
| | NO: WO | DIQMTQSPSSLSASVGDRVTITCKASQDVGAAVAWYQQKPGKAPK |
| | 2015/095423A2 | LLIYWASTRHTGVPDRFSGGGSGTDFTLTISSLQPEDFATYYCQQ |
| | | YINYPLTFGGGTKVEIKR (SEQ ID NO: 103) |
| OX40 | VH: SEQ ID NO: 7 of | VH: |
| | PCT Application NO: | EVQLVESGGGLVQPGGSLRLSCAASGFTFSSYSMNWVRQAPGKGL |
| | WO 2017/130076 | EWVSYISSSSSTIDYADSVKGRFTISRDNAKNSLYLQMNSLRDED |
| | A1; VL: SEQ ID | TAVYYCARESGWYLFDYWGQGTLVTVSS (SEQ ID NO: 82) |
| | NO: 8 of PCT | VL: |
| | Application NO: WO | DIQMTQSPSSLSASVGDRVTITCRASQGISSWLAWYQQKPEKAPK |
| | 2017/130076 A1 | SLIYAASSLQSGVPSRFSGSGSGTDFTLTISSLQPEDFATYYCQQ |
| | | YNSYPPTFGGGTKVEIK (SEQ ID NO: 83) |
| GITR | VH: SEQ ID NO: 1 of | VH: |
| | U.S. Application NO: | QVQLVQSGAEVKKPGASVKVSCKASGYTFTNYHLHWVRQAPGQGL |
| | US 2022/0106398 | EWMGMINPNDGSTTYAQKFQGRVTMTRDTSTSTVYMELSSLRSED |
| | A1; VL: SEQ ID | TAVYYCARSTYYYDSSGYYYYYYGMDVWGQGTTVTVSS (SEQ |
| | NO: 5 of U.S. | ID NO: 106) |
| | Application NO: US | VL: |
| | 2022/0106398 A1 | EIVMTQSPATLSVSPGERATLSCRASQSVYSNYLAWYQQKPGQAP |
| | | RLLIYGASTRATGIPARFSGSGSGTEFTLTISSLQSEDFAVYYCQ |
| | | QYHSYPLTFGGGTKVEIK (SEQ ID NO: 107) |
| GITR | VH: SEQ ID NO: 9 of | VH: |
| | U.S. Application NO: | QVQLVQSGAEVKKPGSSVKVSCKASGYRFTGYHLHWVRQAPGQGL |
| | US 2022/0106398 | EWMGGIIPIFGTANYAQKFQGRVTITADKSTSTAYMELSSLRSED |
| | A1; VL: SEQ ID | TAVYYCAYGVPPDPWGQGTLVTVSS (SEQ ID NO: 108) |
| | NO: 13 of U.S. | VL: |
| | Application NO: US | DIVMTQSPLSLPVTPGEPASISCRSSQSLLHSNGYNYLDWYLQKP |
| | 2022/0106398 A1 | GQSPQLLIYLGSYRASGVPDRFSGSGSGTDFTLKISRVEAEDVGV |
| | | YYCRQALRTPLTFGGGTKVEIK (SEQ ID NO: 109) |
| GITR | VH: SEQ ID NO: 17 | VH: |
| | of U.S. Application | QVQLVQSGAEVKKPGASVKVSCKASGYTFTSYDINWVRQAPGQGL |
| | NO: US | EWMGWISGYNGNTNYAQKFQGRVTMTRDTSTSTVYMELSSLRSED |
| | 2022/0106398 A1; | TAVYYCARSHEYYYYYGMDVWGQGTLVTVSS (SEQ ID NO: |
| | VL: SEQ ID NO: 21 | 110) |
| | of U.S. Application | VL: |
| | NO: US | EIVMTQSPATLSVSPGERATLSCRASQSVSSSYLAWYQQKPGQAP |
| | 2022/0106398 A1 | RLLIYGASTRATGIPARFSGSGSGTEFTLTISSLQSEDFAVYYCQ |
| | | QYYTTPFTFGPGTKVDIK (SEQ ID NO: 111) |
| GITR | VH: SEQ ID NO: 25 | VH: |
| | of U.S. Application | EVQLLESGGGLVQPGGSLRLSCAASGFTFSSYDMHWVRQAPGKGL |
| | NO: US | EWVSAISSSGGSIYYADSVKGRFTISRDNSKNTLYLQMNSLRAED |
| | 2022/0106398 A1; | TAVYYCARDSVVVPKGPNRKYYYYGMDVWGQGTKVTVSS (SEQ |
| | VL: SEQ ID NO: 29 | ID NO: 112) |
| | of U.S. Application | VL: |
| | NO: US | DIVMTQSPLSLPVTPGEPASISCRSSQSLLHSNGYNYLDWYLQKP |
| | 2022/0106398 A1 | GQSPQLLIYLGSNRASGVPDRFSGSGSGTDFTLKISRVEAEDVGV |
| | | YYCMQATHSPYTFGQGTKVEIK (SEQ ID NO: 113) |
| GITR | VH: SEQ ID NO: 33 | VH: |
| | of U.S. Application | EVQLLESGGGLVQPGGSLRLSCAASGFTFDAYAMHWVRQAPGKGL |
| | NO: US | EWVSAIGTGGDTYYADSVKGRFTISRDNSKNTLYLQMNSLRAEDT |
| | 2022/0106398 A1; | AVYYCARDLYGSGSPQYYYYYGMDVWGQGTTVTVSS (SEQ ID |
| | VL: SEQ ID NO: 37 | NO: 114) |
| | of U.S. Application | VL: |
| | NO: US | DIQMTQSPSSLSASVGDRVTITCRASQGIKNDLGWYQQKPGKAPK |
| | 2022/0106398 A1 | LLIYAASSLQSGVPSRFSGSGSGTDFTLTISSLQPEDFATYYCQQ |
| | | SYSVPFTFGQGTKVEIK (SEQ ID NO: 115) |
| GITR | VH: SEQ ID NO: 41 | VH: |
| | of U.S. Application | QVQLVQSGAEVKKPGASVKVSCKASGYAFTAYYLHWVRQAPGQGL |
| | NO: US | EWMGWMNPNSGNTGYAQKFQGRVTMTRDTSTSTVYMELSSLRSED |
| | 2022/0106398 A1; | TAVYYCAREGWGYYDGGFDPWGQGTLVTVSS (SEQ ID NO: |
| | VL: SEQ ID NO: 45 | 116) |
| | of U.S. Application | VL: |
| | NO: US | DIVMTQSPLSLPVTPGEPASISCRSSQSLLHSNGYNYLDWYLQKP |
| | 2022/0106398 A1 | GQSPQLLIYLGSRRASGVPDRFSGSGSGTDFTLKISRVEAEDVGV |
| | | YYCMQGSHWPPTFGPGTKVDIK (SEQ ID NO: 117) |
| GITR | VH: SEQ ID NO: 49 | VH: |
| | of U.S. Application | QVQLVQSGAEVKKPGASVKVSCKASGGTSSTYAVTWVRQAPGQGL |
| | NO: US | EWMGVINPNDGSTTYAQNFQGRVTMTRDTSTSTVYMELSSLRSED |
| | 2022/0106398 A1; | TAVYYCARWTPLFGVVIPDYYYYGMDVWGQGTLVTVSS (SEQ |
| | VL: SEQ ID NO: 53 | ID NO: 118) |
| | of U.S. Application | VL: |
| | NO: US | DIQMTQSPSSLSASVGDRVTITCQASQDISNYLNWYQQKPGKAPK |
| | 2022/0106398 A1 | LLIYAASSLQSGVPSRFSGSGSGTDFTLTISSLQPEDFATYYCQQ |
| | | SYSTPPTFGQGTRLEIK (SEQ ID NO: 119) |
| GITR | VH: SEQ ID NO: 57 | VH: |
| | of U.S. Application | QVQLVQSGAEVKKPGASVKVSCKASGGTFSSYVISWVRQAPGQGL |
| | NO: US | EWMGWMNPGSGNTGYAQKFQGRVTMTRDTSTSTVYMELSSLRSED |
| | 2022/0106398 A1; | TAVYYCITDSEDDYWGQGTLVTVSS (SEQ ID NO: 120) |
| | VL: SEQ ID NO: 61 | VL: |
| | of U.S. Application | DIVMTQSPDSLAVSLGERATINCKSSQSVLYSSNNKNYLAWYQQK |
| | NO: US | PGQPPKLLIYWASTRESGVPDRFSGSGSGTDFTLTISSLQAEDVA |
| | 2022/0106398 A1 | VYYCQQYYNTPYTFGQGTKVEIK (SEQ ID NO: 121) |
| GITR | VH: SEQ ID NO: 65 | VH: |
| | of U.S. Application | QVQLVQSGAEVKKPGASVKVSCKASGHTFTSQYMHWVRQAPGQGL |
| | NO: US | EWVGVINPNDGSTSYAQKFQGRVTMTRDTSTSTVYMELSSLRSED |
| | 2022/0106398 A1; | TAVYYCARDGAYYYDSSGYYRSSNFDYWGQGTLVTVSS (SEQ |
| | VL: SEQ ID NO: 69 | ID NO: 122) |
| | of U.S. Application | VL: |
| | NO: US | DIQMTQSPSSLSASVGDRVTITCQASQDIGNYLNWYQQKPGKAPK |
| | 2022/0106398 A1 | LLIYGASNLETGVPSRFSGSGSGTDFTLTISSLQPEDFATYYCQQ |
| | | GYSFPLTFGQGTKVEIK (SEQ ID NO: 123) |
| GITR | VH: SEQ ID NO: 73 | VH: |
| | of U.S. Application | QVQLVQSGAEVKKPGASVKVSCKASGYTFTSNYMHWVRQAPGQGL |
| | NO: US | EWMGWMNPNSGNTAYAQKFQGRVTMTRDTSTSTVYMELSSLRSED |
| | 2022/0106398 A1; | TAVYYCARGRGGQLLFDYWGQGTLVTVSS (SEQ ID NO: |
| | VL: SEQ ID NO: 77 | 124) |
| | of U.S. Application | VL: |
| | NO: US | DIQMTQSPSSLSASVGDRVTITCRASQGISSWLAWYQQKPGKAPK |
| | 2022/0106398 A1 | LLIYAASTLQSGVPSRFSGSGSGTDFTLTISSLQPEDFATYYCQQ |
| | | YNSYSTFGQGTKVEIK (SEQ ID NO: 125) |
| GITR | VH: SEQ ID NO: 81 | VH: |
| | of U.S. Application | QVQLVQSGAEVKKPGASVKVSCKASGGTFSNYGISWVRQAPGQGL |
| | NO: US | EWMGIINPTDGSTTYAQKFQGRVTMTRDTSTSTVYMELSSLRSED |
| | 2022/0106398 A1; | TAVYYCARWWGSGWSWYFDLWGRGTLVTVSS (SEQ ID NO: |
| | VL: SEQ ID NO: 85 | 126) |
| | of U.S. Application | VL: |
| | NO: US | DIQMTQSPSSLSASVGDRVTITCRASQSISSYLNWYQQKPGKAPK |
| | 2022/0106398 A1 | LLIYAASSLESGVPSRFSGSGSGTDFTLTISSLQPEDFATYYCQQ |
| | | SYSTPPTFGQSTRLEIK (SEQ ID NO: 127) |
| GITR | VH: SEQ ID NO: 89 | VH: |
| | of U.S. Application | QVQLVQSGAEVKKPGASVKVSCKASGYTFTSYAINWVRQAPGQGL |
| | NO: US | EWMGILSPSGGGTSYAPKFQGRVTMTRDTSTSTVYMELSSLRSED |
| | 2022/0106398 A1; | TAVYYCARGPWYFDLWGRGTLVTVSS (SEQ ID NO: 128) |
| | VL: SEQ ID NO: 93 | VL: |
| | of U.S. Application | DIQMTQSPSSLSASVGDRVTITCRASQGISNSLAWYQQKPGKAPK |
| | NO: US | LLIYAASSLQSGVPSRFSGSGSGTDFTLTISSLQPEDFATYYCQQ |
| | 2022/0106398 A1 | SYSTPFTFGPGTKVDIK (SEQ ID NO: 129) |
| GITR | VH: SEQ ID NO: 97 | VH: |
| | of U.S. Application | QVQLVQSGAEVKKPGSSVKVSCKASGGTFSSYAISWVRQAPGQGL |
| | NO: US | EWMGGIVPMLGSPHYAQKFQGRVTITADESTSTAYMELSSLRSED |
| | 2022/0106398 A1; | TAVYYCARGSWLVADFQHWGQGTLVTVSS (SEQ ID NO: |
| | VL: SEQ ID NO: 101 | 130) |
| | of U.S. Application | VL: |
| | NO: US | DIVMTQSPDSLAVSLGERATINCKSSQSLLYSSNNRNYLAWYQQK |
| | 2022/0106398 A1 | PGQPPKLLIYWASTRESGVPDRFSGSGSGTDFTLTISSLQAEDVA |
| | | VYYCQQYYSTPITFGQGTRLEIK (SEQ ID NO: 131) |
| GITR | VH: SEQ ID NO: 105 | VH: |
| | of U.S. Application | EVQLLESGGGLVKPGGSLRLSCAASGFRFSVYWMSWVRQAPGKGL |
| | NO: US | EWVSGISGSGGTTYYADSVKGRFTISRDDSKNTLYLQMNSLKTED |
| | 2022/0106398 A1; | TAVYYCARVRRDGYNYNFDYWGQGTLVTVSS (SEQ ID NO: |
| | VL: SEQ ID NO: 109 | 132) |
| | of U.S. Application | VL: |
| | NO: US | DIQMTQSPSSLSASVGDRVTITCQASQDISNYLNWYQQKPGKAPK |
| | 2022/0106398 A1 | LLIFDASSLEAGVPSRFSGSGSGTDFTLTISSLQPEDFATYYCQQ |
| | | ANSFPPTFGQGTEVEIK (SEQ ID NO: 133) |
| GITR | VH: SEQ ID NO: 113 | VH: |
| | of U.S. Application | QVQLVQSGAEVKKPGSSVKVSCKASGFTFTTSAVQWVRQAPGQGL |
| | NO: US | EWMGGIIPIFGTANYAQKFQGRVTITADESTSTAYMELSSLRSED |
| | 2022/0106398 A1; | TAVYYCAKGSGYEFPGGSEYFQHWGQGTLVTVSS (SEQ ID |
| | VL: SEQ ID NO: 117 | NO: 134) |
| | of U.S. Application | VL: |
| | NO: US | DIVMTQSPDSLAVSLGERATINCKSSQSVLYNSNNLNYLAWYQQK |
| | 2022/0106398 A1 | PGQPPKLLIYWASIRESGVPDRFSGSGSGTDFTLTISSLQAEDVA |
| | | VYYCQQYYSTPLTFGQGTKVEIK (SEQ ID NO: 135) |
| GITR | VH: SEQ ID NO: 121 | VH: |
| | of U.S. Application | QVQLVQSGAEVKKPGASVKVSCKASGGTLSSYAISWVRQAPGQGL |
| | NO: US | EWMGWIDPNSGGTNYAQKFQGRVTMTRDTSTSTVYMELSSLRSED |
| | 2022/0106398 A1; | TAVYYCARQGLLWFGESGSIYYYYGMDVWGQGTTVTVSS (SEQ |
| | VL: SEQ ID NO: 125 | ID NO: 136) |
| | of U.S. Application | VL: |
| | NO: US | DIQMTQSPSSLSASVGDRVTITCRASRSISNYLNWYQQKPGKAPK |
| | 2022/0106398 A1 | LLIYGASSLQSGVPSRFSGSGSGTDFTLTISSLQPEDFATYYCQQ |
| | | SYSTLVTFGQGTKVEIK (SEQ ID NO: 137) |
| GITR | VH: SEQ ID NO: 129 | VH: |
| | of U.S. Application | QVQLVQSGAEVKKPGASVKVSCKASGYTFTSYGISWVRQAPGQGL |
| | NO: US | EWMGWMNPNSGNTGYAQKFQGRVTMTRDTSTSTVYMELSSLRSED |
| | 2022/0106398 A1; | TAVYYCARLGLWFGEYQYYFDYWGQGTLVTVSS (SEQ ID NO: |
| | VL: SEQ ID NO: 133 | 138) |
| | of U.S. Application | VL: |
| | NO: US | DIVMTQSPDSLAVSLGERATINCKSSQSVLYSSNNKNYLAWYQQK |
| | 2022/0106398 A1 | PGQPPKLLIYWASTREPGVPDRFSGSGSGTDFTLTISSLQAEDVA |
| | | VYYCQQYYSTPLTFGQGTKVEIK (SEQ ID NO: 139) |
| GITR | VH: SEQ ID NO: 137 | VH: |
| | of U.S. Application | EVQLLESGGGLVKPGGSLRLSCAASGFTFSSYWMSWVRQAPGKGL |
| | NO: US | EWVSGISWNGGTVGYADSVKGRFTISRDDSKNTLYLQMNSLKTED |
| | 2022/0106398 A1; | TAVYYCAKLGIAVKSHWYFDLWGRGTLVTVSS (SEQ ID NO: |
| | VL: SEQ ID NO: 141 | 140) |
| | of U.S. Application | VL: |
| | NO: US | DIQMTQSPSSLSASVGDRVTITCRASESISTWLAWYQQKPGKAPK |
| | 2022/0106398 A1 | LLIYAASSLQSGVPSRFSGSGSGTDFTLTISSLQPEDFATYYCQQ |
| | | SYTLPLTFGGGTKLEIK (SEQ ID NO: 141) |
| GITR | VH: SEQ ID NO: 145 | VH: |
| | of U.S. Application | QVQLVQSGAEVKKPGASVKVSCKASGYTFTSYDINWVRQAPGQGL |
| | NO: US | EWVGIINPSGGSTSYAQKFQGRVTMTRDTSTSTVYMELSSLRSED |
| | 2022/0106398 A1; | TAVYYCARASSGGYYYYYGMDVWGQGTTVTVSS (SEQ ID NO: |
| | VL: SEQ ID NO: 149 | 142) |
| | of U.S. Application | VL: |
| | NO: US | DIQMTQSPSSLSASVGDRVTITCRASQDIVNWLAWYQQKPGKAPK |
| | 2022/0106398 A1 | LLIYAASSLQSGVPSRFSGSGSGTDFTLTISSLQPEDFATYYCQQ |
| | | YYSYPLTFGQGTRLEIK (SEQ ID NO: 143) |
| GITR | VH: SEQ ID NO: 153 | VH: |
| | of U.S. Application | QVQLVQSGAEVKKPGASVKVSCKASGGTFSNYAISWVRQAPGQGL |
| | NO: US | EWMGVINPRGGSTTYAQKFQGRVTMTRDTSTSTVYMELSSLRSED |
| | 2022/0106398 A1; | TAVYYCARDYSIPYYGMDVWGQGTTVTVSS (SEQ ID NO: |
| | VL: SEQ ID NO: 157 | 144) |
| | of U.S. Application | VL: |
| | NO: US | DIQMTQSPSSLSASVGDRVTITCRASQSISTYLNWYQQKPGKAPK |
| | 2022/0106398 A1 | LLIYDASNLETGVPSRFSGSGSGTDFTLTISSLQPEDFATYYCQQ |
| | | SYSTPLTFGGGTKVEIK (SEQ ID NO: 145) |
| GITR | VH: SEQ ID NO: 161 | VH: |
| | of U.S. Application | EVQLLESGGGLVQPGGSLRLSCAASGFTFSNHYMSWVRQAPGKGL |
| | NO: US | EWVAVIALDGSYRYYADSVKGRFTISRDNSKNTLYLQMNSLRAED |
| | 2022/0106398 A1; | TAVYYCARVGPGGMDVRGQGTTVTVSS (SEQ ID NO: 146) |
| | VL: SEQ ID NO: 165 | VL: |
| | of U.S. Application | DIQMTQSPSSLSASVGDRVTITCQASQDISNYLNWYQQKPGKAPK |
| | NO: US | LLIYAASSLQSGVPSRFSGSGSGTDFTLTISSLQPEDFATYYCQQ |
| | 2022/0106398 A1 | SYNSPRVYTFGQGTKVEIK (SEQ ID NO: 147) |
| GITR | VH: SEQ ID NO: 169 | VH: |
| | of U.S. Application | QVQLVQSGAEVKKPGASVKVSCKASGGRFSTYALSWVRQAPGQGL |
| | NO: US | EWMGIINPTDGSTTYAQKFQGRVTMTRDTSTSTVYMELSSLRSED |
| | 2022/0106398 A1; | TAVYYCARDVYSSSWYSDYWGQGTLVTVSS (SEQ ID NO: |
| | VL: SEQ ID NO: 173 | 148) |
| | of U.S. Application | VL: |
| | NO: US | DIQMTQSPSSLSASVGDRVTITCRASQSISNWLAWYQQKPGKAPK |
| | 2022/0106398 A1 | LLIYAASTLQSGVPSRFSGSGSGTDFTLTISSLQPEDFATYYCQQ |
| | | SYSVPFTFGPGTKVDIK (SEQ ID NO: 149) |
| GITR | VH: SEQ ID NO: 177 | VH: |
| | of U.S. Application | EVQLLESGGGLVQPGGSLRLSCAASGFPFSTYAIHWVRQAPGKGL |
| | NO: US | EWVSAISGSGGSTYYADSVKGRFTISRDNSKNTLYLQMNSLRAED |
| | 2022/0106398 A1; | TAVYYCAGPDWYFDLWGRGTLVTVSS (SEQ ID NO: 150) |
| | VL: SEQ ID NO: 181 | VL: |
| | of U.S. Application | DIQMTQSPSSLSASVGDRVTITCRASQAISNSLAWYQQKPGKAPK |
| | NO: US | LLIYAASTLQRGVPSRFSGSGSGTDFTLTISSLQPEDFATYYCQQ |
| | 2022/0106398 A1 | YYSTPYTFGQGTKLEIK (SEQ ID NO: 151) |
| GITR | VH: SEQ ID NO: 185 | VH: |
| | of U.S. Application | QVQLVQSGAEVKKPGASVKVSCKASGGTFNNYAINWVRQAPGQGL |
| | NO: US | EWMGTINPRDGDTTYAQKFQGRVTMTRDTSTSTVYMELSSLRSED |
| | 2022/0106398 A1; | TAVYYCARVGYYGSGSYYSYYGMDVWGQGTTVTVSS (SEQ ID |
| | VL: SEQ ID NO: 189 | NO: 152) |
| | of U.S. Application | VL: |
| | NO: US | DIQMTQSPSSLSASVGDRVTITCRASQNITNWLAWYQQKPGKAPK |
| | 2022/0106398 A1 | LLIYAASSLQSGVPSRFSGSGSGTDFTLTISSLQPEDFATYYCQQ |
| | | AYSFPWTFGQGTKVEIK (SEQ ID NO: 153) |
| GITR | VH: SEQ ID NO: 203 | VH: |
| | of U.S. Application | QVQLVQSGAEVKKPGASVKVSCKASGGTFSSYAISWVRQAPGQGL |
| | NO: US | EWMGWVSGYNGNANYAQKFQGRVTMTRDTSTSTVYMELSSLRSED |
| | 2022/0106398 A1; | TAVYYCARDRVESGYSYHDAFDIWGQGTMVTVSS (SEQ ID |
| | VL: SEQ ID NO: 207 | NO: 154) |
| | of U.S. Application | VL: |
| | NO: US | DIQMTQSPSSLSASVGDRVTITCRTSQSIRRYLNWYQQKPGKAPK |
| | 2022/0106398 A1 | LLIYAASSLQSGVPSRFSGSGSGTDFTLTISSLQPEDFATYYCQQ |
| | | SYSIPWTFGPGTKVDIK (SEQ ID NO: 155) |
| GITR | VH: SEQ ID NO: 211 | VH: |
| | of U.S. Application | QVQLVQSGAEVKKPGASVKVSCKASGYTFTSYGISWVRQAPGQGL |
| | NO: US | EWMGWMNPNNGNTVYAQKFQGRVTMTRDTSTSTVYMELSSLRSED |
| | 2022/0106398 A1; | TAVYYCAKDSDWYGAFDIWGQGTMVTVSS (SEQ ID NO: |
| | VL: SEQ ID NO: 215 | 156) |
| | of U.S. Application | VL: |
| | NO: US | DIQMTQSPSSLSASVGDRVTITCQASQSISRWLAWYQQKPGKAPK |
| | 2022/0106398 A1 | LLIYSASSLQSGVPSRFSGSGSGTDFTLTISSLQPEDFATYYCQQ |
| | | SYSTPWTFGQGTKLEIK (SEQ ID NO: 157) |
| GITR | VH: SEQ ID NO: 219 | VH: |
| | of U.S. Application | QVQLVQSGAEVKKPGASVKVSCKASGYTFTSYDINWVRQAPGQGL |
| | NO: US | EWIGWMNTNSGDTGYAQKFQGRVTMTRDTSTSTVYMELSSLRSED |
| | 2022/0106398 A1; | TAVYYCARQAYSSSWYWYGWFDPWGQGTLVTVSS (SEQ ID |
| | VL: SEQ ID NO: 223 | NO: 158) |
| | of U.S. Application | VL: |
| | NO: US | DIQMTQSPSSLSASVGDRVTITCQASHDIDNYLNWYQQKPGKAPK |
| | 2022/0106398 A1 | LLIYAASNLQSGVPSRFSGSGSGTDFTLTISSLQPEDFATYYCQQ |
| | | ANSFPLTFGPGTKVDIK (SEQ ID NO: 159) |
| GITR | VH: SEQ ID NO: 227 | VH: |
| | of U.S. Application | QVQLVQSGAEVKKPGASVKVSCKASGYTFPNYGITWVRQAPGQGL |
| | NO: US | EWMGWMNPNSGYTGYAQNFQGRVTMTRDTSTSTVYMELSSLRSED |
| | 2022/0106398 A1; | TAVYYCARGMPGRGFDPWGQGTLVTVSS (SEQ ID NO: 160) |
| | VL: SEQ ID NO: 231 | VL: |
| | of U.S. Application | EIVMTQSPATLSVSPGERATLSCRASQSVSSNYLAWYQQKPGQAP |
| | NO: US | RLLIYGASTRATGIPARFSGSGSGTEFTLTISSLQSEDFAVYYCQ |
| | 2022/0106398 A1 | QYHTYPPTFGQGTKLEIK (SEQ ID NO: 161) |
| GITR | VH: SEQ ID NO: 235 | VH: |
| | of U.S. Application | QVQLVQSGAEVKKPGASVKVSCKASGYTFSSDYIHWVRQAPGQGL |
| | NO: US | EWMGRINPSGGSTLYARRFQGRVTMTRDTSTSTVYMELSSLRSED |
| | 2022/0106398 A1; | TAVYYCARERGAADTWYFDLWGRGTLVTVSS (SEQ ID NO: |
| | VL: SEQ ID NO: 239 | 162) |
| | of U.S. Application | VL: |
| | NO: US | DIQMTQSPSSLSASVGDRVTITCRASQSISSYLNWYQQKPGKAPK |
| | 2022/0106398 A1 | LLIYGASSLQSGVPSRFSGSGSGTDFTLTISSLQPEDFATYYCQQ |
| | | SYRTPLTFGGGTKVEIK (SEQ ID NO: 163) |
| GITR | VH: SEQ ID NO: 243 | VH: |
| | of U.S. Application | QVQLVQSGAEVKKPGASVKVSCKASGGTFSSYAISWVRQAPGQGL |
| | NO: US | EWMGIIDPTGGATAYAQEFQGRVTMTRDTSTSTVYMELSSLRSED |
| | 2022/0106398 A1; | TAVYYCARVGYYYGMDVWGQGTMVTVSS (SEQ ID NO: 164) |
| | VL: SEQ ID NO: 247 | VL: |
| | of U.S. Application | DIQMTQSPSSLSASVGDRVTITCRASQSVSTYLNWYQQKPGKAPK |
| | NO: US | LLIYAASSLQSGVPSRFSGSGSGTDFTLTISSLQPEDFATYYCQQ |
| | 2022/0106398 A1 | SYSTPLTFGGGTKVEIK (SEQ ID NO: 165) |
| GITR | VH: SEQ ID NO: 251 | VH: |
| | of U.S. Application | QVQLVQSGAEVKKPGSSVKVSCKASGGTFSSYAISWVRQAPGQGL |
| | NO: US | EWMGGIIPISSATSIPQKFQGRVTITADESTSTAYMELSSLRSED |
| | 2022/0106398 A1; | TAVYYCARSYDSRYYGMDVWGQGTTVTVSS (SEQ ID NO: |
| | VL: SEQ ID NO: 255 | 166) |
| | of U.S. Application | VL: |
| | NO: US | EIVMTQSPATLSVSPGERATLSCRASQTVGSRYLAWYQQKPGQAP |
| | 2022/0106398 A1 | RLLIYGASTRATGIPARFSGSGSGTEFTLTISSLQSEDFAVYYCQ |
| | | QYYSTPWTFGQGTRLEIK (SEQ ID NO: 167) |
| GITR | VH: SEQ ID NO: 259 | VH: |
| | of U.S. Application | QVQLVQSGAEVKKPGASVKVSCKASGGTFSSYAISWVRQAPGQGL |
| | NO: US | EWMGWMNPNSGDTGYAQKFQGRVTMTRDTSTSTVYMELSSLRSED |
| | 2022/0106398 A1; | TAVYYCARGPWYFDLWGRGTLVTVSS (SEQ ID NO: 168) |
| | VL: SEQ ID NO: 263 | VL: |
| | of U.S. Application | DIQMTQSPSSLSASVGDRVTITCRVSQGISNSLAWYQQKPGKAPK |
| | NO: US | LLIYAASSLQSGVPSRFSGSGSGTDFTLTISSLQPEDFATYYCQQ |
| | 2022/0106398 A1 | SYSTVYTFGQGTKLEIK (SEQ ID NO: 169) |
| GITR | VH: SEQ ID NO: 267 | VH: |
| | of U.S. Application | EVQLLESGGGLVKPGGSLRLSCAASGFMFSSYSMNWVRQAPGKGL |
| | NO: US | EWVSYISGNSGYTNYADSVKGRFTISRDDSKNTLYLQMNSLKTED |
| | 2022/0106398 A1; | TAVYYCARRLHGMDVWGQGTTVTVSS (SEQ ID NO: 170) |
| | VL: SEQ ID NO: 271 | VL: |
| | of U.S. Application | DIQMTQSPSSLSASVGDRVTITCRASQSISTWLAWYQQKPGKAPK |
| | NO: US | LLIYDASNLETGVPSRFSGSGSGTDFTLTISSLQPEDFATYYCQQ |
| | 2022/0106398 A1 | AYRFPVAFGGGTKVEIK (SEQ ID NO: 171) |
| GITR | VH: SEQ ID | VH: |
| | NO: 275 | EVQLLESGGGLVQPGGSLRLSCAASGFTFSSYWMHWVRQAPGKGL |
| | of U.S. Application | EWVSVISNSGGATYYADSVKGRFTISRDNSKNTLYLQMNSLRAED |
| | NO: US | TAVYYCAREGWGYGMDVWGQGTTVTVSS (SEQ ID NO: 172) |
| | 2022/0106398 A1; | VL: |
| | VL: SEQ ID NO: 279 | DIQMTQSPSSLSASVGDRVTITCRASQDIRNYLAWYQQKPGKAPK |
| | of U.S. Application | LLIYTASTLQSGVPSRFSGSGSGTDFTLTISSLQPEDFATYYCQQ |
| | NO: US | TFRTPLTFGGGTKVEIK (SEQ ID NO: 173) |
| | 2022/0106398 A1 | |
| GITR | VH: SEQ ID NO: 283 | VH: |
| | of U.S. Application | QVQLVQSGAEVKKPGASVKVSCKASGYTFTNYYMHWVRQAPGQGL |
| | NO: US | EWMGLITPSGGRTKYAQKFQGRVTMTRDTSTSTVYMELSSLRSED |
| | 2022/0106398 A1; | TAVYYCAREMEYSSSWYWFDPWGQGTLVTVSS (SEQ ID NO: |
| | VL: SEQ ID NO: 287 | 174) |
| | of U.S. Application | VL: |
| | NO: US | DIQMTQSPSSLSASVGDRVTITCQASQGISSYLNWYQQKPGKAPK |
| | 2022/0106398 A1 | LLIYAASTLQSGVPSRFSGSGSGTDFTLTISSLQPEDFATYYCQQ |
| | | SYSTPLTFGGGTKVEIK (SEQ ID NO: 175) |
| GITR | VH: SEQ ID NO: 291 | VH: |
| | of U.S. Application | QVQLVQSGAEVKKPGASVKVSCKASGYTFTRNYIHWVRQAPGQGL |
| | NO: US | EWMGWINPKSGGTKYAQKFQGRVTMTRDTSTSTVYMELSSLRSED |
| | 2022/0106398 A1; | TAVYYCARESGVVATEYWYFDLWGRGTLVTVSS (SEQ ID NO: |
| | VL: SEQ ID NO: 295 | 176) |
| | of U.S. Application | VL: |
| | NO: US | DIQMTQSPSSLSASVGDRVTITCRASQSISSYLNWYQQKPGKAPK |
| | 2022/0106398 A1 | LLIYAASSLQSGVPSRFSGSGSGTDFTLTISSLQPEDFATYYCQQ |
| | | SYSTPLTFGGGTKVEIK (SEQ ID NO: 177) |
| GITR | VH: SEQ ID NO: 299 | VH: |
| | of U.S. Application | QVQLVQSGAEVKKPGASVKVSCKASGYTFTSYYMHWVRQAPGQGL |
| | NO: US | EWMGWINPNSGGTNYAQKFQGRVTMTRDTSTSTVYMELSSLRSED |
| | 2022/0106398 A1; | TAVYYCASPGDYCSGGSCYSDDAFDIWGQGTMVTVSS (SEQ ID |
| | VL: SEQ ID NO: 303 | NO: 178) |
| | of U.S. Application | VL: |
| | NO: US | DIVMTQSPDSLAVSLGERATINCKSSQSIFYSSNSKNYLAWYQQK |
| | 2022/0106398 A1 | PGQPPKLLIYWASTRESGVPDRFSGSGSGTDFTLTISSLQAEDVA |
| | | VYYCQQYYSTPLTFGPGTKVDIK (SEQ ID NO: 179) |
| GITR | VH: SEQ ID NO: 307 | VH: |
| | of U.S. Application | QVQLVQSGAEVKKPGASVKVSCKASGGTFRNYAINWVRQAPGQGL |
| | NO: US | EWMGWMNPNSGNTGYAQKFQGRVTMTRDTSTSTVYMELSSLRSED |
| | 2022/0106398 A1; | TAVYYCAREDVDTASQAYFDYWGQGTLVTVSS (SEQ ID NO: |
| | VL: SEQ ID NO: 311 | 180) |
| | of U.S. Application | VL: |
| | NO: US | DIQMTQSPSSLSASVGDRVTITCRASQGISNYLAWYQQKPGKAPK |
| | 2022/0106398 A1 | LLIYSASNLQSGVPSRFSGSGSGTDFTLTISSLQPEDFATYYCQQ |
| | | GYSSRYTFGQGTKLEIK (SEQ ID NO: 181) |
| GITR | VH: SEQ ID NO: 315 | VH: |
| | of U.S. Application | EVQLLESGGGLVKPGGSLRLSCAASGFTFSSYWMSWVRQAPGKGL |
| | NO: US | EWVSSISWSSTYIYYADSVKGRFTISRDDSKNTLYLQMNSLKTED |
| | 2022/0106398 A1; | TAVYYCARDGQLGHWHFDLWGRGTLVTVSS (SEQ ID NO: |
| | VL: SEQ ID NO: 319 | 182) |
| | of U.S. Application | VL: |
| | NO: US | DIQMTQSPSSLSASVGDRVTITCRASQSISDWLAWYQQKPGKAPK |
| | 2022/0106398 A1 | LLIYEASKLATGVPSRFSGSGSGTDFTLTISSLQPEDFATYYCQQ |
| | | SYTTPLTFGGGTKVEIK (SEQ ID NO: 183) |
| GITR | VH: SEQ ID NO: 323 | VH: |
| | of U.S. Application | EVQLLESGGGLVQPGGSLRLSCAASGFTFRNHWMSWVRQAPGKGL |
| | NO: US | EWVSGISWNSGSIDYADSVKGRFTISRDNSKNTLYLQMNSLRAED |
| | 2022/0106398 A1; | TAVYYCAREEYATFDYWGQGTLVTVSS (SEQ ID NO: 184) |
| | VL: SEQ ID NO: 327 | VL: |
| | of U.S. Application | DIVMTQSPLSLPVTPGEPASISCRSSQSLLHSNGYNYLDWYLQKP |
| | NO: US | GQSPQLLIYLGSNRASGVPDRFSGSGSGTDFTLKISRVEAEDVGV |
| | 2022/0106398 A1 | YYCMQGTHWPPTFGPGTKVDIK (SEQ ID NO: 185) |
| GITR | VH: SEQ ID NO: 331 | VH: |
| | of U.S. Application | QVQLVQSGAEVKKPGASVKVSCKASGGSFSDYAVSWVRQAPGQGL |
| | NO: US | EWMGWMNPNSGNTGYAQKFQGRVTMTRDTSTSTVYMELSSLRSED |
| | 2022/0106398 A1; | TAVYYCARELVRDGYNFALDYWGQGTLVTVSS (SEQ ID NO: |
| | VL: SEQ ID NO: 335 | 186) |
| | of U.S. Application | VL: |
| | NO: US | DIQMTQSPSSLSASVGDRVTITCRASQDIRNDLAWYQQKPGKAPK |
| | 2022/0106398 A1 | LLIYAASSLQSGVPSRFSGSGSGTDFTLTISSLQPEDFATYYCQQ |
| | | SFSTFYTFGQGTKLEIK (SEQ ID NO: 187) |
| GITR | VH: SEQ ID NO: 339 | VH: |
| | of U.S. Application | QVQLVQSGAEVKKPGASVKVSCKASGYIFTNYWIQWVRQAPGQGL |
| | NO: US | EWMGWINPHSGATKYAERFQGRVTMTRDTSTSTVYMELSSLRSED |
| | 2022/0106398 A1; | TAVYYCARGLGSAFDIWGQGTMVTVSS (SEQ ID NO: 188) |
| | VL: SEQ ID NO: 343 | VL: |
| | of U.S. Application | DIVMTQSPLSLPVTPGEPASISCRSSQSLLHSNGYNYLDWYLQKP |
| | NO: US | GQSPQLLIYLGSNRASGVPDRFSGSGSGTDFTLKISRVEAEDVGV |
| | 2022/0106398 A1 | YYCMQALQTPLTFGGGTKVEIK (SEQ ID NO: 189) |
| GITR | VH: SEQ ID NO: 347 | VH: |
| | of U.S. Application | QVQLVQSGAEVKKPGASVKVSCKASGYTFTSHDIDWVRQAPGQGL |
| | NO: US | EWMGWMNPNNGNTVYAQRFQGRVTMTRDTSTSTVYMELSSLRSED |
| | 2022/0106398 A1; | TAVYYCARWKVYSGSYYGGAGYFDLWGRGTLVTVSS (SEQ ID |
| | VL: SEQ ID NO: 351 | NO: 190) |
| | of U.S. Application | VL: |
| | NO: US | DIQMTQSPSSLSASVGDRVTITCRASQSISSYLNWYQQKPGKAPK |
| | 2022/0106398 A1 | LLIYRASHLEGGVPSRFSGSGSGTDFTLTISSLQPEDFATYYCQQ |
| | | ADSLPLTFGQGTKVEIK (SEQ ID NO: 191) |
| GITR | VH: SEQ ID NO: 355 | VH: |
| | of U.S. Application | QVQLVQSGAEVKKPGASVKVSCKASGYTFTSHDIDWVRQAPGQGL |
| | NO: US | EWMGWINPSGDSTSYAQKFQGRVTMTRDTSTSTVYMELSSLRSED |
| | 2022/0106398 A1; | TAVYYCARYYGGNSYAFDIWGQGTMVTVSS (SEQ ID NO: |
| | VL: SEQ ID NO: 359 | 192) |
| | of U.S. Application | VL: |
| | NO: US | DIQMTQSPSSLSASVGDRVTITCRASESISPWVAWYQQKPGKAPK |
| | 2022/0106398 A1 | LLIYAASTLQSGVPSRFSGSGSGTDFTLTISSLQPEDFATYYCQQ |
| | | GTSTPYTFGQGTKLEIK (SEQ ID NO: 193) |
| GITR | VH: SEQ ID NO: 363 | VH: |
| | of U.S. Application | QVQLVQSGAEVKKPGASVKVSCKASGYTFTSYGISWVRQAPGQGL |
| | NO: US | EWMGWMNPNSGNTGYAQKFQGRVTMTRDTSTSTVYMELSSLRSED |
| | 2022/0106398 A1; | TAVYYCAREVYGDLDYWGQGTLVTVSS (SEQ ID NO: 194) |
| | VL: SEQ ID NO: 367 | VL: |
| | of U.S. Application | DIQMTQSPSSLSASVGDRVTITCRASQGIDNWLAWYQQKPGKAPK |
| | NO: US | LLIYGASTLQSGVPSRFSGSGSGTDFTLTISSLQPEDFATYYCQQ |
| | 2022/0106398 A1 | SYTARFTFGPGTKVDIK (SEQ ID NO: 195) |
| GITR | VH: SEQ ID NO: 371 | VH: |
| | of U.S. Application | QVQLVQSGAEVKKPGASVKVSCKASGGSFSNYAINWVRQAPGQGL |
| | NO: US | EWMGWMNPYSGNTGYAQKFQGRVTMTRDTSTSTVYMELSSLRSED |
| | 2022/0106398 A1; | TAVYYCAREITANYYYGMDVWGQGTKVTVSS (SEQ ID NO: |
| | VL: SEQ ID NO: 375 | 196) |
| | of U.S. Application | VL: |
| | NO: US | DIQMTQSPSSLSASVGDRVTITCQASQDISNYLNWYQQKPGKAPK |
| | 2022/0106398 A1 | LLIYEASVLESGVPSRFSGSGSGTDFTLTISSLQPEDFATYYCQQ |
| | | SYTTQYTFGQGTKVEIK (SEQ ID NO: 197) |
| GITR | VH: SEQ ID NO: 379 | VH: |
| | of U.S. Application | QVQLVQSGAEVKKPGSSVKVSCKASGGTFSTYAINWVRQAPGQGL |
| | NO: US | EWMGIINPTDGDTSYAQKFQGRVTITADESTSTAYMELSSLRSED |
| | 2022/0106398 A1; | TAVYYCARAAYYYYGMDVWGQGTTVTVSS (SEQ ID NO: |
| | VL: SEQ ID NO: 383 | 198) |
| | of U.S. Application | VL: |
| | NO: US | DIVMTQSPLSLPVTPGEPASISCRSSQSLLHSNGYNYLDWYLQKP |
| | 2022/0106398 A1 | GQSPQLLIYLGSNRASGVPDRFSGSGSGTDFTLKISRVEAEDVGV |
| | | YYCMQALHIPYTFGQGTKLEIK (SEQ ID NO: 199) |
| GITR | VH: SEQ ID NO: 387 | VH: |
| | of U.S. Application | QVQLVQSGAEVKKPGASVKVSCKASGYSFTSHDIDWVRQAPGQGL |
| | NO: US | EWMGWINPNSGGTNYAQKFQGRVTMTRDTSTSTVYMELSSLRSED |
| | 2022/0106398 A1; | TAVYYCARIRGYYGSGSYHDAFDIWGQGTTVTVSS (SEQ ID |
| | VL: SEQ ID NO: 391 | NO: 200) |
| | of U.S. Application | VL: |
| | NO: US | DIQMTQSPSSLSASVGDRVTITCRASQTISTYLNWYQQKPGKAPK |
| | 2022/0106398 A1 | LLIYAASSLQSGVPSRFSGSGSGTDFTLTISSLQPEDFATYYCQQ |
| | | SYSTPWTFGQGTKVEIK (SEQ ID NO: 201) |
| 4-1BB | VH: SEQ ID NO: 17 | VH: |
| | of PCT Application | EVQLVQSGAEVKKPGESLRISCKGSGYSFSTYWISWVRQMPGKGL |
| | NO: WO | EWMGKIYPGDSYTNYSPSFQGQVTISADKSISTAYLQWSSLKASD |
| | 2017/130076 A1; | TAMYYCARGYGIFDYWGQGTLVTVSS (SEQ ID NO: 202) |
| | VL: SEQ ID NO: 18 | VL: |
| | of PCT Application | SYELTQPPSVSVSPGQTASITCSGDNIGDQYAHWYQQKPGQSPVL |
| | NO: WO | VIYQDKNRPSGIPERFSGSNSGNTATLTISGTQAMDEADYYCATY |
| | 2017/130076 A1 | TGFGSLAVFGGGTKLTVL (SEQ ID NO: 203) |
| 4-1BB | MOR-6032 | VH: |
| | VH: SEQ ID NO: 4 of | QVQLVQSGAEVKKPGSSVKVSCKASGGTFNSYAISWVRQAPGQGL |
| | U.S. Pat. No. | EWMGGIIPGFGTANYAQKFQGRVTITADESTSTAYMELSSLRSED |
| | 10,640,568; VL: | TAVYYCARKNEEDGGFDHWGQGTLVTVSS (SEQ ID NO: |
| | SEQ ID NO: 9 of | 204) |
| | U.S. Pat. No. | VL: |
| | 10,640,568 | DIELTQPPSVSVAPGQTARISCSGDNLGDYYASWYQQKPGQAPVL |
| | | VIYDDSNRPSGIPERFSGSNSGNTATLTISGTQAEDEADYYCQTW |
| | | DGTLHFVFGGGTKLTVL (SEQ ID NO: 205) |
| 4-1BB | MOR-7361 | VH: |
| | VH: SEQ ID NO: 18 | QVQLVESGGGLVQPGGSLRLSCAASGFTFSDYYMHWVRQAPGKGL |
| | of U.S. Pat. No. | EWVSVISGSGSNTYYADSVKGRFTISRDNSKNTLYLQMNSLRAED |
| | 10,640,568; VL: | TAVYYCARLYAQFEGDFWGQGTLVTVSS (SEQ ID NO: 206) |
| | SEQ ID NO: 23 of | VL: |
| | U.S. Pat. No. | DIELTQPPSVSVAPGQTARISCSGDNIGSKYVSWYQQKPGQAPVL |
| | 10,640,568 | VIYSDSERPSGIPERFSGSNSGNTATLTISGTQAEDEADYYCQSW |
| | | DGSISRVFGGGTKLTVL (SEQ ID NO: 207) |
| 4-1BB | MOR-7480 | VH: |
| | VH: SEQ ID NO: 32 | QVQLVQSGAEVKKPGESLKISCKGSGYSFSTYWISWVRQMPGKGL |
| | of U.S. Pat. No. | EWMGKIYPGDSYTNYSPSFQGQVTISADKSISTAYLQWSSLKASD |
| | 10,640,568; VL: | TAMYYCARGYGIFDYWGQGTLVTVSS (SEQ ID NO: 208) |
| | SEQ ID NO: 37 of | VL: |
| | U.S. Pat. No. | DIELTQPPSVSVAPGQTARISCSGDNIGDQYAHWYQQKPGQAPVV |
| | 10,640,568 | VIYQDKNRPSGIPERFSGSNSGNTATLTISGTQAEDEADYYCATY |
| | | TGFGSLAVFGGGTKLTVL (SEQ ID NO: 209) |
| 4-1BB | MOR-7483 | VH: |
| | VH: SEQ ID NO: 32 | QVQLVQSGAEVKKPGESLKISCKGSGYSFSTYWISWVRQMPGKGL |
| | of U.S. Pat. No. | EWMGKIYPGDSYTNYSPSFQGQVTISADKSISTAYLQWSSLKASD |
| | 10,640,568; VL: | TAMYYCARGYGIFDYWGQGTLVTVSS (SEQ ID NO: 208) |
| | SEQ ID NO: 56 of | VL: |
| | U.S. Pat. No. | DIELTQPPSVSVAPGQTARISCSGDNIGDQYAHWYQQKPGQAPVV |
| | 10,640,568 | VIYQDKNRPSGIPERFSGSNSGNTATLTISGTQAEDEADYYCSTY |
| | | TFVGFTTVFGGGTKLTVL (SEQ ID NO: 210) |
| 4-1BB | VH: SEQ ID NO: 11 | VH: |
| | of PCT Application | QVQLQQSGAEVKKPGASVKLSCKASGYTFSSYWMHWVRQAPGQGL |
| | NO: WO | EWIGEINPGNGHTNYNEKFKSRATMTRDTSTSTAYMELSSLRSED |
| | 2018/127787 A1; | SAVYYCARSFTTARAFAYWGQGTLVTVSS (SEQ ID NO: |
| | VL: SEQ ID NO: 10 | 211) |
| | of PCT Application | VL: |
| | NO: WO | DIVMTQSPAFLSVTPGEKVTITCRASQTISDYLHWYQQKPDQAPK |
| | 2018/127787 A1 | LLIKYASQSISGIPSRFSGSGSGTDFTFTISSLEAEDAATYYCQD |
| | | GHSWPPTFGQGTKLEIK (SEQ ID NO: 212) |
| 4-1BB | VH: SEQ ID NO: 12 | VH: |
| | of PCT Application | QVQLQQSGAEVKKPGASVKLSCKASGYTFSSYWMHWVRQAPGQGL |
| | NO: WO | EWIGEINPGNGHTNYNEKFKSRATMTRDTSTSTAYMELSSLRSED |
| | 2018/127787 A1; | SAVYYCARSFKTARAFAYWGQGTLVTVSS (SEQ ID NO: |
| | VL: SEQ ID NO: 10 | 213) |
| | of PCT Application | VL: |
| | NO: WO | DIVMTQSPAFLSVTPGEKVTITCRASQTISDYLHWYQQKPDQAPK |
| | 2018/127787 A1 | LLIKYASQSISGIPSRFSGSGSGTDFTFTISSLEAEDAATYYCQD |
| | | GHSWPPTFGQGTKLEIK (SEQ ID NO: 212) |
| 4-1BB | VH: SEQ ID NO: 13 | VH: |
| | of PCT Application | QVQLVQSGAEVKKPGASVKLSCKASGYTFSSYWMHWVRQAPGQGL |
| | NO: WO | EWIGEINPGNGHTNYNEKFKSRVTMTRDTSTSTAYMELSSLRSED |
| | 2018/127787 A1; | SAVYYCARSFKTARAFAYWGQGTLVTVSS (SEQ ID NO: |
| | VL: SEQ ID NO: 10 | 214) |
| | of PCT Application | VL: |
| | NO: WO | DIVMTQSPAFLSVTPGEKVTITCRASQTISDYLHWYQQKPDQAPK |
| | 2018/127787 A1 | LLIKYASQSISGIPSRFSGSGSGTDFTFTISSLEAEDAATYYCQD |
| | | GHSWPPTFGQGTKLEIK (SEQ ID NO: 212) |
| 4-1BB | VH: SEQ ID NO: 14 | VH: |
| | of PCT Application | QVQLVQSGAEVKKPGASVKLSCKASGYTFSSYWMHWVRQAPGQGL |
| | NO: WO | EWIGEINPGNGHTNYNEKFKSRVTMTRDTSTSTAYMELSSLRSED |
| | 2018/127787 A1; | SAVYYCARSFKTARAFAYWGQGTLVTVSS (SEQ ID NO: |
| | VL: SEQ ID NO: 10 | 214) |
| | of PCT Application | VL: |
| | NO: WO | DIVMTQSPAFLSVTPGEKVTITCRASQTISDYLHWYQQKPDQAPK |
| | 2018/127787 A1 | LLIKYASQSISGIPSRFSGSGSGTDFTFTISSLEAEDAATYYCQD |
| | | GHSWPPTFGQGTKLEIK (SEQ ID NO: 212) |
| CD27 | hCD27.15 | VH: |
| | VH: SEQ ID NO: 8 | EVRLQQSGADLVKPGASVKLSCTASGFIIKATYMHWVRQRPEQGL |
| | of U.S. Application | EWIGRIDPANGETKYDPKFQVKATITADTSSSTAYLQLNSLTSDD |
| | NO: US | TAVYYCARYAWYFDVWGAGTTVTVSS (SEQ ID NO: 215) |
| | 2019/0135933 A1; | VL: |
| | VL: SEQ ID NO: 10 | DIQMTQSPASLSASVGDTVTITCRASENIYSFLAWYHQKQGRSPQ |
| | of U.S. Application | LLVYHAKTLAEGVPSRFSGSGSGTQFSLKINSLQAEDFGSYYCQH |
| | NO: US | YYGSPLTFGAGTKLEVK (SEQ ID NO: 216) |
| | 2019/0135933 A1 | |
| CD27 | 1F5 | VH: |
| | VH: SEQ ID NO: 17 | QVQLVESGGGVVQPGRSLRLSCAASGFTFSSYDMHWVRQAPGKGL |
| | of U.S. Application | EWVAVIWYDGSNKYYADSVKGRFTISRDNSKNTLYLQMNSLRAED |
| | NO: US | TAVYYCARGSGNWGFFDYWGQGTLVTVSS (SEQ ID NO: |
| | 2019/0135933 A1; | 217) |
| | VL: SEQ ID NO: 18 | VL: |
| | of U.S. Application | DIQMTQSPSSLSASVGDRVTITCRASQGISRWLAWYQQKPEKAPK |
| | NO: US | SLIYAASSLQSGVPSRFSGSGSGTDFTLTISSLQPEDFATYYCQQ |
| | 2019/0135933 A1 | YNTYPRTFGQGTKVEIK (SEQ ID NO: 218) |
| CD27 | VH: SEQ ID NO: 7 of | VH: |
| | PCT Application NO: | QIQLVQSGPELKKPGETVKISCKASGYTFTNYGMNWVKQAPGKGL |
| | WO 2018/058022 | KWMGWINTNTGEPTYAEEFKGRFAFSLETSATTAYLQINNLKNED |
| | A1; VL: SEQ ID | TATYFCAREGDAMDYWGQGTSVTVSS (SEQ ID NO: 219) |
| | NO: 8 of PCT | VL: |
| | Application NO: WO | QIVLTQSPAIMSASPGEKVTMTCSASSSVSYMHWYQQKSGTSPKR |
| | 2018/058022 A1 | WIYDTSKLASGVPARFSGSGSGTSYSLTISSMEAEDAATYYCQQW |
| | | NSYPFTFGSGTKLEIK (SEQ ID NO: 220) |
| CD27 | VH: SEQ ID NO: 9 of | VH: |
| | PCT Application NO: | EIQLVQSGAEVKKPGASVKVSCKASGYTFTNYGMNWVKQAPGQGL |
| | WO 2018/058022 | KWMGXIXXXXGEPTYAEEFKGRFTFTLDTSISTAYMELSSLRSED |
| | A1; VL: SEQ ID | TAVYYCAREGXXXDYWGQGTTVTVSS (SEQ ID NO: 221) |
| | NO: 14 of PCT | VL: |
| | Application NO: WO | EIVLTQSPATLSLSPGERATLSCSASSSVSYMHWYQQKPGQAPKR |
| | 2018/058022 A1 | XIYXXSKLASGVPARFSGSGSGTDYSLTISSLEPEDFAVYYCQQX |
| | | XXYPFTFGQGTKLEIK (SEQ ID NO: 222) |
| CD27 | VH: SEQ ID NO: 32 | VH: |
| | of PCT Application | EIQLVQSGAEVKKPGASVKVSCKASGYTFTNYGMNWVKQAPGQGL |
| | NO: WO | KWMGXIXXXXGEPTYAEEFKGRFAFTLDTSISTAYMELSSLRSED |
| | 2018/058022 A1; | TAVYYCAREGXXXDYWGQGTTVTVSS (SEQ ID NO: 223) |
| | VL: SEQ ID NO: 33 | VL: |
| | of PCT Application | EIVLTQSPATLSLSPGEKATLSCSASSSVSYMHWYQQKPGQAPKR |
| | NO: WO | XIYXXSKLASGVPARFSGSGSGTDYSLTISSLEAEDFAVYYCQQX |
| | 2018/058022 A1 | XXYPFTFGQGTKLEIK (SEQ ID NO: 224) |
| CD27 | VH: SEQ ID NO: 34 | VH: |
| | of PCT Application | EIQLVQSGAEVKKPGASVKVSCKASGYTFTNYGMNWVKQAPGQGL |
| | NO: WO | KWMGWINTNTGEPTYAEEFKGRFAFTLDTSISTAYMELSSLRSED |
| | 2018/058022 A1; | TAVYYCAREGDAMDYWGQGTTVTVSS (SEQ ID NO: 225) |
| | VL: SEQ ID NO: 35 | VL |
| | of PCT Application | EIVLTQSPATLSLSPGEKATLSCSASSSVSYMHWYQQKPGQAPKR |
| | NO: WO | WIYDTSKLASGVPARFSGSGSGTDYSLTISSLEAEDFAVYYCQQW |
| | 2018/058022 A1 | NSYPFTFGQGTKLEIK (SEQ ID NO: 226) |
| CD27 | VH: SEQ ID NO: 39 | VH: |
| | of PCT Application | EIQLVQSGAEVKKPGASVKVSCKASGYTFTNYGMNWVKQAPGQGL |
| | NO: WO | KWMGWINTNTGEPTYAEEFKGRFTFTLDTSISTAYMELSSLRSED |
| | 2018/058022 A1; | TAVYYCAREGDAMDYWGQGTTVTVSS (SEQ ID NO: 227) |
| | VL: SEQ ID NO: 40 | VL: |
| | of PCT Application | EIVLTQSPATLSLSPGERATLSCSASSSVSYMHWYQQKPGQAPKR |
| | NO: WO | WIYDTSKLASGVPARFSGSGSGTDYSLTISSLEPEDFAVYYCQQW |
| | 2018/058022 A1 | NSYPFTFGQGTKLEIK (SEQ ID NO: 228) |
| CD27 | VH: SEQ ID NO: 10 | VH: |
| | of PCT Application | EIQLVQSGAEVKKPGASVKVSCKASGYTFTNYGMNWVKQAPGQGL |
| | NO: WO | KWMGWINTNTGEPTYAEEFKGRFTFTLDTSISTAYMELSSLRSED |
| | 2018/058022 A1; | TAVYYCAREGDAMDYWGQGTTVTVSS (SEQ ID NO: 227) |
| | VL: SEQ ID NO: 15 | VL: |
| | of PCT Application | EIVLTQSPATLSLSPGERATLSCSASSSVSYMHWYQQKPGQAPKR |
| | NO: WO | WIYDTSKLASGVPARFSGSGSGTDYSLTISSLEPEDFAVYYCQQW |
| | 2018/058022 A1 | NSYPFTFGQGTKLEIK (SEQ ID NO: 228) |
| CD30 | C10 | VH: |
| | VH: SEQ ID NO: 2 of | QIQLQQSGPEVVKPGASVKISCKASGYTFTDYYITWVKQKPGQGL |
| | U.S. Pat. No. | EWIGWIYPGSGNTKYNEKFKGKATLTVDTSSSTAFMQLSSLTSED |
| | 9,574,006; VL: SEQ | TAVYFCANYGNYWFAYWGQGTQVTVSA (SEQ ID NO: 229) |
| | ID NO: 1 of U.S. | VL: |
| | Pat. No. | DIVLTQSPASLAVSLGQRATISCKASQSVDFDGDSYMNWYQQKPG |
| | 9,574,006 | QPPKVLIYAASNLESGIPARFSGSGSGTDFTLNIHPVEEEDAATY |
| | | YCQQSNEDPWTFGGGTKLEIK (SEQ ID NO: 230) |
| CD30 | VH: SEQ ID NO: 11 | VH: |
| | of U.S. Pat. No. | QVQLVQSGAEVKKPGASVKVSCKVSGYTFTDYYITWVRQAPGQAL |
| | 9,574,006; VL: SEQ | EWMGWIYPGSGNTKYSQKFQGRFVFSVDTSASTAYLQISSLKAED |
| | ID NO: 10 of U.S. | TAVYYCANYGNYWFAYWGQGTLVTVSS (SEQ ID NO: 231) |
| | Pat. No. | VL: |
| | 9,574,006 | EIVLTQSPDSLAVSLGERATINCKASQSVDFDGDSYLNWYQQKPG |
| | | QPPKVLIYAASTLQSGVPSRFSGSGSGTDFTLTINSLEAEDAATY |
| | | YCQQSNEDPWTFGGGTKVEIK (SEQ ID NO: 232) |
| CD30 | VH: SEQ ID NO: 4 of | VH: |
| | U.S. Pat. No. | QIQLVQSGPEVKKPGASVKVSCKASGYTFTDYYITWVRQAPGQGL |
| | 9,574,006; VL: SEQ | EWMGWIYPGSGNTKYNEKFQGRFVFSVDTSASTAYLQISSLKAED |
| | ID NO: 3 of U.S. | TAVYFCANYGNYWFAYWGQGTLVTVSS (SEQ ID NO: 233) |
| | Pat. No. | VL: |
| | 9,574,006 | DIVLTQSPDSLAVSLGERATINCKASQSVDFDGDSYMNWYQQKPG |
| | | QPPKVLIYAASNLESGIPARFSGSGSGTDFTLTINSLEAEDAATY |
| | | YCQQSNEDPWTFGGGTKVEIK (SEQ ID NO: 234) |
| CD30 | VH: SEQ ID NO: 9 of | VH: |
| | U.S. Pat. No. | QLQLVQSGAEVKKPGASVKVSCKVSGYTFTSYYISWVRQAPGQAL |
| | 9,574,006; VL: SEQ | EWMGWIYAGSGNTKYSQKFQGRFVFSVDTSASTAYLQISSLKAED |
| | ID NO: 5 of U.S. | TAVYYCANYGNYWFAYWGQGTLVTVSS (SEQ ID NO: 235) |
| | Pat. No. | VL: |
| | 9,574,006 | EIVLTQSPDSLAVSLGERATINCKASQSVDFDGDSYLNWYQQKPG |
| | | QPPKVLIYAASTLQSGVPSRFSGSGSGTDFTLTINSLEAEDAATY |
| | | YCQQSNEDPWTFGGGTKVEIK (SEQ ID NO: 232) |
| CD30 | VH: SEQ ID NO: 9 of | VH: |
| | U.S. Pat. No. | QLQLVQSGAEVKKPGASVKVSCKVSGYTFTSYYISWVRQAPGQAL |
| | 9,574,006; VL: SEQ | EWMGWIYAGSGNTKYSQKFQGRFVFSVDTSASTAYLQISSLKAED |
| | ID NO: 6 of U.S. | TAVYYCANYGNYWFAYWGQGTLVTVSS (SEQ ID NO: 235) |
| | Pat. No. | VL: |
| | 9,574,006 | AIVLTQSPDSLAVSLGERATINCKASQSVDFDGDSYLNWYQQKPG |
| | | QPPKVLIYAASTLETGVPSRFSGSGSGTDFTLTINSLEAEDAATY |
| | | YCQQSNEDPWTFGGGTKVEIK (SEQ ID NO: 236) |
| CD30 | VH: SEQ ID NO: 7 of | VH: |
| | U.S. Pat. No. | QLQLVQSGPEVKKPGASVKVSCKVSGYTFTDYYITWVRQAPGQAL |
| | 9,574,006; VL: SEQ | EWMGWIYPGSGNTKYNEKFQGRFVFSVDTSASTAYLQISSLKAED |
| | ID NO: 5 of U.S. | TAVYFCANYGNYWFAYWGQGTLVTVSS (SEQ ID NO: 237) |
| | Pat. No. | VL |
| | 9,574,006 | EIVLTQSPDSLAVSLGERATINCKASQSVDFDGDSYLNWYQQKPG |
| | | QPPKVLIYAASTLQSGVPSRFSGSGSGTDFTLTINSLEAEDAATY |
| | | YCQQSNEDPWTFGGGTKVEIK (SEQ ID NO: 232) |
| CD30 | VH: SEQ ID NO: 7 of | VH: |
| | U.S. Pat. No. | QLQLVQSGPEVKKPGASVKVSCKVSGYTFTDYYITWVRQAPGQAL |
| | 9,574,006; VL: SEQ | EWMGWIYPGSGNTKYNEKFQGRFVFSVDTSASTAYLQISSLKAED |
| | ID NO: 6 of U.S. | TAVYFCANYGNYWFAYWGQGTLVTVSS (SEQ ID NO: 237) |
| | Pat. NO. | VL: |
| | 9,574,006 | AIVLTQSPDSLAVSLGERATINCKASQSVDFDGDSYLNWYQQKPG |
| | | QPPKVLIYAASTLETGVPSRFSGSGSGTDFTLTINSLEAEDAATY |
| | | YCQQSNEDPWTFGGGTKVEIK (SEQ ID NO: 236) |
| CD30 | VH: SEQ ID NO: 8 of | VH: |
| | U.S. Pat. No. | QLQLVQSGAEVKKPGASVKVSCKVSGYTFTDYYITWVRQAPGQAL |
| | 9,574,006; VL: SEQ | EWMGWIYPGSGNTKYSQKFQGRFVFSVDTSASTAYLQISSLKAED |
| | ID NO: 5 of U.S. | TAVYYCANYGNYWFAYWGQGTLVTVSS (SEQ ID NO: 238) |
| | Pat. No. | VL: |
| | 9,574,006 | EIVLTQSPDSLAVSLGERATINCKASQSVDFDGDSYLNWYQQKPG |
| | | QPPKVLIYAASTLQSGVPSRFSGSGSGTDFTLTINSLEAEDAATY |
| | | YCQQSNEDPWTFGGGTKVEIK (SEQ ID NO: 232) |
| CD30 | VH: SEQ ID NO: 8 of | VH: |
| | U.S. Pat. No. | QLQLVQSGAEVKKPGASVKVSCKVSGYTFTDYYITWVRQAPGQAL |
| | 9,574,006; VL: SEQ | EWMGWIYPGSGNTKYSQKFQGRFVFSVDTSASTAYLQISSLKAED |
| | ID NO: 6 of U.S. | TAVYYCANYGNYWFAYWGQGTLVTVSS (SEQ ID NO: 238) |
| | Pat. No. | VL: |
| | 9,574,006 | AIVLTQSPDSLAVSLGERATINCKASQSVDFDGDSYLNWYQQKPG |
| | | QPPKVLIYAASTLETGVPSRFSGSGSGTDFTLTINSLEAEDAATY |
| | | YCQQSNEDPWTFGGGTKVEIK (SEQ ID NO: 236) |
| EpoR | VH: SEQ ID NO: 7 of | VH: |
| | PCT Application NO: | MAQVQLVESGAEVKKPGASVKVSCKASGYTFTGYYMHWVRQAPGQ |
| | WO 2014/035693 | GLEWMGWINPNSGGTNYAQKFQGRVTMTRDTSISTAYMELSRLRS |
| | A2; VL: SEQ ID | DDTAVYYCARLSSGWTFDYWGQGTLVTVSS (SEQ ID NO: |
| | NO: 8 of PCT | 239) |
| | Application NO: WO | VL: |
| | 2014/035693 A2 | EIVLTQSPDSLAVSLGERATINCKSSQSVLYSPNKNYLAWYQQKP |
| | | GQPPKLLIYWASTRESGVPERFSGSGSGTDFTLTISSLHAEDVAL |
| | | YYCQQSYSLPFTFGPGTKVEIKR (SEQ ID NO: 240) |
| EpoR | VH: SEQ ID NO: 21 | VH: |
| | of PCT Application | MAQVQLVQSGAEVKKPGSSVKVSCKASGGTFSSYAISWVRQAPGQ |
| | NO: WO | GLEWMGGIIPIFGTANYAQKFQGRVTITADESTSTAYMELSSLRS |
| | 2014/035693 A2; | EDTAVYYCARDQGYYYGSGGLDYWGQGTLVTVSS (SEQ ID |
| | VL: SEQ ID NO: 22 | NO: 241) |
| | of PCT Application | VL: |
| | NO: WO | DIQMTQSPSSLSASVGDRVTITCRASQSISSYLNWYQQKPGKAPK |
| | 2014/035693 A2 | LLIYAASSLQSGVPSRFSGSGSGTDFTLTISSLQPEDFATYYCLQ |
| | | DYNYPLTFGGGTKVEIK (SEQ ID NO: 242) |
| EpoR | VH: SEQ ID NO: 23 | VH: |
| | of PCT Application | DIQMTQSPSSLSASVGDRVTITCRASQSISSYLNWYQQKPGKAPK |
| | NO: WO | LLIYAASSLQSGVPSRFSGSGSGTDFTLTISSLQPEDFATYYCLQ |
| | 2014/035693 A2; | DYNYPLTFGGGTKVEIK (SEQ ID NO: 242) |
| | VL: SEQ ID NO: 24 | VL: |
| | of PCT Application | QSALTQPASVSGSPGQSITISCTGTSSDVGAYNYVSWYQQHPGKA |
| | NO: WO | PKLMIYEVTKRPSGVPDRFSGSKSGNTASLTISGLQAEDEADYYC |
| | 2014/035693 A2 | ISFTASSTWAFGGGTKLTVLG (SEQ ID NO: 243) |
| EpoR | VH: SEQ ID NO: 15 | VH: |
| | of U.S. Application | QVQLQESGPGLVKPSETLSLTCTVSGASISSYYWSWIRQPPGKGL |
| | NO: US | EWIGYIXXXGSTNYNPSLKSRVTISVDTSKNQFSLKLRSVTAADT |
| | 2005/0227289 A1; | AVYYCARERLGIGDYWGQGTLVTVSS (SEQ ID NO: 244) |
| | VL: SEQ ID NO: 17 | VL: |
| | of U.S. Application | DIQLTQSPSSLSASVGDRVTITCRASQGIRNDLGWYQQKPGKAPK |
| | NO: US | RLIYAASSLQSGVPSRFSGSGSGTEFTLTISSLQPEDFATYYCLQ |
| | 2005/0227289 A1 | HNTYPPTFGQGTKVEIK (SEQ ID NO: 245) |
| EpoR | VH: SEQ ID NO: 7 of | VH: |
| | U.S. Application NO: | QVQLQESGPGLVKPSETLSLTCTVSGASISSYYWSWIRQPPGKGL |
| | US 2005/0227289 | EWIGYIGGEGSTNYNPSLKSRVTISVDTSKNQFSLKLRSVTAADT |
| | A1; VL: SEQ ID | AVYYCARERLGIGDYWGQGTLVTVSS (SEQ ID NO: 246) |
| | NO: 17 of U.S. | VL: |
| | Application NO: US | DIQLTQSPSSLSASVGDRVTITCRASQGIRNDLGWYQQKPGKAPK |
| | 2005/0227289 A1 | RLIYAASSLQSGVPSRFSGSGSGTEFTLTISSLQPEDFATYYCLQ |
| | | HNTYPPTFGQGTKVEIK (SEQ ID NO: 245) |
| EpoR | VH: SEQ ID NO: 8 of | VH: |
| | U.S. Application NO: | QVQLQESGPGLVKPSETLSLTCTVSGASISSYYWSWIRQPPGKGL |
| | US 2005/0227289 | EWIGYIAGTGSTNYNPSLKSRVTISVDTSKNQFSLKLRSVTAADT |
| | A1; VL: SEQ ID | AVYYCARERLGIGDYWGQGTLVTVSS (SEQ ID NO: 247) |
| | NO: 17 of U.S. | VL: |
| | Application NO: US | DIQLTQSPSSLSASVGDRVTITCRASQGIRNDLGWYQQKPGKAPK |
| | 2005/0227289 A1 | RLIYAASSLQSGVPSRFSGSGSGTEFTLTISSLQPEDFATYYCLQ |
| | | HNTYPPTFGQGTKVEIK (SEQ ID NO: 245) |
| EpoR | VH: SEQ ID NO: 9 of | VH: |
| | U.S. Application NO: | QVQLQESGPGLVKPSETLSLTCTVSGASISSYYWSWIRQPPGKGL |
| | US 2005/0227289 | EWIGYIGYSGSTNYNPSLKSRVTISVDTSKNQFSLKLRSVTAADT |
| | A1; VL: SEQ ID | AVYYCARERLGIGDYWGQGTLVTVSS (SEQ ID NO: 248) |
| | NO: 17 of U.S. | VL: |
| | Application NO: US | DIQLTQSPSSLSASVGDRVTITCRASQGIRNDLGWYQQKPGKAPK |
| | 2005/0227289 A1 | RLIYAASSLQSGVPSRFSGSGSGTEFTLTISSLQPEDFATYYCLQ |
| | | HNTYPPTFGQGTKVEIK (SEQ ID NO: 245) |
| EpoR | VH: SEQ ID | VH |
| | NO: 10 | QVQLQESGPGLVKPSETLSLTCTVSGASISSYYWSWIRQPPGKGL |
| | of U.S. Application | EWIGYIYGSGSTNYNPSLKSRVTISVDTSKNQFSLKLRSVTAADT |
| | NO: US | AVYYCARERLGIGDYWGQGTLVTVSS (SEQ ID NO: 249) |
| | 2005/0227289 A1; | VL: |
| | VL: SEQ ID NO: 17 | DIQLTQSPSSLSASVGDRVTITCRASQGIRNDLGWYQQKPGKAPK |
| | of U.S. Application | RLIYAASSLQSGVPSRFSGSGSGTEFTLTISSLQPEDFATYYCLQ |
| | NO: US | HNTYPPTFGQGTKVEIK (SEQ ID NO: 245) |
| | 2005/0227289 A1 | |
| EpoR | VH: SEQ ID NO: 11 | VH: |
| | of U.S. Application | QVQLQESGPGLVKPSETLSLTCTVSGASISSYYWSWIRQPPGKGL |
| | NO: US | EWIGYIYYEGSTNYNPSLKSRVTISVDTSKNQFSLKLRSVTAADT |
| | 2005/0227289 A1; | AVYYCARERLGIGDYWGQGTLVTVSS (SEQ ID NO: 250) |
| | VL: SEQ ID NO: 17 | VL: |
| | of U.S. Application | DIQLTQSPSSLSASVGDRVTITCRASQGIRNDLGWYQQKPGKAPK |
| | NO: US | RLIYAASSLQSGVPSRFSGSGSGTEFTLTISSLQPEDFATYYCLQ |
| | 2005/0227289 A1 | HNTYPPTFGQGTKVEIK (SEQ ID NO: 245) |
| EpoR | VH: SEQ ID NO: 12 | VH: |
| | of U.S. Application | QVQLQESGPGLVKPSETLSLTCTVSGASISSYYWSWIRQPPGKGL |
| | NO: US | EWIGYIGGSGSTNYNPSLKSRVTISVDTSKNQFSLKLRSVTAADT |
| | 2005/0227289 A1; | AVYYCARERLGIGDYWGQGTLVTVSS (SEQ ID NO: 251) |
| | VL: SEQ ID NO: 17 | VL: |
| | of U.S. Application | DIQLTQSPSSLSASVGDRVTITCRASQGIRNDLGWYQQKPGKAPK |
| | NO: US | RLIYAASSLQSGVPSRFSGSGSGTEFTLTISSLQPEDFATYYCLQ |
| | 2005/0227289 A1 | HNTYPPTFGQGTKVEIK (SEQ ID NO: 245) |
| EpoR | VH: SEQ ID NO: 13 | VH |
| | of U.S. Application | QVQLQESGPGLVKPSETLSLTCTVSGASISSYYWSWIRQPPGKGL |
| | NO: US | EWIGYIYGEGSTNYNPSLKSRVTISVDTSKNQFSLKLRSVTAADT |
| | 2005/0227289 A1; | AVYYCARERLGIGDYWGQGTLVTVSS (SEQ ID NO: 252) |
| | VL: SEQ ID NO: 17 | VL: |
| | of U.S. Application | DIQLTQSPSSLSASVGDRVTITCRASQGIRNDLGWYQQKPGKAPK |
| | NO: US | RLIYAASSLQSGVPSRFSGSGSGTEFTLTISSLQPEDFATYYCLQ |
| | 2005/0227289 A1 | HNTYPPTFGQGTKVEIK (SEQ ID NO: 245) |
| EpoR | VH: SEQ ID NO: 14 | VH: |
| | of U.S. Application | QVQLQESGPGLVKPSETLSLTCTVSGASISSYYWSWIRQPPGKGL |
| | NO: US | EWIGYIGYEGSTNYNPSLKSRVTISVDTSKNQFSLKLRSVTAADT |
| | 2005/0227289 A1; | AVYYCARERLGIGDYWGQGTLVTVSS (SEQ ID NO: 253) |
| | VL: SEQ ID NO: 17 | VL: |
| | of U.S. Application | DIQLTQSPSSLSASVGDRVTITCRASQGIRNDLGWYQQKPGKAPK |
| | NO: US | RLIYAASSLQSGVPSRFSGSGSGTEFTLTISSLQPEDFATYYCLQ |
| | 2005/0227289 A1 | HNTYPPTFGQGTKVEIK (SEQ ID NO: 245) |
| PRLR | VH: SEQ ID NO: 119 | VH: |
| | of PCT Application | DVQLQESGPGLVKPSQSLSLTCTVTGYSITSDYAWNWIRQFPGNK |
| | NO: WO | LEWMGYIGYSGRTSFNPSLKSRISITRDTSKNQFFLQLNSVTTED |
| | 2014/105810 A1; | TATYYCARGGFAMDYWGQGTSVTVSS (SEQ ID NO: 254) |
| | VL: SEQ ID NO: 110 | VL: |
| | of PCT Application | QIVLTQSPGIMSASPGEKVTMTCSASSSVTYMYWYQQKPRSSPKP |
| | NO: WO | WIYLTSNLASGVPARFSGSGSGTSYSLTISSMEAEDGATYYCQQW |
| | 2014/105810 A1 | SSTPPLTFGGGTKLELN (SEQ ID NO: 255) |

### 6.2.4. Targeting Moiety Formats

In certain aspects, a targeting moiety can be any type of antibody or fragment thereof that retains specific binding to an antigenic determinant. Antibody fragments include, but are not limited to, VH (or VH) fragments, VL (or VL) fragments, Fab fragments, F(ab′)2 fragments, scFv fragments, Fv fragments, VHH domains, minibodies, diabodies, triabodies, and tetrabodies.

In some embodiments, the targeting moiety of an engineered IgG molecule share the same format (e.g., Fab, scFv or sdAb). In another embodiment, the targeting moieties do not share the same format.

In some embodiments, the targeting moieties of engineered IgG molecules are Fabs.

In other embodiments, the targeting moieties are scFvs. In yet other embodiments, the targeting moieties are sdAbs.

### 6.2.4.1. Fabs

Fab domains were traditionally produced by proteolytic cleavage of immunoglobulin molecules using enzymes such as papain. In the engineered IgG molecules of the disclosure, the Fab domains can be recombinantly expressed as part of the engineered IgG molecule.

The Fab domains can comprise constant domain and variable region sequences from any suitable species, and thus can be murine, chimeric, human or humanized.

Fab domains typically comprise a CHR domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the engineered IgG molecules of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table F below can be used:

TABLE F
Fab Heterodimerization Strategies

| STRATEGY | VH | CH1 | VL | CL | REFERENCE |
|---|---|---|---|---|---|
| CrossMabC | WT | CL domain | WT | CH1 domain | Schaefer et al., |
| H1-CL | | | | | 2011, Cancer Cell |
| | | | | | 2011; 20: 472-86; |
| | | | | | PMID: 22014573. |
| orthogonal | 39K, 62E | H172A, | 1R, 38D, | L135Y, | Lewis et al., 2014, |
| Fab | | F174G | (36F) | S176W | Nat Biotechnol |
| VHVRD1CH | | | | | 32: 191-8 |
| 1CRD2 - | | | | | |
| VLVRD1Cλ | | | | | |
| CRD2 | | | | | |
| orthogonal | 39Y | WT | 38R | WT | Lewis et al., 2014, |
| Fab | | | | | Nat Biotechnol |
| VHVRD2CH | | | | | 32: 191-8 |
| 1 wt - | | | | | |
| VLVRD2Cλ | | | | | |
| wt | | | | | |
| TCR CαCβ | 39K | TCR Cα | 38D | TCR Cβ | Wu et al., 2015, |
| | | | | | MAbs 7: 364-76 |
| CR3 | WT | T192E | WT | N137K, | Golay at al., 2016, J |
| | | | | S114A | Immunol 196: 3199- |
| | | | | | 211. |
| MUT4 | WT | L143Q, | WT | V133T, | Golay at al., 2016, J |
| | | S188V | | S176V | Immunol 196: 3199- |
| | | | | | 211. |
| DuetMab | WT | F126C | WT | S121C | Mazor et al., 2015, |
| | | | | | MAbs 7: 377-89; |
| | | | | | Mazor et al., 2015, |
| | | | | | MAbs 7: 461-669. |
| Domain | WT | CH3 + knob | WT | CH3 + hole | Wozniak-Knopp et |
| exchanged | | or hole | | or knob | al., 2018, |
| | | mutation | | mutation | PLoSONE13(4): e01 |
| | | | | | 95442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T-cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of engineered IgG molecule of the disclosure. In various embodiments, employing a common light chain as described herein reduces the number of inappropriate species of engineered IgG molecules as compared to employing original cognate VLs. In various embodiments, the VL domains of the engineered IgG molecules are identified from monospecific antibodies comprising a common light chain. In various embodiments, the VH regions of the engineered IgG molecules comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.2.4.2. scFvs

Single chain Fv or “scFv” antibody fragments comprise the VH and VL domains of an antibody in a single polypeptide chain, are capable of being expressed as a single chain polypeptide, and retain the specificity of the intact antibodies from which they are derived.

Generally, the scFv polypeptide further comprises a polypeptide linker between the VH and VL domain that enables the scFv to form the desired structure for target binding.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., typically a repeat of a sequence containing the amino acids glycine and serine, such as the amino acid sequence (Gly4-Ser)₃ (SEQ ID NO:39), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.2.4.3. Single Domain Antibodies

In some embodiments, a targeting moiety of an engineered IgG molecule is a single-domain antibody. A single-domain antibody (sdAb) describes a single antigen-binding domain capable of binding to a cognate antigen. sdAbs are often derived from heavy-chain only antibodies, however they also include single VH domains capable of binding to their cognate antigen in the absence of an associated light chain.

Heavy-chain only antibodies lack both light chains and a functional CH1 domain and thus rely exclusively on a heavy chain variable domain for antigen binding. Heavy-chain only antibodies are produced naturally in the Camelidae family (e.g., camels, dromedaries, llamas, vicunas, guanaco, and alpacas) as well as in cartilaginous fish (e.g., sharks). In addition to natural sources, transgenic mammals (e.g., mice) have been engineered to express heavy-chain only antibodies. Such transgenic mammals include, for example, transgenic animals described in U.S. Patent Publications 2015/0289489 A1, 2023/0270086 A1, and 2023/0062964 A1, and 2020/0267951 A1, each of which is incorporated herein by reference.

In some embodiments, an sdAb is generated by immunizing an animal that produces heavy-chain only antibodies, including a natural producer (e.g., camelids, sharks) or an engineered non-human mammal (e.g., a transgenic mouse), to obtain heavy-chain only antibodies. Such antibodies may be screened to identify those having desirable properties (e.g., target affinity). Once produced and identified, the variable region of the antibody heavy chain is cloned to construct a single domain antibody consisting of only one heavy chain variable region.

sdAbs can also be obtained by immunizing animals that generate traditional antibodies (e.g., rabbits) followed by screening for VHs having high binding affinity in the absence of their cognate light chain (see e.g., Shinozaki et al., 2017, Scientific Reports, 7(1):5794).

sdAbs can be humanized by replacing natural (e.g., camelid) framework sequences with human sequences (see, e.g., Vincke, 2009, The Journal of Biological Chemistry, 285(5):3273-3284; Murakami et al., 2022, Antibodies, 1l(l):10; and U.S. Patent Publication No. 2016/0237142 A1, incorporated herein by reference).

Fully human sdAbs can also be obtained using human VH single domains (see, e.g., Rouet et al., 2015, The Journal of Biological Chemistry, 290(19):11905-11917).

Additional methods for producing heavy-chain only antibodies and/or sdAbs are recognized in the art and include, for example, those described in Muyldermans, 2021, The FEBS journal, 288(7):2084-2102.

In some cases, an sdAb is engineered to enhance certain properties. For example, in some embodiments, a disulfide bond is introduced within a VHH to increase stability (see e.g., Hagihara et al., 2007, The Journal of Biological Chemistry, 282(50):36489-36495).

### 6.2.5. Exemplary Engineered IgG Constructs

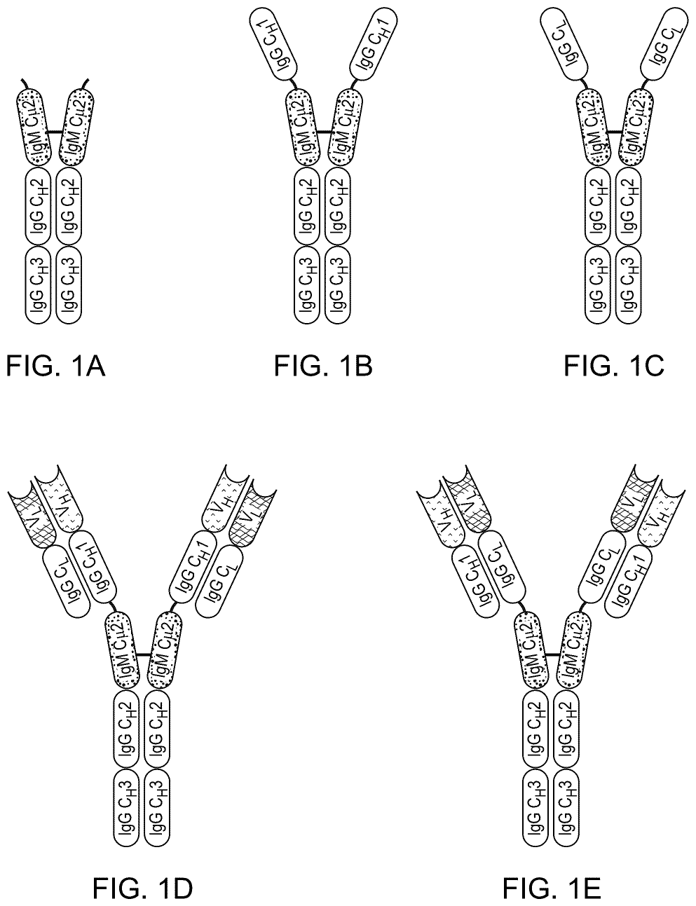

Components of engineered IgG molecules described above can be combined in a variety of ways. Exemplary engineered IgG molecules are illustrated in FIGS. 1A to 1E. The illustrated engineered IgG molecules may include any of the CH1, CH2, CH3, Cμ2, or CL domains or targeting moieties disclosed above, including wild-type or variant versions thereof.

An exemplary Fc region of an engineered IgG molecule is illustrated in FIG. 1A, which depicts a molecule comprising a first polypeptide chain and a second polypeptide chain, each comprising from N-terminus to C-terminus: an IgM Cμ2 domain, an IgG CH2 domain and an IgG CH3 domain. The IgM Cμ2 domains may have the native Cys337 residues which form a disulfide bond between the two heavy chains.

In some embodiments, additional components are present in an engineered IgG molecule that includes the components illustrated in FIG. 1A. For example, one or more of the heavy chains may include: N-terminal to the IgM Cμ2 domain, another IgG constant domain, such as, for example, a CH1 domain or a CL domain, as depicted in FIGS. 1B and 1C, respectively. FIG. 1B depicts a molecule comprising a first polypeptide chain and a second polypeptide chain, each comprising from N-terminus to C-terminus: an IgG CH1 domain, an IgM Cμ2 domain, an IgG CH2 domain and an IgG CH3 domain. FIG. 1C depicts a molecule comprising a first polypeptide chain and a second polypeptide chain, each comprising from N-terminus to C-terminus: an IgG CL domain, an IgM Cμ2 domain, an IgG CH2 domain and an IgG CH3 domain.

In some embodiments, the one or more heavy chains further comprise, N-terminal to the CH1 domain or CL domain, a VH or VL domain, as depicted in FIGS. 1D and 1E, respectively. FIG. 1D depicts a molecule comprising a first polypeptide chain and a second polypeptide chain, each comprising from N-terminus to C-terminus: a VH domain, an IgG CH1 domain, an IgM Cμ2 domain, an IgG CH2 domain and an IgG CH3 domain. The molecule further comprises a third and a fourth polypeptide chain, each comprising from N-terminus to C-terminus: a VL domain and an IgG CL domain. FIG. 1E depicts a molecule comprising a first polypeptide chain and a second polypeptide chain, each comprising from N-terminus to C-terminus: a VL domain, an IgG CL domain, an IgM Cμ2 domain, an IgG CH2 domain and an IgG CH3 domain. The molecule further comprises a third and a fourth polypeptide molecule, each comprising from N-terminus to C-terminus: a VH domain and an IgG CH1 domain.

Example sequences of an IgM Cμ2-IgG Fc portion of certain engineered IgG molecules of the disclosure are provided in Table I, below, with Cμ2 portions underlined. In some embodiments, an engineered IgG molecule comprises IgM Cμ2-IgG Fc portion having at least 90%, at least 95%, at least 98%, at least 99%, or 100% sequence identity with an amino acid sequence set forth in Table I. In some embodiments the engineered IgG molecule further comprises an IgG CH1 domain (e.g., IgG1, IgG2, IgG3, or IgG4 CH1 domain). In some embodiments the engineered IgG molecule further comprises an IgG CL domain (e.g., a K light chain constant domain or a λ light chain constant domain).

TABLE I

| | | SEQ ID |
|---|---|---|
| Structure | Sequence | NO: |
| IgM Cμ2 - | VIAELPPKVSVFVPPRDGFFGNPRKSKLICQATGFSPRQIQVSW | 33 |
| IgG4 Fc | LREGKQVGSGVTTDQVQAEAKESGPTTYKVTSTLTIKESDWLGQ | |
| | SMFTCRVDHRGLTFQQNASSMCVPAPEFLGGPSVFLFPPKPKDT | |
| | LMISRTPEVTCVVVDVSQEDPEVQFNWYVDGVEVHNAKTKPREE | |
| | QFNSTYRVVSVLTVLHQDWLNGKEYKCKVSNKGLPSSIEKTISK | |
| | AKGQPREPQVYTLPPSQEEMTKNQVSLTCLVKGFYPSDIAVEWE | |
| | SNGQPENNYKTTPPVLDSDGSFFLYSRLTVDKSRWQEGNVFSCS | |
| | VMHEALHNHYTQKSLSLSLGK | |
| IgM Cμ2 - | VIAELPPKVSVFVPPRDGFFGNPRKSKLICQATGFSPRQIQVSW | 34 |
| IgG2 Fc | LREGKQVGSGVTTDQVQAEAKESGPTTYKVTSTLTIKESDWLGQ | |
| | SMFTCRVDHRGLTFQQNASSMCVPAPPVAGPSVFLFPPKPKDTL | |
| | MISRTPEVTCVVVDVSHEDPEVQFNWYVDGVEVHNAKTKPREEQ | |
| | FNSTFRVVSVLTVVHQDWLNGKEYKCKVSNKGLPAPIEKTISKT | |
| | KGQPREPQVYTLPPSREEMTKNQVSLTCLVKGFYPSDIAVEWES | |
| | NGQPENNYKTTPPMLDSDGSFFLYSKLTVDKSRWQQGNVFSCSV | |
| | MHEALHNHYTQKSLSLSPGK | |
| IgM (+PLP) | PLPVIAELPPKVSVFVPPRDGFFGNPRKSKLICQATGFSPRQIQ | 35 |
| Cμ2 - IgG4 | VSWLREGKQVGSGVTTDQVQAEAKESGPTTYKVTSTLTIKESDW | |
| Fc | LGQSMFTCRVDHRGLTFQQNASSMCVPAPEFLGGPSVFLFPPKP | |
| | KDTLMISRTPEVTCVVVDVSQEDPEVQFNWYVDGVEVHNAKTKP | |
| | REEQFNSTYRVVSVLTVLHQDWLNGKEYKCKVSNKGLPSSIEKT | |
| | ISKAKGQPREPQVYTLPPSQEEMTKNQVSLTCLVKGFYPSDIAV | |
| | EWESNGQPENNYKTTPPVLDSDGSFFLYSRLTVDKSRWQEGNVF | |
| | SCSVMHEALHNHYTQKSLSLSLGK | |
| IgM (+PLP) | PLPVIAELPPKVSVFVPPRDGFFGNPRKSKLICQATGFSPRQIQ | 36 |
| Cμ2 - IgG2 | VSWLREGKQVGSGVTTDQVQAEAKESGPTTYKVTSTLTIKESDW | |
| Fc | LGQSMFTCRVDHRGLTFQQNASSMCVPAPPVAGPSVFLFPPKPK | |
| | DTLMISRTPEVTCVVVDVSHEDPEVQFNWYVDGVEVHNAKTKPR | |
| | EEQFNSTFRVVSVLTVVHQDWLNGKEYKCKVSNKGLPAPIEKTI | |
| | SKTKGQPREPQVYTLPPSREEMTKNQVSLTCLVKGFYPSDIAVE | |
| | WESNGQPENNYKTTPPMLDSDGSFFLYSKLTVDKSRWQQGNVFS | |
| | CSVMHEALHNHYTQKSLSLSPGK | |
| IgM (+PLP) | PLPVIAELPPKVSVFVPPRDGFFGNPRKSKLICQATGFSPRQIQ | 37 |
| Cμ2 - (−AP) | VSWLREGKQVGSGVTTDQVQAEAKESGPTTYKVTSTLTIKESDW | |
| IgG4 Fc | LGQSMFTCRVDHRGLTFQQNASSMCVPEFLGGPSVFLFPPKPKD | |
| | TLMISRTPEVTCVVVDVSQEDPEVQFNWYVDGVEVHNAKTKPRE | |
| | EQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEW | |
| | ESNGQPENNYKTTPPVLDSDGSFFLYSRLTVDKSRWQEGNVFSC | |
| | SVMHEALHNHYTQKSLSLSLGK | |
| IgM (+PLP) | PLPVIAELPPKVSVFVPPRDGFFGNPRKSKLICQATGFSPRQIQ | 38 |
| Cμ2 - (−AP) | VSWLREGKQVGSGVTTDQVQAEAKESGPTTYKVTSTLTIKESDW | |
| IgG4 Fc | LGQSMFTCRVDHRGLTFQQNASSMCVPPVAGPSVFLFPPKPKDT | |
| | LMISRTPEVTCVVVDVSHEDPEVQFNWYVDGVEVHNAKTKPREE | |
| | QFNSTFRVVSVLTVVHQDWLNGKEYKCKVSNKGLPAPIEKTISK | |
| | TKGQPREPQVYTLPPSREEMTKNQVSLTCLVKGFYPSDIAVEWE | |
| | SNGQPENNYKTTPPMLDSDGSFFLYSKLTVDKSRWQQGNVFSCS | |
| | VMHEALHNHYTQKSLSLSPGK | |

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 33. In some embodiments, the engineered IgG molecule comprises the amino acid sequence of SEQ ID NO: 33. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 33. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 33.

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 34. In some embodiments, the engineered IgG molecule comprises the amino acid sequence of SEQ ID NO: 34. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 34. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 34.

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 35. In some embodiments, the engineered IgG molecule comprises the amino acid sequence of SEQ ID NO: 35. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 35. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 35.

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 36. In some embodiments, the engineered IgG molecule comprises the amino acid sequence of SEQ ID NO: 36. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 36. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 36.

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 37. In some embodiments, the engineered IgG molecule comprises the amino acid sequence of SEQ ID NO: 37. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 37. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 37.

In some embodiments, engineered IgG molecules comprise an IgM Cμ2-IgG Fc portion having at least 90%, at least 95%, at least 98%, at least 99%, or 100% sequence identity to the amino acid sequence of SEQ ID NO: 38. In some embodiments, the engineered IgG molecule comprises the amino acid sequence of SEQ ID NO: 38. In some embodiments the engineered IgG molecule further comprises an IgG CH1 domain (e.g., IgG1, IgG2, IgG3, or IgG4 CH1 domain)N-terminal (e.g., immediately N-terminal) to the amino acid sequence having at least 90%, at least 95%, at least 98%, at least 99%, or 100% sequence identity to the amino acid sequence of SEQ ID NO: 38. In some embodiments the engineered IgG molecule further comprises an IgG CL domain (e.g., a K light chain constant domain or a λ light chain constant domain)N-terminal (e.g., immediately N-terminal) to the amino acid sequence having at least 90%, at least 95%, at least 98%, at least 99%, or 100% sequence identity to the amino acid sequence of SEQ ID NO: 38.

### 6.3. Nucleic Acids and Host Cells

In another aspect, the disclosure provides nucleic acids encoding the engineered IgG molecules of the disclosure and/or their individual components. In some embodiments, the components of engineered IgG molecules (e.g., heavy and light chains) are encoded by a single nucleic acid. In other embodiments, components of the engineered IgG molecules are encoded by separate nucleic acids.

For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.3.1. Vectors

The disclosure provides vectors comprising nucleotide sequences encoding engineered IgG molecules or components thereof described herein, for example one or two of the polypeptide chains of an engineered IgG molecule. The vectors may include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.3.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.4. Pharmaceutical Compositions

The engineered IgG molecules of the disclosure may be in the form of compositions comprising the engineered IgG molecules and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the engineered IgG molecules and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject.

Typically, the pharmaceutical composition will be administered intravenously or subcutaneously.

Pharmaceutical compositions can be conveniently presented in unit dosage forms containing a predetermined amount of engineered IgG molecule per dose. The quantity of the engineered IgG molecule in a unit dose will depend on the disease being treated, as well as other factors as are well known in the art. Such unit dosages may be in the form of a lyophilized dry powder containing an amount of the engineered IgG molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of the engineered IgG molecule suitable for a single administration.

The pharmaceutical compositions may also be supplied in bulk from containing quantities of the engineered IgG molecule suitable for multiple administrations.

When formulated into a single formulation, the engineered IgG molecule can be used in approximately equimolar quantities.

Pharmaceutical compositions may be prepared for storage as lyophilized formulations or aqueous solutions by mixing an engineered IgG molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be non-toxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10% w/w of engineered IgG.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.5. Therapeutic Indications and Methods of Treatment

The present disclosure provides methods for using and applications for the engineered IgG molecules of the disclosure.

In some embodiments, engineered IgG molecules of the present disclosure may be receptor agonists with enhanced agonistic activity relative to parental IgG molecules that lack an IgM Cμ2 domain and/or that have a native IgG hinge region. Engineered IgG molecules of the disclosure may be useful in treating disease states where stimulation of a cell membrane receptor may be beneficial, including, for example, conditions where an enhanced receptor response is desirable. These may include disease states where the host has a defect related to signaling through a receptor, such as a deficiency in the receptor or receptor ligand. Deficiencies in the receptor ligand may include a lower-than-normal concentration of the receptor ligand or a mutation in the ligand that makes it less effective at stimulating signaling through the receptor. Other therapeutic uses for the engineered IgG molecules of the present disclosure may include enhancing immune activation by agonizing stimulatory immune receptors and/or cytokine receptors.

In one aspect, engineered IgG molecules of the disclosure for use as a medicament are provided. In further aspects, engineered IgG molecules of the disclosure for use in treating a disease or condition are provided. In certain embodiments, engineered IgG molecules of the disclosure for use in a method of treatment are provided. In one embodiment, the disclosure provides engineered IgG molecules as described herein for use in the treatment of a disease or condition in a subject in need thereof. In certain embodiments, the disclosure provides engineered IgG molecules for use in a method of treating a subject having a disease or condition comprising administering to the individual a therapeutically effective amount of the engineered IgG molecule. In certain embodiments the disease or condition to be treated is a metabolic disorder. In a preferred embodiment the disease or condition is obesity. In some embodiments, the disease or condition is associated with or caused by leptin deficiency or leptin resistance. In some embodiments, the disease or condition is metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome, or Rabson-Mendenhall syndrome. In some embodiments, In some embodiments, the disease or condition is generalized lipodystrophy, acquired generalized lipodystrophy, familial partial lipodystrophy, acquired partial lipodystrophy, centrifugal abdominal lipodystrophy, lipoatrophia annularis, localized lipodystrophy, or HIV-associated lipodystrophy. In certain embodiments the method further comprises administering to the individual a therapeutically effective amount of at least one additional therapeutic agent. In further embodiments, the disclosure provides an engineered IgG molecule for use in stimulating the immune system. In certain embodiments, the disclosure provides an engineered IgG molecule for use in a method of stimulating the immune system in a subject comprising administering to the individual an effective amount of an engineered IgG molecule to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors, an increase in T-cell responsiveness, an increase in natural killer cell activity or lymphokine-activated killer (LAK) cell activity, and the like.

In a further aspect, the disclosure provides for the use of an engineered IgG molecule of the disclosure in the manufacture or preparation of a medicament for the treatment of a disease or condition in a subject in need thereof. In one embodiment, the medicament is for use in a method of treating a disease or condition comprising administering to a subject having the disease or condition a therapeutically effective amount of the medicament. In certain embodiments the disease to be treated is a metabolic disorder.

In a preferred embodiment the disease is obesity. In one such embodiment, the method further comprises administering to the individual a therapeutically effective amount of at least one additional therapeutic agent. In a further embodiment, the medicament is for stimulating the immune system. In a further embodiment, the medicament is for use in a method of stimulating the immune system in a subject comprising administering to the individual an amount effective of the medicament to stimulate the immune system. In certain embodiments the disease to be treated is a proliferative disorder such as, for example, cancer. In one such embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors or other immunostimulatory receptors, an increase in T-cell responsiveness, an increase in natural killer cell activity or lymphokine-activated killer (LAK) cell activity, and the like.

Further provided are methods of agonizing a target molecule such as, for example, a membrane receptor, by contacting the target molecule with an engineered IgG molecule of the disclosure. The target molecule may be present in or on the surface of a cultured cell or in or on the surface of a cell within an individual. The target molecule to be agonized in such methods may include any of the target molecules disclosed herein.

An “individual” according to any of the above embodiments may be a mammal, preferably a human. “Stimulation of the immune system” according to any of the above embodiments may include any one or more of a general increase in immune function, an increase in T-cell function, an increase in B cell function, a restoration of lymphocyte function, an increase in the expression of IL2 receptors or other immunostimulatory receptors, an increase in T-cell responsiveness, an increase in natural killer cell activity or lymphokine-activated killer (LAK) cell activity, and the like.

Engineered IgG molecules of the disclosure, in particular those that target TNF family receptors such as, for example, CD40, OX40, GITR, 4-1BB, CD27, HVEM, or CD30, may be useful in treating disease states where stimulation of the immune system of the host is beneficial, in particular conditions where an enhanced cellular immune response is desirable. These may include disease states where the host immune response is insufficient or deficient.

Disease states for which the engineered IgG molecules of the disclosure can be administered comprise, for example, a tumor or infection where a cellular immune response would be a critical mechanism for specific immunity. Specific disease states for which engineered IgG molecules of the present disclosure can be employed include cancer, including breast cancer, prostate cancer, and colorectal cancer. The engineered IgG molecules of the disclosure may be administered per se or in any suitable pharmaceutical composition.

In various embodiments, the engineered IgG molecules of the disclosure are useful for the treatment of cancer, for the prevention or treatment of metastasis, for stimulating the formation, stability and/or activity of a cytotoxic immune synapse, for inducing tumor cytolysis, for inducing anti-tumor cytotoxicity, for stimulating an immune response against a tumor, or any combination of two or more of the foregoing uses.

In a further aspect, the disclosure provides a method for treating a disease in a subject, comprising administering to said individual a therapeutically effective amount of an engineered IgG molecule of the disclosure. In one embodiment, a composition comprising an engineered IgG molecule in a pharmaceutically acceptable form is administered to said individual. In certain embodiments the disease to be treated by administering engineered IgG molecules of the present disclosure is a proliferative disorder. In some embodiments, the disease is cancer. In certain embodiments the method further comprises administering to the individual a therapeutically effective amount of at least one additional therapeutic agent, e.g., an anti-cancer agent if the disease to be treated is cancer. In further embodiments, the disclosure provides a tumor-targeted engineered IgG molecule for use in stimulating the immune system. In certain embodiments, the disclosure provides a tumor-targeted engineered IgG molecule for use in a method of stimulating the immune system in a subject comprising administering to the individual an effective amount of the tumor-targeted engineered IgG molecule to stimulate the immune system.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using an engineered IgG molecule of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments, the cancer is chosen from the group consisting of renal cell cancer, skin cancer, lung cancer, colorectal cancer, breast cancer, brain cancer, head, and neck cancer.

Similarly, other cell proliferation disorders can also be treated by the engineered IgG molecules of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

The present disclosure further provides a method of localized delivery of an engineered IgG molecule, comprising administering to a subject an engineered IgG molecule as described herein, where engineered IgG molecule comprises a targeting moiety that recognizes a target molecule that is expressed by a tissue to which the engineered IgG molecule is to be locally delivered. As used herein, the term “locally delivered” does not require local administration but rather indicates that the engineered IgG molecule be selectively localized to a tissue of interest following administration. In some embodiments, the administration is not local to the tissue.

A skilled artisan readily recognizes that in many cases the engineered IgG molecule may not provide a cure but may only provide partial benefit. In some embodiments, a physiological change having some benefit is also considered therapeutically beneficial.

Thus, in some embodiments, an amount of an engineered IgG molecule that provides a physiological change is considered an “effective amount” or a “therapeutically effective amount.”

The subject, patient, or individual in need of treatment is typically a mammal, more specifically a human.

The appropriate dosage of an engineered IgG molecule of the disclosure (when used alone or in combination with one or more other additional therapeutic agents) will depend on the type of disease to be treated, the route of administration, the body weight of the patient, the particular engineered IgG molecule, the severity and course of the disease, whether the antibody is administered for preventive or therapeutic purposes, previous or concurrent therapeutic interventions, the patient's clinical history and response to the engineered IgG molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A therapeutically effective amount of an engineered IgG molecule may comprise only a single administration or many administrations over a period of time. Thus, an engineered IgG molecule is suitably administered to the patient at one time or over a series of treatments. Depending on the type and severity of the disease, about 1 μg/kg to 15 mg/kg (e.g., 0.1 mg/kg-10 mg/kg) of an engineered IgG molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an engineered IgG molecule would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the engineered IgG molecule). An initial higher loading dose, followed by one or more lower doses, may be administered. However, other dosage regimens may be useful. The progress of this therapy is easily monitored by conventional techniques and assays.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the engineered IgG molecule which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the engineered IgG molecule may not be related to plasma concentration. One having skill in the art will be able to optimize therapeutically effective local dosages without undue experimentation.

### 6.6. Combination Therapy

The engineered IgG molecules disclosed herein may be administered in combination with one or more other agents in therapy. For instance, an engineered IgG molecule of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of engineered IgG molecule used, the type of disorder or treatment, and other factors discussed above. The engineered IgG molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the engineered IgG molecule can occur prior to, simultaneously, and/or following, administration of the additional therapeutic agent and/or adjuvant.

### 7. SPECIFIC EMBODIMENTS

The present disclosure is exemplified by the specific embodiments below.

1. A molecule comprising a first polypeptide chain comprising, in N- to C-terminal orientation:
(a) an IgG constant domain;
(b) an IgM Cμ2 domain;
(c) an IgG CH2 domain; and
(d) an IgG CH3 domain.

2. The molecule of embodiment 1, wherein the first polypeptide chain lacks an intact IgG hinge domain.

3. The molecule of embodiment 1 or 2, wherein the first polypeptide chain has an IgG hinge domain of reduced length relative to a wild type IgG hinge domain.

4. The molecule of embodiment 2 or 3, wherein the IgG hinge domain is an IgG1 hinge domain.

5. The molecule of embodiment 4, wherein the first polypeptide chain lacks an amino acid sequence having at least 90% sequence identity to the amino acid sequence of SEQ ID NO:3.

6. The molecule of embodiment 2 or 3, wherein the IgG hinge domain is an IgG2 hinge domain.

7. The molecule of embodiment 5, wherein the first polypeptide chain lacks an amino acid sequence having at least 90% sequence identity to the amino acid sequence of SEQ ID NO:4.

8. The molecule of embodiment 2 or 3, wherein the IgG hinge domain is an IgG4 hinge domain.

9. The molecule of embodiment 8, wherein the first polypeptide chain lacks an amino acid sequence having at least 90% sequence identity to the amino acid sequence of SEQ ID NO:5.

10. The molecule of any one of embodiments 1 to 9, which lacks an IgM Cμ1 domain.

11. The molecule of embodiment 10, wherein the first polypeptide chain lacks an amino acid sequence having at least 90% sequence identity to the amino acid sequence of SEQ ID NO:2.

12. The molecule of any one of embodiments 1 to 11, wherein the IgG constant domain is an IgG CH1 domain.

13. The molecule of embodiment 12, wherein the IgG CH1 domain is an IgG1 CH1 domain.

14. The molecule of embodiment 12, wherein the IgG CH1 domain is an IgG2 CH1 domain.

15. The molecule of embodiment 12, wherein the IgG CH1 domain is an IgG4 CH1 domain.

16. The molecule of any one of embodiments 12 to 15, wherein the first polypeptide chain further comprises, N-terminal to the IgG CH1 domain, a VH domain.

17. The molecule of any one of embodiments 1 to 11, wherein the IgG constant domain is an IgG CL domain.

18. The molecule of embodiment 17, wherein the first polypeptide chain further comprises, N-terminal to the IgG CL domain, a VL domain.

19. The molecule of any one of embodiments 1 to 18, wherein the IgG CH2 domain is an IgG1 CH2 domain.

20. The molecule of any one of embodiments 1 to 18, wherein the IgG CH2 domain is an IgG2 CH2 domain.

21. The molecule of any one of embodiments 1 to 18, wherein the IgG CH2 domain is an IgG4 CH2 domain.

22. The molecule of any one of embodiments 1 to 21, wherein the IgG CH3 domain is an IgG1 CH3 domain.

23. The molecule of any one of embodiments 1 to 21, wherein the IgG CH3 domain is an IgG2 CH3 domain.

24. The molecule of any one of embodiments 1 to 21, wherein the IgG CH3 domain is an IgG4 CH3 domain.

25. The molecule of any one of embodiments 1 to 24, wherein the IgG CH2 domain comprises one or more amino acid substitutions that reduce binding to an Fc receptor.

26. The molecule of embodiment 25, wherein the Fc receptor is an Fcγ receptor.

27. The molecule of embodiment 26, wherein the Fc receptor is FcγRIIIa.

28. The molecule of any one of embodiments 1 to 24, wherein the IgG CH2 domain comprises one or more amino acid substitutions that reduce effector function.

29. The molecule of any one of embodiments 1 to 24, wherein the IgG CH2 domain comprises one or more amino acid substitutions that increase binding to an Fc receptor.

30. The molecule of embodiment 29, wherein the Fc receptor is FcγRIIB.

31. The molecule of any one of embodiments 1 to 30, wherein the IgG CH3 domain comprises one or more amino acid substitutions that reduce binding to an Fc receptor.

32. The molecule of embodiment 31, wherein the Fc receptor is an Fcγ receptor.

33. The molecule of embodiment 32, wherein the Fc receptor is FcγRIIIa.

34. The molecule of any one of embodiments 1 to 30, wherein the IgG CH3 domain comprises one or more amino acid substitutions that reduce effector function.

35. The molecule of any one of embodiments 1 to 30, wherein the IgG CH3 domain comprises one or more amino acid substitutions that increase binding to an Fc receptor.

36. The molecule of embodiment 35, wherein the Fc receptor is FcγRIIB.

37. The molecule of any one of embodiments 1 to 36, wherein the IgG CH2 domain and IgG CH3 domain together comprise an amino acid sequence having at least 90% sequence identity to an amino acid sequence set forth in Table F-1.

38. The molecule of any one of embodiments 1 to 36, wherein the IgG CH2 domain and IgG CH3 domain together comprise an amino acid sequence having at least 95% sequence identity to an amino acid sequence set forth in Table F-1.

39. The molecule of any one of embodiments 1 to 36, wherein the IgG CH2 domain and IgG CH3 domain together comprise an amino acid sequence having at least 98% sequence identity to an amino acid sequence set forth in Table F-1.

40. The molecule of any one of embodiments 1 to 36, wherein the IgG CH2 domain and IgG CH3 domain together comprise an amino acid sequence having at least 99% sequence identity to an amino acid sequence set forth in Table F-1.

41. The molecule of any one of embodiments 1 to 36, wherein the IgG CH2 domain and IgG CH3 domain together comprise an amino acid sequence set forth in Table F-1.

42. The molecule of any one of embodiments 1 to 41, wherein the first polypeptide chain comprises an amino acid sequence having at least 90% sequence identity to the amino acid sequence of SEQ ID NO:34.

43. The molecule of any one of embodiments 1 to 41, wherein the first polypeptide chain comprises an amino acid sequence having at least 95% sequence identity to the amino acid sequence of SEQ ID NO:34.

44. The molecule of any one of embodiments 1 to 41, wherein the first polypeptide chain comprises an amino acid sequence having at least 98% sequence identity to the amino acid sequence of SEQ ID NO:34.

45. The molecule of any one of embodiments 1 to 41, wherein the first polypeptide chain comprises an amino acid sequence having at least 99% sequence identity to the amino acid sequence of SEQ ID NO:34.

46. The molecule of any one of embodiments 1 to 41, wherein the first polypeptide chain comprises an amino acid sequence having at least 99.5% sequence identity to the amino acid sequence of SEQ ID NO:34.

47. The molecule of any one of embodiments 1 to 41, wherein the first polypeptide chain comprises the amino acid sequence of SEQ ID NO:34.

48. The molecule of any one of embodiments 1 to 41, wherein the first polypeptide chain comprises an amino acid sequence having at least 90% sequence identity to the amino acid sequence of SEQ ID NO:33.

49. The molecule of any one of embodiments 1 to 41, wherein the first polypeptide chain comprises an amino acid sequence having at least 95% sequence identity to the amino acid sequence of SEQ ID NO:33.

50. The molecule of any one of embodiments 1 to 41, wherein the first polypeptide chain comprises an amino acid sequence having at least 98% sequence identity to the amino acid sequence of SEQ ID NO:33.

51. The molecule of any one of embodiments 1 to 41, wherein the first polypeptide chain comprises an amino acid sequence having at least 99% sequence identity to the amino acid sequence of SEQ ID NO:33.

52. The molecule of any one of embodiments 1 to 41, wherein the first polypeptide chain comprises an amino acid sequence having at least 99.5% sequence identity to the amino acid sequence of SEQ ID NO:33.

53. The molecule of any one of embodiments 1 to 41, wherein the first polypeptide chain comprises the amino acid sequence of SEQ ID NO:33.

54. The molecule of any one of embodiments 1 to 18, which comprises an Fc dimer.

55. The molecule of embodiment 54, wherein the Fc dimer is an Fc homodimer.

56. The molecule of any one of embodiments 1 to 55, further comprising a second polypeptide chain comprising, in N- to C-terminal orientation:
(a) an IgG constant domain;
(b) an IgM Cμ2 domain;
(c) an IgG CH2 domain; and
(d) an IgG CH3 domain.

57. The molecule of embodiment 56, wherein the second polypeptide chain lacks an intact IgG hinge domain.

58. The molecule of embodiment 56 or 57, wherein the second polypeptide chain has an IgG hinge domain of reduced length relative to a wild type IgG hinge domain.

59. The molecule of embodiment 57 or 58, wherein the IgG hinge domain is an IgG1 hinge domain.

60. The molecule of embodiment 59, wherein the second polypeptide chain lacks an amino acid sequence having at least 90% sequence identity to the amino acid sequence of SEQ ID NO:3.

61. The molecule of embodiment 57 or 58, wherein the IgG hinge domain is an IgG2 hinge domain.

62. The molecule of embodiment 61, wherein the second polypeptide chain lacks an amino acid sequence having at least 90% sequence identity to the amino acid sequence of SEQ ID NO:4.

63. The molecule of embodiment 57 or 58, wherein the IgG hinge domain is an IgG4 hinge domain.

64. The molecule of embodiment 63, wherein the second polypeptide chain lacks an amino acid sequence having at least 90% sequence identity to the amino acid sequence of SEQ ID NO:5.

65. The molecule of any one of embodiments 56 to 64, which lacks an IgM Cμ1 domain.

66. The molecule of embodiment 65, wherein the second polypeptide chain lacks an amino acid sequence having at least 90% sequence identity to the amino acid sequence of SEQ ID NO:2.

67. The molecule of any one of embodiments 56 to 66, wherein the IgG constant domain is an IgG CH1 domain.

68. The molecule of embodiment 67, wherein the IgG CH1 domain is an IgG2 CH1 domain.

69. The molecule of embodiment 67, wherein the IgG CH1 domain is an IgG4 CH1 domain.

70. The molecule of embodiment 67, wherein the IgG CH2 domain is an IgG2 CH2 domain.

71. The molecule of embodiment 67, wherein the IgG CH2 domain is an IgG4 CH2 domain.

72. The molecule of any one of embodiments 67 to 71, wherein the first polypeptide chain further comprises, N-terminal to the IgG CH1 domain, a VH domain.

73. The molecule of any one of embodiments 56 to 66, wherein the IgG constant domain is an IgG CL domain.

74. The molecule of embodiment 73, wherein the first polypeptide chain further comprises, N-terminal to the IgG CL domain, a VL domain.

75. The molecule of any one of embodiments 56 to 74, wherein the IgG CH3 domain is an IgG2 CH3 domain.

76. The molecule of any one of embodiments 56 to 74, wherein the IgG CH3 domain is an IgG4 CH3 domain.

77. The molecule of any one of embodiments 56 to 74, wherein the IgG CH2 domain comprises one or more amino acid substitutions that reduce binding to an Fc receptor.

78. The molecule of embodiment 77, wherein the Fc receptor is an Fcγ receptor.

79. The molecule of embodiment 78, wherein the Fc receptor is FcγRIIIa.

80. The molecule of any one of embodiments 56 to 74, wherein the IgG CH2 domain comprises one or more amino acid substitutions that reduce effector function.

81. The molecule of any one of embodiments 56 to 74, wherein the IgG CH2 domain comprises one or more amino acid substitutions that increase binding to an Fc receptor.

82. The molecule of embodiment 81, wherein the Fc receptor is FcγRIIB.

83. The molecule of any one of embodiments 56 to 82, wherein the IgG CH3 domain comprises one or more amino acid substitutions that reduce binding to an Fc receptor.

84. The molecule of embodiment 83, wherein the Fc receptor is an Fcγ receptor.

85. The molecule of embodiment 84, wherein the Fc receptor is FcγRIIIa.

86. The molecule of any one of embodiments 56 to 82, wherein the IgG CH3 domain comprises one or more amino acid substitutions that reduce effector function.

87. The molecule of any one of embodiments 56 to 82, wherein the IgG CH3 domain comprises one or more amino acid substitutions that increase binding to an Fc receptor.

88. The molecule of embodiment 87, wherein the Fc receptor is FcγRIIB.

89. The molecule of any one of embodiments 56 to 88, wherein the IgG CH2 domain and IgG CH3 domain together comprise an amino acid sequence having at least 90% sequence identity to an amino acid sequence set forth in Table F-1.

90. The molecule of any one of embodiments 56 to 88, wherein the IgG CH2 domain and IgG CH3 domain together comprise an amino acid sequence having at least 95% sequence identity to an amino acid sequence set forth in Table F-1.

91. The molecule of any one of embodiments 56 to 88, wherein the IgG CH2 domain and IgG CH3 domain together comprise an amino acid sequence having at least 98% sequence identity to an amino acid sequence set forth in Table F-1.

92. The molecule of any one of embodiments 56 to 88, wherein the IgG CH2 domain and IgG CH3 domain together comprise an amino acid sequence having at least 99% sequence identity to an amino acid sequence set forth in Table F-1.

93. The molecule of any one of embodiments 56 to 88, wherein the IgG CH2 domain and IgG CH3 domain together comprise an amino acid sequence set forth in Table F-1.

94. The molecule of any one of embodiments 56 to 93, which comprises an amino acid sequence having at least 90% sequence identity to the amino acid sequence of SEQ ID NO:34.

95. The molecule of any one of embodiments 56 to 93, which comprises an amino acid sequence having at least 95% sequence identity to the amino acid sequence of SEQ ID NO:34.

96. The molecule of any one of embodiments 56 to 93, which comprises an amino acid sequence having at least 98% sequence identity to the amino acid sequence of SEQ ID NO:34.

97. The molecule of any one of embodiments 56 to 93, which comprises an amino acid sequence having at least 99% sequence identity to the amino acid sequence of SEQ ID NO:34.

98. The molecule of any one of embodiments 56 to 93, which comprises an amino acid sequence having at least 99.5% sequence identity to the amino acid sequence of SEQ ID NO:34.

99. The molecule of any one of embodiments 56 to 93, which comprises the amino acid sequence of SEQ ID NO:34.

100. The molecule of any one of embodiments 56 to 93, which comprises an amino acid sequence having at least 90% sequence identity to the amino acid sequence of SEQ ID NO:33.

101. The molecule of any one of embodiments 56 to 93, which comprises an amino acid sequence having at least 95% sequence identity to the amino acid sequence of SEQ ID NO:33.

102. The molecule of any one of embodiments 56 to 93, which comprises an amino acid sequence having at least 98% sequence identity to the amino acid sequence of SEQ ID NO:33.

103. The molecule of any one of embodiments 56 to 93, which comprises an amino acid sequence having at least 99% sequence identity to the amino acid sequence of SEQ ID NO:33.

104. The molecule of any one of embodiments 56 to 93, which comprises an amino acid sequence having at least 99.5% sequence identity to the amino acid sequence of SEQ ID NO:33.

105. The molecule of any one of embodiments 56 to 93, which comprises the amino acid sequence of SEQ ID NO:33.

106. The molecule of any one of embodiments 1 to 105, wherein the molecule is a binding molecule.

107. The molecule of embodiment 106, which comprises a first targeting moiety.

108. The molecule of embodiment 107, wherein the first targeting moiety is a Fab.

109. The molecule of any one of embodiments 106 to 108, which comprises:
(a) a first polypeptide chain comprising, from N- to C-terminal orientation:
   (i) a first targeting moiety or portion thereof;
   (ii) a first IgM Cμ2 domain;
   (iii) a first IgG CH2 domain; and
   (iv) a first IgG CH3 domain;
(b) a second polypeptide chain comprising, from N- to C-terminal orientation:
   (i) a second targeting moiety or portion thereof;
   (ii) a second IgM Cμ2 domain;
   (iii) a second IgG CH2 domain; and
   (iv) a second IgG CH3 domain.

110. The molecule of embodiment 109, wherein the first polypeptide chain comprises, N-terminal to the first IgM Cμ2 domain, a first VH domain and a first IgG CH1 domain.

111. The molecule of embodiment 110, further comprising a third polypeptide chain comprising a first VL domain and a first IgG CL domain, the third polypeptide chain associated with the first polypeptide chain to form the first targeting moiety.

112. The molecule of embodiment 109, wherein the first polypeptide chain comprises, N-terminal to the first IgM Cμ2 domain, a first VL domain and a first IgG CL domain.

113. The molecule of embodiment 112, further comprising a third polypeptide chain comprising a first VH domain and a first IgG CH1 domain, the third polypeptide chain associated with the first polypeptide chain to form the first targeting moiety.

114. The molecule of any one of embodiments 109 to 113, wherein the second polypeptide chain comprises, N-terminal to the first IgM Cμ2 domain, a first VH domain and a first IgG CH1 domain.

115. The molecule of embodiment 114, further comprising a fourth polypeptide chain comprising a second VL domain and a second IgG CL domain, the fourth polypeptide chain associated with the second polypeptide chain to form the second targeting moiety.

116. The molecule of any one of embodiments 109 to 113, wherein the second polypeptide chain comprises, N-terminal to the first IgM Cμ2 domain, a first VL domain and a first IgG CL domain.

117. The molecule of embodiment 116, further comprising a fourth polypeptide chain comprising a second VH domain and a second IgG CH1 domain, the fourth polypeptide chain associated with the second polypeptide chain to form the second targeting moiety.

118. The molecule of any one of embodiments 107 to 117, wherein the first targeting moiety and/or second targeting moiety comprises an antigen binding domain of an agonist antibody.

119. The molecule of any one of embodiments 107 to 110, wherein the first targeting moiety and/or second targeting moiety comprises an antigen binding domain of an antibody set forth in Table B.

120. The molecule of any one of embodiments 107 to 119, wherein the first targeting moiety and/or second targeting moiety binds to a target molecule.

121. The molecule of embodiment 120, wherein the target molecule is a receptor.

122. The molecule of embodiment 121, wherein the target molecule is CD40, OX40, GITR, 4-1BB, CD27, HVEM, CD30, leptin receptor, or IGF1R.

123. The molecule of embodiment 121, wherein the target molecule is a TNF family receptor.

124. The molecule of embodiment 123, wherein the target molecule is CD40, OX40, GITR, 4-1BB, CD27, HVEM, or CD30.

125. The molecule of embodiment 121, wherein the target molecule is a homodimer class I cytokine receptor.

126. The molecule of embodiment 125, wherein the target molecule is EPO receptor, G-CSF receptor, leptin receptor (LEPR), or PRLR.

127. The molecule of embodiment 125 or 126, wherein the target molecule is leptin receptor (LEPR).

128. A molecule, optionally the molecule of any one of embodiments 1 to 127, comprising a polypeptide chain comprising an amino acid sequence having at least 98% sequence identity to the amino acid sequence of SEQ ID NO:33.

129. The molecule of claim 128, wherein the molecule comprises a polypeptide chain comprising an amino acid sequence having at least 99% sequence identity to the amino acid sequence of SEQ ID NO:33.

130. The molecule of claim 128, wherein the molecule comprises a polypeptide chain comprising the amino acid sequence of SEQ ID NO:33.

131. A molecule, optionally the molecule of any one of embodiments 1 to 127, comprising a polypeptide chain comprising an amino acid sequence having at least 98% sequence identity to the amino acid sequence of SEQ ID NO:34.

132. The molecule of claim 131, wherein the molecule comprises a polypeptide chain comprising an amino acid sequence having at least 99% sequence identity to the amino acid sequence of SEQ ID NO:34.

133. The molecule of claim 131, wherein the molecule comprises a polypeptide chain comprising the amino acid sequence of SEQ ID NO:34.

134. A molecule, optionally the molecule of any one of embodiments 1 to 127, comprising a polypeptide chain comprising an amino acid sequence having at least 98% sequence identity to the amino acid sequence of SEQ ID NO:35.

135. The molecule of claim 134, wherein the molecule comprises a polypeptide chain comprising an amino acid sequence having at least 99% sequence identity to the amino acid sequence of SEQ ID NO:35.

136. The molecule of claim 134, wherein the molecule comprises a polypeptide chain comprising the amino acid sequence of SEQ ID NO:35.

137. A molecule, optionally the molecule of any one of embodiments 1 to 127, comprising a polypeptide chain comprising an amino acid sequence having at least 98% sequence identity to the amino acid sequence of SEQ ID NO:36.

138. The molecule of claim 137, wherein the molecule comprises a polypeptide chain comprising an amino acid sequence having at least 99% sequence identity to the amino acid sequence of SEQ ID NO:36.

139. The molecule of claim 137, wherein the molecule comprises a polypeptide chain comprising the amino acid sequence of SEQ ID NO:36.

140. A molecule, optionally the molecule of any one of embodiments 1 to 127, comprising a polypeptide chain comprising an amino acid sequence having at least 98% sequence identity to the amino acid sequence of SEQ ID NO:37.

141. The molecule of claim 140, wherein the molecule comprises a polypeptide chain comprising an amino acid sequence having at least 99% sequence identity to the amino acid sequence of SEQ ID NO:37.

142. The molecule of claim 140, wherein the molecule comprises a polypeptide chain comprising the amino acid sequence of SEQ ID NO:37.

143. A molecule, optionally the molecule of any one of embodiments 1 to 127, comprising a polypeptide chain comprising an amino acid sequence having at least 98% sequence identity to the amino acid sequence of SEQ ID NO:38.

144. The molecule of claim 143, wherein the molecule comprises a polypeptide chain comprising an amino acid sequence having at least 99% sequence identity to the amino acid sequence of SEQ ID NO:38.

145. The molecule of claim 143, wherein the molecule comprises a polypeptide chain comprising a polypeptide chain comprising the amino acid sequence of SEQ ID NO:38.

146. A molecule comprising a polypeptide chain comprising:
(a) a first amino acid sequence having at least 90% sequence identity to the amino acid sequence of SEQ ID NO:1; and
(b) a second amino acid sequence having at least 90% sequence identity to the amino acid sequence of SEQ ID NO: 6, SEQ ID NO:7, SEQ ID NO:8, or SEQ ID NO:9.

147. The molecule of embodiment 146, wherein the second amino acid sequence has at least 90% sequence identity to the amino acid sequence of SEQ ID NO: 6.

148. The molecule of embodiment 146, wherein the second amino acid sequence has at least 95% sequence identity to the amino acid sequence of SEQ ID NO: 6.

149. The molecule of embodiment 146, wherein the second amino acid sequence has at least 98% sequence identity to the amino acid sequence of SEQ ID NO: 6.

150. The molecule of embodiment 146, wherein the second amino acid sequence has at least 90% sequence identity to the amino acid sequence of SEQ ID NO: 7.

151. The molecule of embodiment 146, wherein the second amino acid sequence has at least 95% sequence identity to the amino acid sequence of SEQ ID NO: 7.

152. The molecule of embodiment 146, wherein the second amino acid sequence has at least 98% sequence identity to the amino acid sequence of SEQ ID NO: 7.

153. The molecule of embodiment 146, wherein the second amino acid sequence has at least 90% sequence identity to the amino acid sequence of SEQ ID NO: 8.

154. The molecule of embodiment 146, wherein the second amino acid sequence has at least 95% sequence identity to the amino acid sequence of SEQ ID NO: 8.

155. The molecule of embodiment 146, wherein the second amino acid sequence has at least 98% sequence identity to the amino acid sequence of SEQ ID NO: 8.

156. The molecule of embodiment 146, wherein the second amino acid sequence has at least 90% sequence identity to the amino acid sequence of SEQ ID NO: 9.

157. The molecule of embodiment 146, wherein the second amino acid sequence has at least 95% sequence identity to the amino acid sequence of SEQ ID NO: 9.

158. The molecule of embodiment 146, wherein the second amino acid sequence has at least 98% sequence identity to the amino acid sequence of SEQ ID NO: 9.

159. The molecule of any one of embodiments 146 to 158, wherein the second amino acid sequence has at least 98% sequence identity to the amino acid sequence of any one of SEQ ID NOS: 10-26.

160. The molecule of any one of embodiments 146 to 158, wherein the second amino acid sequence has at least 99% sequence identity to the amino acid sequence of any one of SEQ ID NOS: 10-26.

161. The molecule of any one of embodiments 146 to 158, wherein the second amino acid sequence comprises the amino acid sequence of any one of SEQ ID NOS: 10-26.

162. The molecule of any one of embodiments 146 to 161, wherein the first amino acid sequence has at least 95% sequence identity to the amino acid sequence of SEQ ID NO:1.

163. The molecule of any one of embodiments 146 to 161, wherein the first amino acid sequence has at least 98% sequence identity to the amino acid sequence of SEQ ID NO:1.

164. The molecule of any one of embodiments 146 to 161, wherein the first amino acid sequence comprises the amino acid sequence of SEQ ID NO:1.

165. The molecule of any one of embodiments 146 to 164, wherein the second amino acid sequence is C-terminal to the first amino acid sequence.

166. The molecule of any one of embodiments 146 to 164, wherein the second amino acid sequence is immediately C-terminal to the first amino acid sequence.

167. The molecule of any one of embodiments 146 to 166, which lacks an amino acid sequence having at least 90% identity to SEQ ID NO:2.

168. The molecule of any one of embodiments 146 to 167, which lacks the amino acid of SEQ ID NO:3.

169. The molecule of any one of embodiments 146 to 167, which lacks the amino acid of SEQ ID NO:4.

170. The molecule of any one of embodiments 146 to 167, which lacks the amino acid of SEQ ID NO:5.

171. A nucleic acid or plurality of nucleic acids encoding the molecule of any one of embodiments 1 to 170.

172. A host cell engineered to express the molecule of any one of embodiments 1 to 170.

173. A cell transfected with one or more expression vectors comprising one or more nucleic acid sequences encoding the molecule of any one of embodiments 1 to 170 under the control of one or more promoters.

174. A method of producing the molecule of any one of embodiments 1 to 170, comprising culturing the host cell of embodiment 172 or 173 and recovering the molecule expressed thereby.

175. A pharmaceutical composition comprising the molecule of any one of embodiments 1 to 170 and an excipient.

176. A method of agonizing or potentiating agonism of a target molecule comprising contacting the target molecule with a binding molecule, optionally a binding molecule of any one of embodiments 106 to 170, comprising:
(a) a first polypeptide chain comprising, from N- to C-terminal orientation:
   (i) a first component of a first targeting moiety;
   (ii) a first IgM Cμ2 domain;
   (iii) a first IgG CH2 domain; and
   (iv) a first IgG CH3 domain; and
(b) a second polypeptide chain comprising, from N- to C-terminal orientation:
   (i) a first component of a second targeting moiety;
   (ii) a second IgM Cμ2 domain;
   (iii) a second IgG CH2 domain; and
   (iv) a second IgG CH3 domain.

177. The method of embodiment 176, wherein the first component of the first targeting moiety comprises a VH domain.

178. The method of embodiment 176, wherein the first component of the first targeting moiety comprises a VL domain.

179. The method of any one of embodiments 176 to 178, wherein the first targeting moiety is a Fab.

180. The method of any one of embodiments 176 to 179, further comprising a third polypeptide chain comprising a second component of the first targeting moiety associated with the first component of the first targeting moiety.

181. The method of any one of embodiments 176 to 180, wherein the first component of the second targeting moiety comprises a VH domain.

182. The method of any one of embodiments 176 to 180, wherein the first component of the second targeting moiety comprises a VL domain.

183. The method of any one of embodiments 176 to 182, wherein the second targeting moiety is a Fab.

184. The method of any one of embodiments 176 to 183, further comprising a fourth polypeptide chain comprising a second component of the second targeting moiety associated with the first component of the second targeting moiety.

185. The method of any one of embodiments 176 to 184, further comprising contacting the target molecule with a ligand for the target molecule.

186. The method of any one of embodiments 176 to 185, wherein the contacting of the target molecule is performed in the presence of a ligand for the target molecule.

187. The method of any one of embodiments 176 to 186, wherein the method is performed in vivo.

188. The method of embodiment 187, wherein the method comprises administering the binding molecule to a subject in need thereof.

189. The method of any one of embodiments 176 to 186, wherein the method is performed in vitro or ex vivo.

190. The method of any one of embodiments 176 to 189, wherein the first and second targeting moieties each comprise an antigen binding domain of an agonist antibody.

191. The method of any one of embodiments 176 to 189, wherein the first and second targeting moieties each comprise an antigen binding domain of an antibody set forth in Table B.

192. The method of any one of embodiments 176 to 191, wherein the first and second targeting moieties each bind to a target molecule.

193. The method of embodiment 192, wherein the first and second targeting moieties each bind to different target molecules.

194. The method of embodiment 192, wherein the first and second targeting moieties each bind to the same target molecule.

195. The method of any one of embodiments 192 to 194, wherein the target molecule is a receptor.

196. The method of embodiment 195, wherein the target molecule is CD40, OX40, GITR, 4-1BB, CD27, HVEM, CD30, leptin receptor, or IGF1R.

197. The method of embodiment 195, wherein the target molecule is a TNF family receptor.

198. The method of embodiment 197, wherein the target molecule is CD40, OX40, GITR, 4-1BB, CD27, HVEM, or CD30.

199. The method of embodiment 195, wherein the target molecule is a homodimer class I cytokine receptor.

200. The method of embodiment 199, wherein the target molecule is EPO receptor, G-CSF receptor, leptin receptor (LEPR), or PRLR.

201. The method of embodiment 199 or 200, wherein the target molecule is leptin receptor (LEPR).

202. A method of treating a disease or condition associated with or caused by leptin deficiency or leptin resistance, the method comprising administering to a subject in need thereof a binding molecule, optionally the binding molecule of embodiment 127, comprising:
(a) a first polypeptide chain comprising, from N- to C-terminal orientation:
   (i) a first component of a first leptin receptor targeting moiety;
   (ii) a first IgM Cμ2 domain;
   (iii) a first IgG CH2 domain; and
   (iv) a first IgG CH3 domain; and
(b) a second polypeptide chain comprising, from N- to C-terminal orientation:
   (i) a first component of a second leptin receptor targeting moiety;
   (ii) a second IgM Cμ2 domain;
   (iii) a second IgG CH2 domain; and
   (iv) a second IgG CH3 domain.

203. The method of embodiment 202, wherein the disease or condition is obesity, metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome, or Rabson-Mendenhall syndrome.

204. The method of embodiment 202, wherein the disease or condition is generalized lipodystrophy, acquired generalized lipodystrophy, familial partial lipodystrophy, acquired partial lipodystrophy, centrifugal abdominal lipodystrophy, lipoatrophia annularis, localized lipodystrophy, or HIV-associated lipodystrophy.

205. The method of any one of embodiments 202 to 204, wherein the first component of the first leptin receptor targeting moiety comprises a VH domain.

206. The method of any one of embodiments 202 to 204, wherein the first component of the first leptin receptor targeting moiety comprises a VL domain.

207. The method of any one of embodiments 202 to 206, wherein the first leptin receptor targeting moiety is a Fab.

208. The method of any one of embodiments 202 to 207, further comprising a third polypeptide chain comprising a second component of the first leptin receptor targeting moiety associated with the first component of the first leptin receptor targeting moiety.

209. The method of any one of embodiments 202 to 208, wherein the first component of the second leptin receptor targeting moiety comprises a VH domain.

210. The method of any one of embodiments 202 to 208, wherein the first component of the second leptin receptor targeting moiety comprises a VL domain.

211. The method of any one of embodiments 202 to 210, wherein the second leptin receptor targeting moiety is a Fab.

212. The method of any one of embodiments 202 to 211, further comprising a fourth polypeptide chain comprising a second component of the second leptin receptor targeting moiety associated with the first component of the second leptin receptor targeting moiety.

213. A method of activating an immune response, the method comprising administering to a subject in need thereof a binding molecule, optionally the binding molecule of embodiment 123 or 124, comprising.
(a) a first polypeptide chain comprising, from N- to C-terminal orientation:
   (i) a first component of a first TNF family receptor targeting moiety;
   (ii) a first IgM Cμ2 domain;
   (iii) a first IgG CH2 domain; and
   (iv) a first IgG CH3 domain; and
(b) a second polypeptide chain comprising, from N- to C-terminal orientation:
   (i) a first component of a second TNF family receptor targeting moiety;
   (ii) a second IgM Cμ2 domain;
   (iii) a second IgG CH2 domain; and
   (iv) a second IgG CH3 domain.

214. The method of embodiment 213, wherein subject has cancer.

215. The method of embodiment 213, wherein the subject is at risk of developing cancer.

216. The method of any one of embodiments 213 to 215, wherein the first component of the first TNF family receptor targeting moiety comprises a VH domain.

217. The method of any one of embodiments 213 to 215, wherein the first component of the first TNF family receptor targeting moiety comprises a VL domain.

218. The method of any one of embodiments 213 to 217, wherein the first TNF family receptor targeting moiety is a Fab.

219. The method of any one of embodiments 213 to 218, further comprising a third polypeptide chain comprising a second component of the first TNF family receptor targeting moiety associated with the first component of the first TNF family receptor targeting moiety.

220. The method of any one of embodiments 213 to 219, wherein the first component of the second TNF family receptor targeting moiety comprises a VH domain.

221. The method of any one of embodiments 213 to 219, wherein the first component of the second TNF family receptor targeting moiety comprises a VL domain.

222. The method of any one of embodiments 213 to 221, wherein the second TNF family receptor targeting moiety is a Fab.

223. The method of any one of embodiments 213 to 222, further comprising a fourth polypeptide chain comprising a second component of the second TNF family receptor targeting moiety associated with the first component of the second TNF family receptor targeting moiety.

224. The method of any one of embodiments 213 to 223, wherein the first TNF family receptor targeting moiety is a CD40, OX40, GITR, 4-1BB, CD27, HVEM, or CD30 targeting moiety.

225. The method of embodiment 224, wherein the first TNF family receptor targeting moiety is a CD40 targeting moiety.

226. The method of embodiment 224, wherein the first TNF family receptor targeting moiety is a OX40 targeting moiety.

227. The method of embodiment 224, wherein the first TNF family receptor targeting moiety is a 4-1BB targeting moiety.

228. The method of any one of embodiments 213 to 227, wherein the second TNF family receptor targeting moiety is a CD40, OX40, GITR, 4-1BB, CD27, HVEM, or CD30 targeting moiety.

229. The method of embodiment 228, wherein the second TNF family receptor targeting moiety is a CD40 targeting moiety.

230. The method of embodiment 228, wherein the second TNF family receptor targeting moiety is a OX40 targeting moiety.

231. The method of embodiment 228, wherein the second TNF family receptor targeting moiety is a 4-1 BB targeting moiety.

### 8. EXAMPLES

### 8.1. Materials and Methods

### 8.1.1. Design and Production of Cμ2-IgG Anti-LEPR Constructs

Exemplary Cμ2-IgG anti-LEPR antibodies, configured as depicted in FIG. 1D, were designed. The constructs were expressed in Expi293F™ cells by transient transfection following the manufacturer's protocol (Thermo Fisher Scientific). Proteins in Expi293F™ supernatant were purified using the ProteinMaker system (Protein BioSolutions, Gaithersburg, MD) with HiTrap™ MabSelect SuRe columns (Cytiva). After single step elution, the constructs were neutralized, dialyzed into a final buffer of phosphate buffered saline (PBS) with 5% glycerol, aliquoted and stored at −80° C. until use.

Additional details of the evaluated anti-LEPR antibody constructs are provided in Table E1 below.

TABLE E1

| Construct | Description |
|---|---|
| LEPR(Ab1)-Cμ2-IgG4(1) | Anti-LEPR (Ab1) - IgM Cμ2 - IgG4 Fc |
| LEPR(Ab1)-Cμ2-IgG2(1) | Anti-LEPR (Ab1) - IgM Cμ2 - IgG2 Fc |
| LEPR(Ab1)-Cμ2-IgG4(2) | Anti-LEPR (Ab1) - IgM (+PLP) Cμ2 - IgG4 Fc |
| LEPR(Ab1)-Cμ2-IgG2(2) | Anti-LEPR (Ab1) - IgM (+PLP) Cμ2 - IgG2 Fc |
| LEPR(Ab1)-Cμ2-IgG4(3) | Anti-LEPR (Ab1) - IgM (+PLP) Cμ2 - |
| | (−AP) IgG4 Fc |
| LEPR(Ab1)-Cμ2-IgG2(3) | Anti-LEPR (Ab1) - IgM (+PLP) Cμ2 - |
| | (−AP) IgG4 Fc |
| LEPR(Ab3)-Cμ2-IgG4(1) | Anti-LEPR (Ab3) - IgM Cμ2 - IgG4 Fc |
| LEPR(Ab3)-Cμ2-IgG2(1) | Anti-LEPR (Ab3) - IgM Cμ2 - IgG2 Fc |
| LEPR(Ab3)-Cμ2-IgG4(2) | Anti-LEPR (Ab3) - IgM (+PLP) Cμ2 - IgG4 Fc |

In each of the constructs of Table E1, the CH1, CH2, and CH3 sequences are those of the indicated IgG subclass. For example, the construct designated as LEPR(Ab1)-Cμ2-IgG4(1) has CH1, CH2, and CH3 sequences of IgG4. The hinge region of each IgG construct was replaced with an IgM Cμ2 region.

Sequences of the IgM Cμ2-IgG Fc portion of each of the above constructs are provided in Table E2 below, with Cμ2 portions underlined.

TABLE E2

| | | SEQ ID |
|---|---|---|
| Structure | Sequence | NO: |
| IgM Cμ2 - | VIAELPPKVSVFVPPRDGFFGNPRKSKLICQATGFSPRQIQVSW | 33 |
| IgG4 Fc | LREGKQVGSGVTTDQVQAEAKESGPTTYKVTSTLTIKESDWLGQ | |
| | SMFTCRVDHRGLTFQQNASSMCVPAPEFLGGPSVFLFPPKPKDT | |
| | LMISRTPEVTCVVVDVSQEDPEVQFNWYVDGVEVHNAKTKPREE | |
| | QFNSTYRVVSVLTVLHQDWLNGKEYKCKVSNKGLPSSIEKTISK | |
| | AKGQPREPQVYTLPPSQEEMTKNQVSLTCLVKGFYPSDIAVEWE | |
| | SNGQPENNYKTTPPVLDSDGSFFLYSRLTVDKSRWQEGNVFSCS | |
| | VMHEALHNHYTQKSLSLSLGK | |
| IgM Cμ2 - | VIAELPPKVSVFVPPRDGFFGNPRKSKLICQATGFSPRQIQVSW | 34 |
| IgG2 Fc | LREGKQVGSGVTTDQVQAEAKESGPTTYKVTSTLTIKESDWLGQ | |
| | SMFTCRVDHRGLTFQQNASSMCVPAPPVAGPSVFLFPPKPKDTL | |
| | MISRTPEVTCVVVDVSHEDPEVQFNWYVDGVEVHNAKTKPREEQ | |
| | FNSTFRVVSVLTVVHQDWLNGKEYKCKVSNKGLPAPIEKTISKT | |
| | KGQPREPQVYTLPPSREEMTKNQVSLTCLVKGFYPSDIAVEWES | |
| | NGQPENNYKTTPPMLDSDGSFFLYSKLTVDKSRWQQGNVFSCSV | |
| | MHEALHNHYTQKSLSLSPGK | |
| IgM (+PLP) | PLPVIAELPPKVSVFVPPRDGFFGNPRKSKLICQATGFSPRQIQ | 35 |
| Cμ2 - IgG4 | VSWLREGKQVGSGVTTDQVQAEAKESGPTTYKVTSTLTIKESDW | |
| Fc | LGQSMFTCRVDHRGLTFQQNASSMCVPAPEFLGGPSVFLFPPKP | |
| | KDTLMISRTPEVTCVVVDVSQEDPEVQFNWYVDGVEVHNAKTKP | |
| | REEQFNSTYRVVSVLTVLHQDWLNGKEYKCKVSNKGLPSSIEKT | |
| | ISKAKGQPREPQVYTLPPSQEEMTKNQVSLTCLVKGFYPSDIAV | |
| | EWESNGQPENNYKTTPPVLDSDGSFFLYSRLTVDKSRWQEGNVF | |
| | SCSVMHEALHNHYTQKSLSLSLGK | |
| IgM (+PLP) | PLPVIAELPPKVSVFVPPRDGFFGNPRKSKLICQATGFSPRQIQ | 36 |
| Cμ2 - IgG2 | VSWLREGKQVGSGVTTDQVQAEAKESGPTTYKVTSTLTIKESDW | |
| Fc | LGQSMFTCRVDHRGLTFQQNASSMCVPAPPVAGPSVFLFPPKPK | |
| | DTLMISRTPEVTCVVVDVSHEDPEVQFNWYVDGVEVHNAKTKPR | |
| | EEQFNSTFRVVSVLTVVHQDWLNGKEYKCKVSNKGLPAPIEKTI | |
| | SKTKGQPREPQVYTLPPSREEMTKNQVSLTCLVKGFYPSDIAVE | |
| | WESNGQPENNYKTTPPMLDSDGSFFLYSKLTVDKSRWQQGNVFS | |
| | CSVMHEALHNHYTQKSLSLSPGK | |
| IgM (+PLP) | PLPVIAELPPKVSVFVPPRDGFFGNPRKSKLICQATGFSPRQIQ | 37 |
| Cμ2 - (−AP) | VSWLREGKQVGSGVTTDQVQAEAKESGPTTYKVTSTLTIKESDW | |
| IgG4 Fc | LGQSMFTCRVDHRGLTFQQNASSMCVPEFLGGPSVFLFPPKPKD | |
| | TLMISRTPEVTCVVVDVSQEDPEVQFNWYVDGVEVHNAKTKPRE | |
| | EQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEW | |
| | ESNGQPENNYKTTPPVLDSDGSFFLYSRLTVDKSRWQEGNVFSC | |
| | SVMHEALHNHYTQKSLSLSLGK | |
| IgM (+PLP) | PLPVIAELPPKVSVFVPPRDGFFGNPRKSKLICQATGFSPRQIQ | 38 |
| Cμ2 - (−AP) | VSWLREGKQVGSGVTTDQVQAEAKESGPTTYKVTSTLTIKESDW | |
| IgG4 Fc | LGQSMFTCRVDHRGLTFQQNASSMCVPPVAGPSVFLFPPKPKDT | |
| | LMISRTPEVTCVVVDVSHEDPEVQFNWYVDGVEVHNAKTKPREE | |
| | QFNSTFRVVSVLTVVHQDWLNGKEYKCKVSNKGLPAPIEKTISK | |
| | TKGQPREPQVYTLPPSREEMTKNQVSLTCLVKGFYPSDIAVEWE | |
| | SNGQPENNYKTTPPMLDSDGSFFLYSKLTVDKSRWQQGNVFSCS | |
| | VMHEALHNHYTQKSLSLSPGK | |

### 8.1.2. Negative Stain EM

Negative stain electron microscopy (EM) was conducted to qualitatively assess the molecular structures of Cμ2-IgG anti-LEPR constructs.

Purified samples at a protein concentration of approximately 0.02 mg/mL were applied to 400 mesh carbon film Cu grids (Electron Microscopy Sciences) and negative-stained with NanoW (Nanoprobes) or Vitroease Methylamine Tungstate (Thermo Fisher).

Negative stain EM grids were inserted into a Glacios TEM (Thermo Fisher) and imaged with a Ceta camera (Thermo Fisher). Automated data collection was performed at a nominal magnification of 73,000x and 2A pixel size using EPU. A total of 550 micrographs were collected, and EM data were processed using RELION 4.0, wherein 450,000 particles were picked using the Laplacian of Gaussian (LoG) algorithm to generate 2D templates that were subsequently used for template-based particle picking. Particle images were subjected to multiple rounds of 2D classification, selecting particles belonging to class averages with clear features after each round.

### 8.1.3. STAT3 Reporter Assay

A Signal Transducer and Activator of Transcription 3 (STAT3)-driven luciferase-based reporter assay was used to evaluate the ability of Cμ2-IgG anti-LEPR constructs to activate STAT3-mediated transcription in IMR32/STAT3-Luc/hLEPR clone B10 cells. Briefly, cells were transduced with a STAT3 response element driven luciferase reporter construct and maintained in alpha minimum essential medium without nucleosides+2 mM L-glutamine/Pen/Strep+1.5 g/L sodium bicarbonate+12.5% horse serum+12.5% FBS+0.2 mM inositol+0.1 mM 2-mercaptoethanol+0.02 mM folic acid+200U/mL recombinant hlL2+1 mg/mL puromycin.

RPM11640 supplemented with 10% FBS and P/S/G was used as assay medium to prepare cell suspensions and construct dilutions. A day prior to the assay, cells were spun down and resuspended at 5×10⁵ cells/mL in alpha minimum essential medium without nucleosides+2 mM L-glutamine/Pen/Strep+1.5 g/L sodium bicarbonate+12.5% horse serum+12.5% FBS+0.2 mM inositol+0.1 mM 2-mercaptoethanol+0.02 mM folic acid. On the day of the assay, in IMR32/STAT3-Luc/hLEPR clone B10 cells were spun down, resuspended in assay medium and added to plates at 2.5×10⁴ cells/well. Constructs were serially diluted (range: 50 nM to 29.8fM) and added to cells for 4 hours at 37° C. and 5% CO₂ prior to addition of One-Glo Luciferase Substrate to lyse cells and detect luciferase activity. The emitted light was captured in relative light units (RLU) on a multilabel plate reader Envision (PerkinElmer). All serial dilutions were tested in duplicates. EC50 values of the antibodies were determined using GraphPad Prism™ software from a four-parameter logistic equation over a 10-point dose-response curve.

### 8.2. Example 1: Structural Assessment of Cμ2-IgG Anti-LEPR Antibodies

To determine whether the angle between the Fab arms of Cμ2-IgG antibody constructs would differ from the angle between the Fab arms of unmodified IgG antibodies, anti-LEPR(Ab1)-Cμ2-IgG2, anti-LEPR(Ab1)-Cμ2-IgG4, and their unmodified IgG2 and IgG4 controls were assessed with negative stain EM as described in Section 8.1.2.

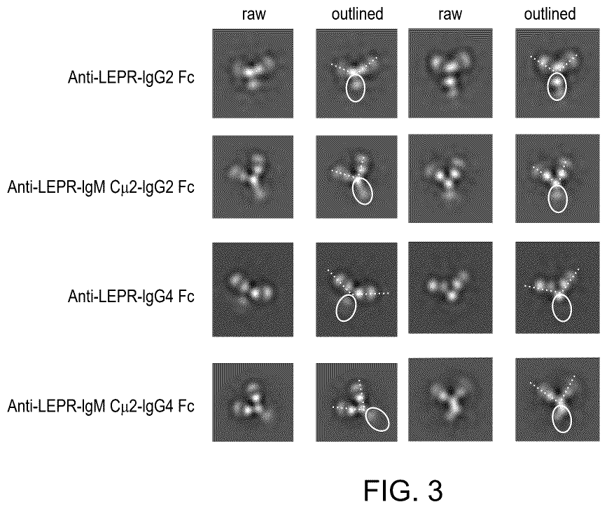

Anti-LEPR(Ab1)-Cμ2-IgG2 and anti-LEPR(Ab1)-Cμ2-IgG4 were associated with acute angles between their Fab arms, which was narrower than the angles between the Fab arms of unmodified anti-LEPR(Ab1)-IgG2 and anti-LEPR(Ab1)-IgG4 antibodies among the representative images (FIG. 3). Thus, replacement of the hinge region of IgG Fc with IgM Cμ2 resulted in Fab arms having a more “closed” and rigid conformation relative to wild type IgG antibodies lacking the IgM Cμ2.

### 8.3. Example 2: Activation of Lepr-Dependent Stat3-Signaling by Cμ2-Igg2-Containing Anti-Lepr Constructs

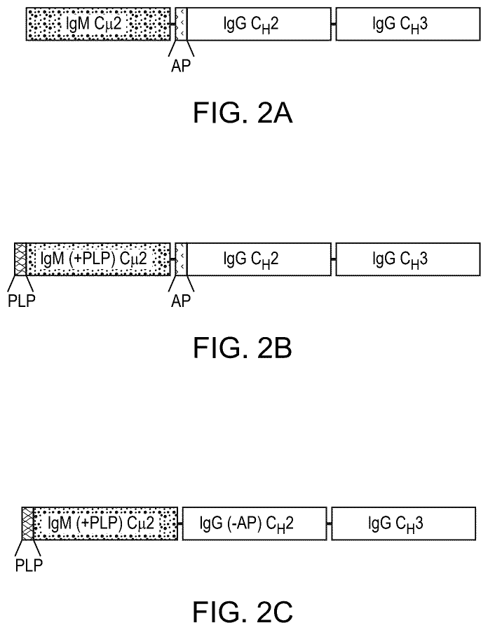

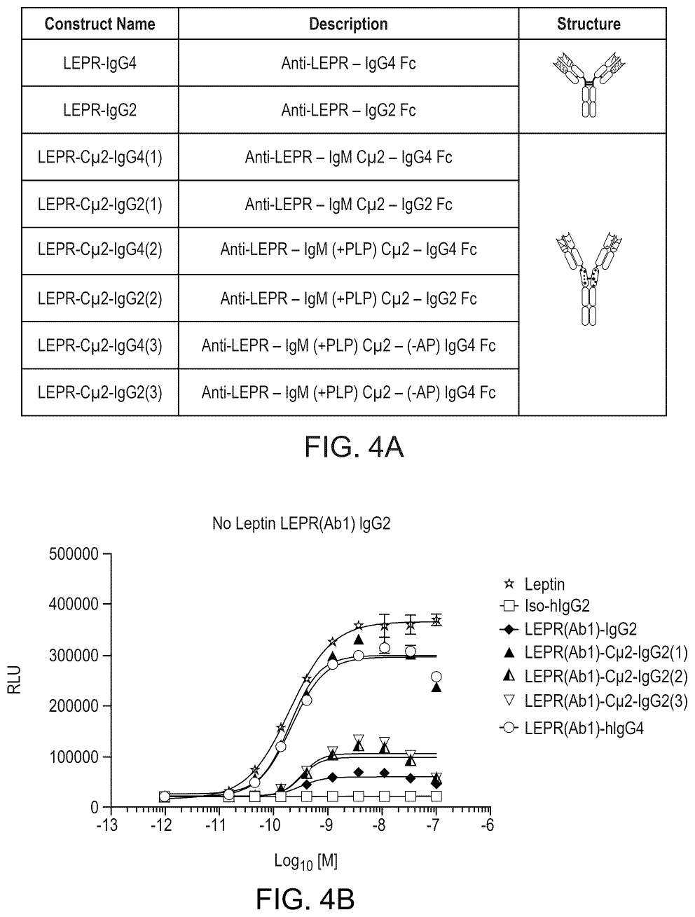

Three anti-LEPR(Ab1)-Cμ2-IgG2 constructs were designed and generated as described in Section 8.1.1. The construct anti-LEPR(Ab1)-Cμ2-IgG2(1) retained the amino acids “AP” at the N-terminus of the IgG2 CH2 domain (as depicted in FIG. 2A). The construct anti-LEPR(Ab1)-Cμ2-IgG2(2) was modified by adding the amino acids PLP to the N-terminus of the IgM Cμ2 domain (as depicted in FIG. 2B). The construct anti-LEPR(Ab1)-Cμ2-IgG2(3) was modified by adding the amino acids “PLP” to the N-terminus of the IgM Cμ2 domain and by removing the amino acids “AP” at the N-terminus of the IgG2 CH2 domain (as depicted in FIG. 2C). FIG. 4A provides an overview of the constructs generated.

Next, the ability of the three anti-LEPR(Ab1)-Cμ2-IgG2 constructs and controls to activate LEPR-dependent STAT3 signaling in the absence of leptin was assessed using a STAT3-reporter cell-based assay as described in Section 8.1.3.

The control anti-LEPR(1) antibody with an IgG4 backbone was associated with a robust activation of the LEPR-dependent STAT3-signaling, whereas anti-LEPR(1) antibody with an IgG2 backbone displayed no activation. However, replacing the IgG2 CH2 hinge region with IgM-Cμ2 enhanced the STAT3-activation activity of the anti-LEPR(Ab1)-Cμ2-IgG2(1) construct to similar level as the control anti-LEPR(1) antibody with the IgG4 backbone (FIG. 4B).

### 8.4. Example 3: Activation of Lepr-Dependent Stat3-Signaling by Cμ2-Igg4-Containing Anti-Lepr Constructs

Anti-LEPR antibodies Ab1 and Ab3 differ in their ability to activate LEPR-dependent STAT3 signaling. To determine whether replacing the IgG4 CH2 hinge region with IgM-Cμ2 could enhance the activity of anti-LEPR(Ab3) antibody, the construct anti-LEPR(Ab3)-Cμ2-IgG4(1) was generated as described in Section 8.1.1 and its ability to activate LEPR-dependent STAT3 signaling against controls in the absence and presence of leptin was assessed using a STAT3-reporter cell-based assay as described in Section 8.1.3.

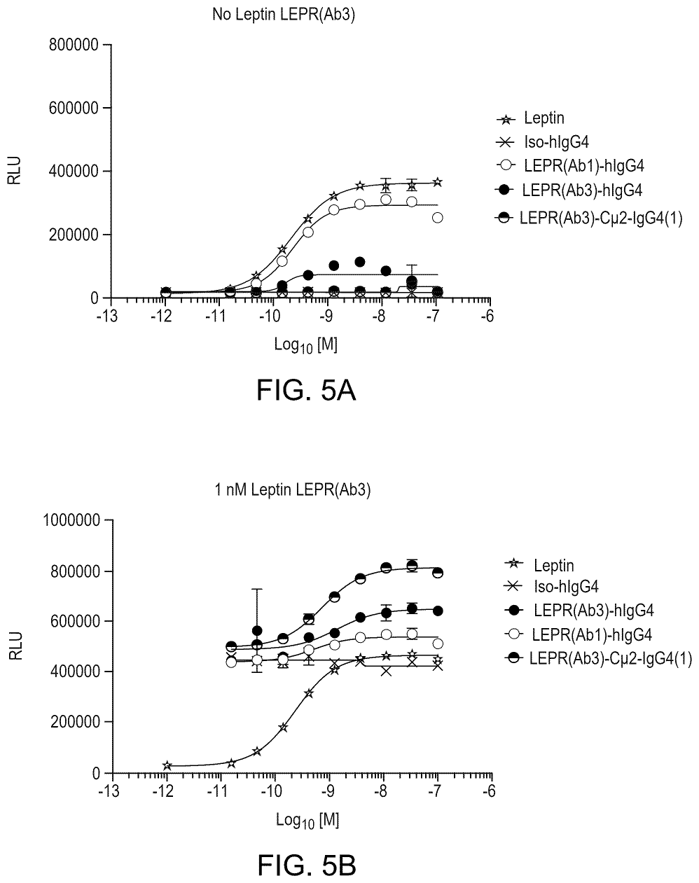

In the absence of leptin, the control antibody anti-LEPR(Ab1)-hIgG4 was able to activate the LEPR-dependent STAT3 signaling, anti-LEPR(Ab3)-hIgG4 displayed a minimum level of activation of LEPR-dependent STAT3 signaling, and the anti-LEPR(Ab3)-Cμ2-IgG4(1) construct did not display any activation of LEPR-dependent STAT3 signaling (FIG. 5A). In the presence of 1 nM leptin, anti-LEPR(Ab3)-Cμ2-IgG4(1) was associated with a significant potentiation of LEPR-dependent STAT3 signaling that was higher than potentiation obtained with the control anti-LEPR(Ab1) or anti-LEPR(Ab3) antibodies (FIG. 5B), highlighting that replacing the IgG4 hinge region with IgM-Cμ2 domain was able to enhance the activity of anti-LEPR(Ab3) antibody.

### 8.5. Example 4: Effect of IgM-Cμ2 Replacement on an Antagonistic Anti-LEPR Antibody

Anti-LEPR antibody Ab6, in its wildtype IgG format, antagonizes leptin-induced activation of LEPR-dependent STAT3 signaling. To determine whether replacing the IgG4 CH2 hinge region of Ab6 with IgM-Cμ2 could alter its function, the construct anti-LEPR(Ab6)-Cμ2-IgG4(1) was generated as described in Section 8.1.1 and its ability to activate LEPR-dependent STAT3 signaling against controls in the presence of leptin was assessed using a STAT3-reporter cell-based assay as described in Section 8.1.3.

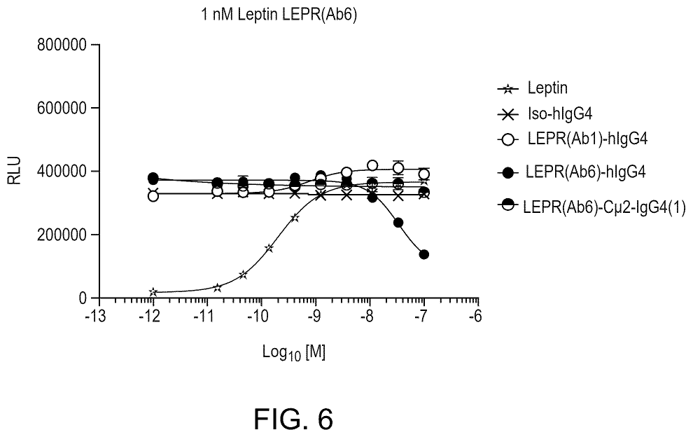

In the presence of 1 nM leptin, anti-LEPR(Ab6)-IgG4 displayed a dose-dependent inhibition of leptin-induced activation of LEPR-dependent STAT3 signaling (FIG. 6). In contrast, anti-LEPR(Ab6)-Cμ2-IgG4(1) neither inhibited nor potentiated LEPR-dependent STAT3 signaling at any concentration evaluated (FIG. 6), suggesting that replacement of the IgG4 hinge region with IgM-Cμ2 converted antagonistic anti-LEPR antibody Ab6 into a non-active antibody (i.e., one that has minimal impact on LEPR signaling).

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A molecule comprising a first polypeptide chain comprising, in N- to C-terminal orientation:
(a) an IgG constant domain;
(b) an IgM Cμ2 domain;
(c) an IgG CH2 domain; and
(d) an IgG CH3 domain.

2. The molecule of claim 1, wherein the first polypeptide chain lacks an intact IgG hinge domain.

3. The molecule of claim 1 or 2, wherein the first polypeptide chain has an IgG hinge domain of reduced length relative to a wild type IgG hinge domain.

4. The molecule of claim 2 or 3, wherein the IgG hinge domain is an IgG2, an IgG4, or an IgG1 hinge domain.

5. The molecule of any one of claims 1 to 4, which lacks an IgM Cμ1 domain.

6. The molecule of claim 5, wherein the first polypeptide chain lacks an amino acid sequence having at least 90% sequence identity to the amino acid sequence of SEQ ID NO:2.

7. The molecule of any one of claims 1 to 6, wherein the IgG constant domain is an IgG CH1 domain.

8. The molecule of claim 7, wherein the IgG CH1 domain is an IgG2 CH1 domain, an IgG4 CH1 domain, or an IgG1 CH1 domain.

9. The molecule of claim 7 or 8, wherein the first polypeptide chain further comprises, N-terminal to the IgG CH1 domain, a VH domain.

10. The molecule of any one of claims 1 to 6, wherein the IgG constant domain is an IgG CL domain.

11. The molecule of claim 10, wherein the first polypeptide chain further comprises, N-terminal to the IgG CL domain, a VL domain.

12. The molecule of any one of claims 1 to 11, wherein the IgG CH2 domain is an IgG2 CH2 domain, an IgG4 CH2 domain, or an IgG1 CH2 domain.

13. The molecule of any one of claims 1 to 12, wherein the IgG CH3 domain is an IgG2 CH3 domain, an IgG4 CH3 domain, or an IgG1 CH3 domain.

14. The molecule of any one of claims 1 to 13, wherein the IgG CH2 domain comprises one or more amino acid substitutions that reduce binding to an Fc receptor.

15. The molecule of claim 14, wherein the Fc receptor is an Fcγ receptor (e.g., an FcγRIIIa receptor).

16. The molecule of any one of claims 1 to 13, wherein the IgG CH2 domain comprises one or more amino acid substitutions that increase binding to an Fc receptor (e.g., FcγRIIB).

17. The molecule of any one of claims 1 to 13, wherein the IgG CH3 domain comprises one or more amino acid substitutions that reduce binding to an Fc receptor.

18. The molecule of claim 17, wherein the Fc receptor is an Fcγ receptor (e.g., FcγRIIa).

19. The molecule of any one of claims 1 to 18, wherein the IgG CH3 domain comprises one or more amino acid substitutions that increase binding to an Fc receptor (e.g., FcγRIIB).

20. The molecule of any one of claims 1 to 19, wherein the IgG CH2 domain and IgG CH3 domain together comprise an amino acid sequence having at least 95%, at least 98%, or at least 99% sequence identity to an amino acid sequence set forth in Table F-1.

21. The molecule of any one of claims 1 to 19, wherein the IgG CH2 domain and IgG CH3 domain together comprise an amino acid sequence set forth in Table F-1.

22. The molecule of any one of claims 1 to 21, wherein the first polypeptide chain comprises an amino acid sequence having at least 95%, at least 98%, at least 99%, or at least 99.5% sequence identity to the amino acid sequence of SEQ ID NO:34.

23. The molecule of any one of claims 1 to 21, wherein the first polypeptide chain comprises the amino acid sequence of SEQ ID NO:34.

24. The molecule of any one of claims 1 to 21, wherein the first polypeptide chain comprises an amino acid sequence having at least 95%, at least 98%, at least 99%, or at least 99.5% sequence identity to the amino acid sequence of SEQ ID NO:33.

25. The molecule of any one of claims 1 to 21, wherein the first polypeptide chain comprises the amino acid sequence of SEQ ID NO:33.

26. The molecule of any one of claims 1 to 25, which comprises an Fc dimer.

27. The molecule of claim 26, wherein the Fc dimer is an Fc homodimer.

28. The molecule of any one of claims 1 to 27, wherein the molecule is a binding molecule.

29. The molecule of claim 28, which comprises a first targeting moiety.

30. The molecule of claim 29, wherein the first targeting moiety is a Fab.

31. The molecule of any one of claims 28 to 30, which comprises:
(a) a first polypeptide chain comprising, from N- to C-terminal orientation:
(i) a first targeting moiety or portion thereof;
(ii) a first IgM Cμ2 domain;
(iii) a first IgG CH2 domain; and
(iv) a first IgG CH3 domain;
(b) a second polypeptide chain comprising, from N- to C-terminal orientation:
(i) a second targeting moiety or portion thereof;
(ii) a second IgM Cμ2 domain;
(iii) a second IgG CH2 domain; and
(iv) a second IgG CH3 domain.

32. The molecule of claim 31, wherein
(a) the first polypeptide chain comprises, N-terminal to the first IgM Cμ2 domain, a first VH domain and a first IgG CH1 domain;
(b) the molecule further comprises a third polypeptide chain comprising a first VL domain and a first IgG CL domain, the third polypeptide chain associated with the first polypeptide chain to form the first targeting moiety;
(c) the second polypeptide chain comprises, N-terminal to the first IgM Cμ2 domain, a first VH domain and a first IgG CH1 domain; and
(d) the molecule further comprises a fourth polypeptide chain comprising a second VL domain and a second IgG CL domain, the fourth polypeptide chain associated with the second polypeptide chain to form the second targeting moiety.

33. The molecule of any one of claims 29 to 32, wherein the first targeting moiety and/or second targeting moiety comprises an antigen binding domain of an agonist antibody.

34. The molecule of any one of claims 29 to 33, wherein the first targeting moiety and/or second targeting moiety binds to a target molecule.

35. The molecule of claim 34, wherein the target molecule is a receptor (e.g., CD40, OX40, GITR, 4-1 BB, CD27, HVEM, CD30, leptin receptor (LEPR), or IGF1R).

36. The molecule of claim 35, wherein the target molecule is a TNF family receptor (e.g., CD40, OX40, GITR, 4-1BB, CD27, HVEM, or CD30).

37. The molecule of claim 35, wherein the target molecule is a homodimer class I cytokine receptor (e.g., an EPO receptor, G-CSF receptor, leptin receptor (LEPR), or PRLR).

38. The molecule of claim 37, wherein the target molecule is leptin receptor (LEPR).

39. A nucleic acid or plurality of nucleic acids encoding the molecule of any one of claims 1 to 38.

40. A host cell engineered to express the molecule of any one of claims 1 to 38.

41. A cell transfected with one or more expression vectors comprising one or more nucleic acid sequences encoding the molecule of any one of claims 1 to 38 under the control of one or more promoters.

42. A method of producing the molecule of any one of claims 1 to 38, comprising culturing the host cell of claim 40 or 41 and recovering the molecule expressed thereby.

43. A pharmaceutical composition comprising the molecule of any one of claims 1 to 38 and an excipient.

44. A method of agonizing or potentiating agonism of a target molecule comprising contacting the target molecule with a binding molecule, optionally a binding molecule of any one of claims 28 to 38, comprising:
(a) a first polypeptide chain comprising, from N- to C-terminal orientation:
(i) a first component of a first targeting moiety;
(ii) a first IgM Cμ2 domain;
(iii) a first IgG CH2 domain; and
(iv) a first IgG CH3 domain; and
(b) a second polypeptide chain comprising, from N- to C-terminal orientation:
(i) a first component of a second targeting moiety;
(ii) a second IgM Cμ2 domain;
(iii) a second IgG CH2 domain; and
(iv) a second IgG CH3 domain.

45. The method of claim 44, wherein the first component of the first targeting moiety comprises a VH domain or a VL domain.

46. The method of claim 44 or 45, wherein the first targeting moiety is a Fab.

47. The method of any one of claims 44 to 46, wherein the first component of the second targeting moiety comprises a VH domain or a VL domain.

48. The method of any one of claims 44 to 47, wherein the second targeting moiety is a Fab.

49. The method of any one of claims 44 to 48, further comprising contacting the target molecule with a ligand for the target molecule.

50. The method of any one of claims 44 to 49, wherein the contacting of the target molecule is performed in the presence of a ligand for the target molecule.

51. The method of any one of claims 44 to 50, wherein the method comprises administering the binding molecule to a subject in need thereof.

52. The method of any one of claims 44 to 50, wherein the method is performed in vitro or ex vivo.

53. The method of any one of claims 44 to 50, wherein the first and second targeting moieties each comprise an antigen binding domain of an agonist antibody.

54. The method of any one of claims 44 to 53, wherein the first and second targeting moieties each bind to a target molecule.

55. The method of claim 54, wherein the first and second targeting moieties each bind to different target molecules.

56. The method of claim 54, wherein the first and second targeting moieties each bind to the same target molecule.

57. The method of any one of claims 54 to 56, wherein the target molecule is leptin receptor (LEPR).

58. A method of treating a disease or condition associated with or caused by leptin deficiency or leptin resistance, the method comprising administering to a subject in need thereof a binding molecule, optionally the binding molecule of claim 38, comprising:
(a) a first polypeptide chain comprising, from N- to C-terminal orientation:
(i) a first component of a first leptin receptor targeting moiety;
(ii) a first IgM Cμ2 domain;
(iii) a first IgG CH2 domain; and
(iv) a first IgG CH3 domain; and
(b) a second polypeptide chain comprising, from N- to C-terminal orientation:
(i) a first component of a second leptin receptor targeting moiety;
(ii) a second IgM Cμ2 domain;
(iii) a second IgG CH2 domain; and
(iv) a second IgG CH3 domain.

59. The method of claim 58, wherein the disease or condition is obesity, metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome, or Rabson-Mendenhall syndrome.

60. The method of claim 58, wherein the disease or condition is generalized lipodystrophy, acquired generalized lipodystrophy, familial partial lipodystrophy, acquired partial lipodystrophy, centrifugal abdominal lipodystrophy, lipoatrophia annularis, localized lipodystrophy, or HIV-associated lipodystrophy.

61. The method of any one of claims 58 to 60, wherein the first component of the first leptin receptor targeting moiety comprises a VH domain or a VL domain.

62. The method of any one of claims 58 to 61, wherein the first leptin receptor targeting moiety is a Fab.

63. The method of any one of claims 58 to 62, wherein the first component of the second leptin receptor targeting moiety comprises a VH domain or a VL domain.

64. The method of any one of claims 58 to 63, wherein the second leptin receptor targeting moiety is a Fab.

65. A method of activating an immune response, the method comprising administering to a subject in need thereof a binding molecule, optionally the binding molecule of claim 36, comprising.
(a) a first polypeptide chain comprising, from N- to C-terminal orientation:
(i) a first component of a first TNF family receptor targeting moiety;
(ii) a first IgM Cμ2 domain;
(iii) a first IgG CH2 domain; and
(iv) a first IgG CH3 domain; and
(b) a second polypeptide chain comprising, from N- to C-terminal orientation:
(i) a first component of a second TNF family receptor targeting moiety;
(ii) a second IgM Cμ2 domain;
(iii) a second IgG CH2 domain; and
(iv) a second IgG CH3 domain.

66. The method of claim 65, wherein subject has cancer or is at risk of developing cancer.

67. The method of claim 65 or 66, wherein the first component of the first TNF family receptor targeting moiety comprises a VH domain or a VL domain.

68. The method of any one of claims 65 to 67, wherein the first TNF family receptor targeting moiety is a Fab.

69. The method of any one of claims 65 to 68, wherein the first component of the second TNF family receptor targeting moiety comprises a VH domain or a VL domain.

70. The method of any one of claims 65 to 69, wherein the second TNF family receptor targeting moiety is a Fab.

71. The method of any one of claims 65 to 70, wherein the first TNF family receptor targeting moiety is a CD40, OX40, GITR, 4-1BB, CD27, HVEM, or CD30 targeting moiety.

72. The method of any one of claims 65 to 71, wherein the second TNF family receptor targeting moiety is a CD40, OX40, GITR, 4-1BB, CD27, HVEM, or CD30 targeting moiety.
